SEQUENCE LISTING (17 sequences)

SEQ ID NO: 1 - DNA, 20 bp, synthetic construct
  source 1..20: mol_type = other DNA
actcggtgccactttttcaa

SEQ ID NO: 2 - DNA, 68 bp, synthetic construct
  source 1..68: mol_type = other DNA
gtttcagagctatgctctcctgttcgccaaattctacccaccacccactctccaaaggacgaaacacc

SEQ ID NO: 3 - DNA, 43 bp, synthetic construct
  source 1..43: mol_type = other DNA
gccaaattctacccaccacccactctccaaaggacgaaacacc

SEQ ID NO: 4 - DNA, 196 bp, synthetic construct
  source 1..196: mol_type = other DNA
acattatcatgaacactacggccgacgtgagaattgaataataatagacgaaatcctaggcgcgttcgctgtacaccatacgatgtaataataatattgtaatcacgtcatggtaccggttgagattcacgtagataggatcatgcatcgccacacattacactgattaagaggcggaagcgtctatctggagtat

SEQ ID NO: 5 - DNA, 196 bp, synthetic construct
  source 1..196: mol_type = other DNA
atactccagatagacgcttccgcctcttaatcagtgtaatgtgtggcgatgcatgatcctatctacgtgaatctcaaccggtaccatgacgtgattacaatattattattacatcgtatggtgtacagcgaacgcgcctaggatttcgtctattattattcaattctcacgtcggccgtagtgttcatgataatgt

SEQ ID NO: 6 - DNA, 106 bp, synthetic construct
  source 1..106: mol_type = other DNA
acattacacgcattagaagtagcgttgactgtccccaataataatagacgaaatcctaggcgcgttatcgactacttgactacgccaataataatattgtaatcac

SEQ ID NO: 7 - DNA, 100 bp, synthetic construct
  source 1..100: mol_type = other DNA
atactctcccgacgccacaaacttaacgccgcctaataatgtgttcggcattgcaacttgtacgacgtatctagcacggaatgttcggacgtgattacaa

SEQ ID NO: 8 - DNA, 20 bp, synthetic construct
  source 1..20: mol_type = other DNA
gacgaaatcctaggcgcgtt

SEQ ID NO: 9 - DNA, 20 bp, synthetic construct
  source 1..20: mol_type = other DNA
atcgactacttgactacgcc

SEQ ID NO: 10 - DNA, 20 bp, synthetic construct
  source 1..20: mol_type = other DNA
cgtacaagttgcaatgccga

SEQ ID NO: 11 - DNA, 20 bp, synthetic construct
  source 1..20: mol_type = other DNA
ccgaacattccgtgctagat

SEQ ID NO: 12 - DNA, 24 bp, synthetic construct
  source 1..24: mol_type = other DNA
gtcgtacaagttgcaatgccgaac

SEQ ID NO: 13 - DNA, 24 bp, synthetic construct
  source 1..24: mol_type = other DNA
gtccgaacattccgtgctagatac

SEQ ID NO: 14 - DNA, 30 bp, synthetic construct
  source 1..30: mol_type = other DNA
cacgcattagaagtagcgttgactgtcccc

SEQ ID NO: 15 - DNA, 30 bp, synthetic construct
  source 1..30: mol_type = other DNA
tcccgacgccacaaacttaacgccgcctaa

SEQ ID NO: 16 - DNA, 30 bp, synthetic construct
  source 1..30: mol_type = other DNA
ggggacagtcaacgctacttctaatgcgtg

SEQ ID NO: 17 - DNA, 30 bp, synthetic construct
  source 1..30: mol_type = other DNA
ttaggcggcgttaagtttgtggcgtcggga

(19) United States
(12) Patent Application Publication
(10) Pub. No.: US 2026/0146279 A1
(43) Pub. Date: May 28, 2026

(54) METHODS FOR OPTICAL IN SITU NUCLEOTIDE SEQUENCE DETECTION

(71) Applicant: The Trustees of Columbia University in the City of New York, New York, NY (US)

(72) Inventor: Jellert Gaublomme, New York, NY (US)

(21) Appl. No.: 19/401,747

(22) Filed: Nov. 26, 2025

Related U.S. Application Data
(60) Provisional application No. 63/725,319, filed on Nov. 26, 2024.

(51) Int. Cl. C12Q 1/6825; C12Q 1/6841; C12Q 1/6855; C40B 40/06

(52) U.S. Cl. CPC C12Q 1/6825; C12Q 1/6841; C12Q 1/6855; C40B 40/06

(57) ABSTRACT

Methods for optical in situ nucleotide sequence detection within cells, including mapping multi-modal phenotypes to perturbations in cells and tissues, are provided herein. Such methods provide a sequencing-free barcode readout approach for optical pooled CRISPR screens that is compatible with highly multiplexed antibody and RNA transcript profiling. Compositions comprising barcoded oligonucleotides and detection probes are also provided for use in performing the nucleotide sequence detection methods.

### CROSS-REFERENCE TO RELATED APPLICATIONS

This application claims the benefit of U.S. Provisional Application No. 63/725,319, filed Nov. 26, 2024, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences and/or amino acid sequences which are present in the file named “93597-7429_92319-A_Sequence_Listing_AWG.xml”, which is 16,492 bytes in size, and which was created on Nov. 25, 2025 in the IBM-PC machine format, having an operating system compatibility with MS-Windows, which is contained in the xml file filed Nov. 26, 2025 as part of this application.

### BACKGROUND OF THE INVENTION

Sequencing-based methods which require cell lysis are not amenable to investigating the effects of genetic perturbations on spatial phenotypes, such as cell morphology, protein subcellular localization, cell-cell interactions, and tissue organization. New approaches are needed in order to detect and localize nucleotide sequences in intact cells and probe such phenotypes.

### BRIEF SUMMARY OF THE INVENTION

The invention provides a composition comprising a barcoding oligonucleotide pair, the pair comprising:
a) a single-stranded DNA padlock detection oligonucleotide molecule comprising
   i) a first primer detection oligonucleotide hybridization portion;
   ii) a padlock barcode portion (H1) comprising a nucleotide sequence complementary to a first targeted nucleotide sequence (H1′) of a targeted polynucleotide molecule;
   iii) a first padlock readout portion (rs2);
   iv) a second padlock readout portion (rs1); and
   v) a second primer detection oligonucleotide hybridization portion; and
b) a single-stranded DNA primer detection oligonucleotide molecule comprising
   i) a primer barcode portion (H2) comprising a nucleotide sequence complementary to a second targeted nucleotide sequence (H2′) of the targeted polynucleotide molecule;
   ii) a first padlock detection oligonucleotide hybridization portion comprising a nucleotide sequence complementary to the first primer detection oligonucleotide hybridization portion nucleotide sequence;
   iii) a first primer readout portion (rs4);
   iv) a second primer readout portion (rs3); and
   v) a second padlock detection oligonucleotide hybridization portion comprising a nucleotide sequence complementary to the second primer detection oligonucleotide hybridization portion nucleotide sequence.

The invention also provides a composition comprising a transcript profiling oligonucleotide pair, the pair comprising:
a) a single-stranded DNA padlock detection oligonucleotide molecule comprising
   i) a first primer detection oligonucleotide hybridization portion;
   ii) a padlock transcript-binding portion (H1) comprising a nucleotide sequence complementary to a first targeted nucleotide sequence (H1′) of a targeted polynucleotide molecule;
   iii) a first padlock readout portion (rs1);
   iv) a second padlock readout portion (rs2);
   v) a third padlock readout portion (rs3);
   vi) a second primer detection oligonucleotide hybridization portion; and
b) a single-stranded DNA primer detection oligonucleotide molecule comprising
   i) a primer transcript-binding portion (H2) comprising a nucleotide sequence complementary to a second targeted nucleotide sequence (H2′) of the targeted polynucleotide molecule;
   ii) a first padlock detection oligonucleotide hybridization portion comprising a nucleotide sequence complementary to the first primer detection oligonucleotide hybridization portion nucleotide sequence;
   iii) a splint hybridization primer readout portion (rs4); and
   iv) a second padlock detection oligonucleotide hybridization portion comprising a nucleotide sequence complementary to the second primer detection oligonucleotide hybridization portion nucleotide sequence.

The invention also provides a composition comprising a barcoding oligonucleotide set, the set comprising:
a) a single-stranded DNA left primer oligonucleotide molecule comprising
   i) a left universal bridge oligonucleotide hybridization portion;
   ii) a first left readout portion (rs1);
   iii) a second left readout portion (rs2);
   iv) a left padlock bottom oligonucleotide hybridization portion; and
   v) a left barcode portion (H1) comprising a nucleotide sequence complementary to a first targeted nucleotide sequence (H1′) of a targeted polynucleotide molecule; and
b) a single-stranded DNA padlock bottom oligonucleotide molecule comprising
   i) a right primer oligonucleotide hybridization portion;
   ii) a padlock bottom barcode portion (H2) comprising a nucleotide sequence complementary to a second targeted nucleotide sequence (H2′) of the targeted polynucleotide molecule; and
   iii) a left primer oligonucleotide hybridization portion comprising a nucleotide sequence complementary to the left padlock bottom oligonucleotide hybridization portion of the left primer oligonucleotide; and
c) a single-stranded DNA right primer oligonucleotide molecule comprising
   i) a right barcode portion (H3) comprising a nucleotide sequence complementary to a third targeted nucleotide sequence (H3′) of the targeted polynucleotide molecule nucleotide sequence;
   ii) a right padlock bottom oligonucleotide hybridization portion comprising a nucleotide sequence complementary to the right primer oligonucleotide hybridization portion of the bottom padlock molecule;
   iii) a first right primer readout portion (rs4);
   iv) a second right primer readout portion (rs3); and
   v) a right universal bridge oligonucleotide hybridization portion; and
d) a universal bridge oligonucleotide molecule comprising
   i) a left primer oligonucleotide hybridization portion comprising a nucleotide sequence complementary to the left universal bridge oligonucleotide hybridization portion nucleotide sequence;
   ii) an intervening bridge portion; and
   iii) a right primer oligonucleotide hybridization portion comprising a nucleotide sequence complementary to the right universal bridge oligonucleotide hybridization portion nucleotide sequence
      preferably wherein (d)(i) and (d)(iii) differ in melting temperature,
         preferably such that hybridization of (d)(i) to (a)(i) may occur separately from the hybridization of (d)(iii) to (c)(v) under a first hybridization condition; and
      may occur at the same as hybridization of (d)(iii) to (c)(v) under a second hybridization condition.

### BRIEF DESCRIPTION OF THE DRAWINGS

FIG. 1: Schematic overview of assay. A) A pooled plasmid library is used to generate a lentiviral library. B) Lentiviral library infects target cell line at a low MOI to generate a pool of perturbed cells for which most cells that are infected are only perturbed for a single target. C) In situ multi-omic profiling of perturbed cells by combining cyclic immunofluorescence with highly multiplexed, targeted, mRNA detection, while concurrently reading out the barcode to identify which perturbation a given cell incurred. D) modified CROP-seqV2 plasmid structure, showing PolIII mediated CRISPR guide expression in addition to PolII mediated expression of puromycin transcript that carries the barcode sequence. E) Steps of barcode detection, signal amplification, and signal readout. F) Cyclic imaging to readout barcode identity as defined by a 24 bit (8 cycles, 3 channels) code, uniquely identifying each guide in the pool. G) Barcode detection enables analysis of optically measured phenotypes as a function CRISPR perturbation.

FIG. 2: A) left: unperturbed HT1080 cells expressing copGFP, middle: perturbed cells where some cells still express GFP, whereas other cells have lost GFP expression, right: same perturbed cells, with barcodes identifying if a cell expresses a non-targeting CRISPR guide (magenta) or a GFP targeting CRISPR guide (orange), showing excellent agreement between guide identity and GFP expression status. B) histogram of the number of barcodes detected per cell, red line is the cutoff we choose for further analysis. C) histogram of assigned amplicon purity per cell, where a purity of 1 indicates only a single barcode identity that is recovered under the cell mask. D) Of the amplicons which were positive for 4 out of 24 bits, 98% were allowed primer-padlock probe combinations (orange), whereas 2 percent were unallowed (blue). E) violin plots of GFP fluorescence for unperturbed cells (blue) which resembles cells perturbed by a non-targeting guide (orange), whereas cells with a GFP targeting guide shows significant lower GFP levels (105 is the noise floor). F) CRISPR guide frequency in the pooled library is conserved throughout different stages of lentiviral library preparation.

FIG. 3: Correlation of frequencies of guides observed at different stages of lentiviral library preparation. Guide frequencies are either counted by percentage of sequencing reads for PCR, plasmid and genomic DNA libraries, and by percentage of cells assigned a particular guide.

FIG. 4: Optically read out amplicons are predominantly allowed (orange) combinations between primer and padlock oligos, whereas a minimal amount of unallowed (blue) combinations are read out.

FIG. 5: Boxplots of average GFP fluorescence measured per cell mask for each of the CRISPR guides in the pilot study.

FIG. 6: Schematic outline of genome-wide detection assay.

FIG. 7: Genome-wide assay applied for two (2) different barcodes (red and yellow). Mutually exclusive expression by cells is observed as expected, with a significant number of amplicons per cell (median=8) to distinguish perturbed cells from unperturbed cells.

FIG. 8: Human OE19 cells subcutaneously injected in mice and after 2 weeks of growth tumors were isolated and profiled. Colored spots represent barcode identities detected in tumor cells, red antibody stain is extracellular matrix protein Tenascin, and cyan stain is CD31.

FIG. 9: Multi-omic profiling of CRISPR edited cells in tumor microenvironment A) CRISPR guide identifying barcodes (colored by guide ID). Concurrent multiplexed antibody readout of the tumor microenvironment with visualization of B) vasculature by endothelial marker CD31 (green), C) cell death by cleaved PARP (red), D) extracellular matrix by Tenascin (red).

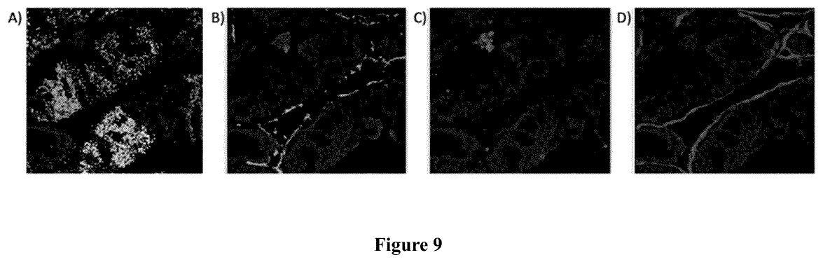

FIG. 10: A) Schematic of validation approach of combinatorial perturbation. B) Boxplot of GFP (left) and RFP (right) intensity of cells when categorized by the EnAsCas12a guide array detected C) Imaging data showing GFP (green) and RFP (red) expression (overlap results in yellow, DAPI in blue) overlayed with barcodes identified in situ (brown dots are NTC-NTC guides, and cyan dots are GFP-RFP targeting guides), showing the expected genotype-phenotype relationship.

FIG. 11: Validation of optical detection of mRNA transcripts in MCF7 cells in multi-omic assay. Left: X-axis:spots per cell for a given transcript, y-axis: mean expression by bulk RNA-Seq. We profiled 12 genes in situ, and observe a Pearson correlation of 0.8.

FIG. 12: A) Multi-omic profiling of perturbed cells. From top left to bottom right: WGA staining for cell segmentation (magenta), cell and nuclei segmentation (magenta and blue respectively), RAD51 antibody stain (red), CYCLIN A2 antibody stain (green), Ubc mRNA transcripts (falsecolored green spots), Ppib mRNA transcripts (falsecolored yellow spots), Hprt1 mRNA transcripts (falsecolored orange spots), Flic (bacterial gene as negative control, falsecolored white spots), barcode identity (falsecolored spots).

FIG. 13: Multi-omic profiling of perturbed cells. Top left to bottom right, from left to right: Cell & nuclei segmentation based on WGA and DAPI stains, falsecolored barcode detection, RPA2 antibody (green), RAD51 antibody stain (red), γH2AX (yellow), Ki67 (purple), CYCLIN A antibody (red), phospo-H3 antibody (yellow), P21 antibody (yellow), p53 antibody (green), IRF3 antibody (yellow), RELA antibody (green).

FIG. 14: mRNA detection in a mouse liver tissue. We recover the expected spatial expression pattern around central veins with pericentral expression of Glul (cyan), and expression of Albumin (red) and Pck1 (green) by hepatocytes. Right: zoomed out montage showing repeated pericentral expression of Glul at central veins.

FIG. 15: left: cell segmented melanoma cells (red dot) and T-cells (yellow or blue dot). Cell masks for melanoma cells are filled by color indicating which gene was perturbed in them. Right: Fold change of normalized cell count for the different perturbed genes to compare a T-cell negative culture to a T-cell positive co-culture experiment.

FIG. 16: Example DNA encoding primer and padlock oligonucleotides. The top strand is SEQ ID NO: 4 and its complementary strand is SEQ ID NO: 5.

FIG. 17: CRISPRmap assay design overview. (A) Synthesized single-guide RNA (sgRNA) and barcode (BC) library are cloned onto the modified CROPseq vector for sgRNA and BC expression. (B) Plasmids are lentivirally transduced into target cells. (C) Design of the CRISPRmap-CROPseq-Guide-Puro vector. Human U6 (hU6) promoter (black) drives the sgRNA expression by RNA Pol III and a Pol III stop signal is inserted between the sgRNA and the barcode. The hU6-sgRNA-stop cassette and the BC are inserted in the 3′ LTR sequence and will thus be copied during genome integration to the upstream of the EF-1a promoter. The EF-1a promoter drives the expression of the CROPseq mRNA by RNA Pol II, which expresses the Puromycin resistance gene (green), hU6 (black), sgRNA (magenta) and BC (cyan). (D) In situ multi-omic phenotyping and CRISPRmap barcode detection. Multi-omic phenotyping interrogates proteomic and transcriptomic states while CRISPRmap barcode readout identifies the sgRNA identity. Cyclic antibody staining (IBEX) is used to detect dozens of epitopes. Pairs of padlock and primer oligos are hybridized to the CROPseq mRNA or endogenous RNAs to detect CRISPRmap barcodes or target RNA transcripts. (E) In situ barcode detection and amplification. Padlock and primer oligos hybridize to the BC sequence on the CROP-seq mRNA. Padlock and primer each encode a unique pair of readout sequences (rs). Splints hybridize to the corresponding rs sequences on primer oligos. Padlock oligos and splints are joined by T4 ligation to enable rolling circle amplification. Fluorophore-conjugated readout probes hybridize to readout sequences on the amplicons in a cyclic manner for barcode identification. (F) Barcode readout and decoding. Images across fluorescence channels and imaging cycles are co-registered into a unified readout stack. Barcode decoding at the amplicon level is achieved through spot detection, assigning a bit code (0 for absence, 1 for presence) in each image to generate a barcode across images. If the barcode aligns with a guide-identifying barcode in the codebook, a guide identity is assigned to the corresponding amplicon. (G) Phenotype-genotype analysis. Multi-omic and multiplexed phenotyping provides high dimensional optical features for systematic analysis.

FIG. 18: CRISPRmap high-fidelity genotype-phenotype mapping. (A) Visualization of genotype-phenotype mapping in cells without (left) or with (middle and right) GFP-pilot library. WGA (magenta) and DAPI (blue) signals are shown (left and middle). Cell boundaries outlined in blue, whereas decoded barcodes are shown as false-colored spots (magenta for GFP-targeting, green for non-targeting guides) (right). Raw GFP signal is displayed by gray-scale in all panels. Scale bar, 50 μm. (B) Visualization of the barcode readout and phenotyping, showing a cell with a GFP-targeting guide (top row) and a cell with a non-targeting guide (bottom row). Decoded barcodes are displayed as white spots (second most right column) and projected onto the 8 readout images as white circles with raw readout signals displayed in magenta for channel 1 and green for channel 2 (column 1 to 8). Raw GFP signal is shown in gray-scale (most right column). Cell and nuclear boundaries outlined in blue in all panels. Scale bar, 10 (top) and 15 (bottom) m. (C) Quantification of all possible primer-padlock combinations, showing robust detection of the 10 allowed combinations and minimal detection of the 15 unallowed combinations. (D) Distribution of the number of assigned amplicons per cell under the standard QC (Methods). (E) Quantification of genotype-phenotype mapping showing cells with GFP-targeting guides have significantly reduced GFP fluorescence (p=1.53e−209). Two-sided Mann-Whitney test, *p<0.05, **p<0.01, ***p<0.001, ****p<0.0001. n=1 with 4620 transduced cells, n=1 with 4810 non-transduced cells. Boxes indicate the median and interquartile range (IQR) with whiskers extending 1.5×IQR past the upper and lower quartiles. (F) Barcode detection between Conventional OPS and CRISPRmap for HT1080, Fibroblast, iPSC, iMotorNeuron and hESC cells. Scale bar, 10 μm. (G) Fraction of cells with barcode detection in CRISPRmap on fibroblast (n=2 biological replicates), HT1080 (n=5 from four biological replicates), iPSC (n=5 from two biological replicates), iMotorNeuron and hESC, and Conventional OPS on fibroblast (n=3 from two biological replicates), HT1080, iPSC, iMotorNeuron and hESC. n=2 technical replicates unless otherwise specified. Data are presented as mean values+/−95% confidence interval (CI).

FIG. 19: CRISPRmap base-editing screening enables multi-omic phenotyping of cell states. (A) Experimental workflow (Methods). Image made in Biorender. (B) Subcellular distribution of 6 DNA damage response protein stains (top row), 5 cell cycle regulator stains (middle row), barcode detection (middle row, most right), and transcript detection for 6 genes (bottom row) for a single cell. Cell and nuclear segmentation outlined in blue, raw antibody signal and transcript detection in gray-scale. Decoded barcodes are shown as false colored (magenta) spots. Only data under the cell segmentation mask is displayed. Scale bar, 10 μm. (C) Quantification of the number of RAD51 foci per cell across cell cycle phases in UNT (n=1 with 120,253 cells) and IR (n=1 with 106,116 cells) cells, showing significant foci induction by irradiation and enrichment in S/G2 phase. Boxes indicate the median and interquartile range (IQR) with whiskers extending 1.5×IQR past the upper and lower quartiles (outliers are omitted). Two-sided Mann-Whitney test, *p<0.05, **p<0.01, ***p<0.001, ****p<0.0001. The p-values of (G2/S_UNT, G0_IR, G1_IR, M_IR) vs. G2/S_IR are 0.00e+00, 0.00e+00, 0.00e+00, 2.74e−37, respectively. (D) as in C) for BRCA1 foci. The p-values are 0.00e+00, 0.00e+00, 0.00e+00, 3.78e−15. (E) Correlation between RNA-reporting spots per million spots measured by RNAmap and Transcript Per Million (TPM) reads from RNA sequencing. Pearson correlation (r) equals 0.84. (F) RNA-protein correlation measured by RNAmap and antibody staining for three RNA-protein pairs (Ccna2-cyclin A2, Ccnb1-cyclin B1, Cdkn1a-p21), showing significantly enriched RNA-reporting spots in cells with high protein expression. Boxes indicate the median and IQR with whiskers extending 1.5×IQR past upper and lower quartiles. Two-sided Mann-Whitney test, *p<0.05, **p<0.01, ***p<0.001, ****p<0.0001. The p-values (from left to right) are 0.00e+00, 0.00e+00, 0.00e+00. (G) Visualization of the decoded Ccna2-reporting spots (magenta) and cyclin A2 staining (green). Scale bar, 50 μm. (H) as in G) for Cdkn1a (magenta) and p21 (green).

FIG. 20: Performance of CRISPRmap base-editing screening on gamma-irradiated MCF7 cells. (A) Correlation between log 2-fold change (L2FC) in RAD51 foci number and the Rule Set 2 on-target score. All guides targeting RAD51 regulators including RAD51 paralogs (RAD51D, RAD51C, XRCC3), BRCA1, and BRCA2 are shown. Splice and nonsense variants with high Rule Set 2 (RS2) score shows more significant L2FC. Pearson correlation (r) equals −0.30. (B) Quantification of RAD51 foci in irradiated S/G2-phase cells with guides targeting RAD51 regulators that have low RS2 score, grouped by sgRNA category. No or moderate significant separation from cells with control guides is observed. Two-sided Kolmogorov-Smirnov (KS) test, *p.adj<0.05, **p.adj<0.01, ***p.adj<0.001, ****p.adj<0.0001. The p-values (from top to bottom) are 4.19e−01, 8.03e−03, 9.79e−01. (C) as in B) for guides with high RS2 score, showing significant reduction in RAD51 foci in cells with nonsense and splice guides. The p-values (from top to bottom) are 9.07e−02, 1.11e−15, 9.82e−17. (D) as in A) for L2FC in BRCA1 foci for BRCA1-targeting guides. Pearson correlation (r) equals −0.75. (E) same as B) for BRCA1 foci and guides targeting BRCA1 that have low RS2 score. The p-values (from top to bottom) are 3.52e−01, 4.99e−01. (F) same as E) for guides with high RS2 score, showing significant reduction in BRCA1 foci in all categories. The p-values (from top to bottom) are 4.88e−03, 1.30e−10, 6.86e−06. (G) Volcano plot showing no AAVS1-targeting or non-targeting control (NTC) guides shows statistically significant changes in RAD51 foci. Guides targeting DDR genes with RS2 score ≥0.55, all AAVS1-targeting and NTC guides are shown. Two-sided KS test. (H) Same as G) highlighting guides that result in significant changes in RAD51 foci. (I) Gene enrichment analysis among guides causing significant changes in RAD51 foci. One-sided (greater) Fisher exact test. Significance, p.adj<0.05. (J) same as G) for BRCA1 foci. (K) same as H) for guides that result in significant changes in BRCA1 foci (L) same as I) for BRCA1 foci.

FIG. 21: Variant analysis on functionally relevant genes identified variant clusters with treatment-specific optical signatures. (A) Crucial effectors in DNA damage repair. RAD51 paralogs including RAD51D, RAD51C and XRCC3 are required for the formation of RAD51 foci at DNA double-strand breaks (DSBs). The BRCA1-BARD1 complex recruits RAD51 to DSB sites. FANCG and FANCI are involved in DNA interstrand crosslinking (ICL) repair. (B) Clustering of guides targeting RAD51 paralog genes (RAD51C, RAD51D, XRCC3), showing a cluster with reduced RAD51 foci in all four DNA-damaging agents-treated and irradiated (IR) cells and increased large γH2AX foci in OLAP- and CISP-treated cells. The mutations of this cluster are mostly splice and nonsense variants. The leftmost column cluster features milder phenotypes mainly associated with missense VUS variants. Log 2-fold change (L2FC) in each optical phenotype in corresponding treatment conditions were shown as rows in the heatmap. Cells in all cell cycle phases are included, untreated cells are not included. All guides with Rule Set 2 on-target score ≥0.5 were included in the clustering and shown in the heatmap. Columns were cut at a depth of 2 and rows were cut at a depth of 3 based on the dendrogram. Color scale is −1 to 1. (C) same as B) for guides targeting BRCA1 and BARD1, showing a cluster with reduced RAD51 and BRCA1 foci in irradiated cells and increased large γH2AX foci and micronuclei in OLAP- and CISP-treated cells composed mainly of pathogenic splice and nonsense variants, and another cluster with mild phenotypes composed mainly of missense variants. (D) same as B) for guides targeting FANCI and FANCG, showing a cluster with mostly splicing variants showing increased large γH2AX foci and micronuclei in OLAP- and CISP-treated cells. The leftmost column cluster features milder phenotypes mainly associated with missense variants.

FIG. 22: Subcellular resolution CRISPRmap barcode readout and multiplexed phenotyping in vivo. (A) Experimental workflow. Cas9 negative OE19 cells were transduced with the 364 guide DDR library and selected with puromycin for 2 days, prior to inoculation in the flank of nude mice. Tumors were harvested after 17 days of growth and processed for CRISPRmap and immunofluorescence imaging. Image made in Biorender. (B) Quantification of proportion of cells segmented on the E-cadherin stain passing the barcode Quality Check (QC) criteria (blue, Methods), and proportion of E-cad segmented cells that is part of a clonal region (orange, Methods). Data are presented as mean values+/−95% CI. n=3 technical replicates. (C) Visualization of in vivo barcode detection, showing the guide distribution landscape in a tumor section. Decoded barcodes are shown as spots, false colored according to their guide identity. The region highlighted by a white dashed square is zoomed in on E-H). Scale bar, 200 μm. (D) Clonality analysis of barcoded cells in a cell-centric manner based on 10 nearest neighbors graphs (methods). Scale bar, 50 μm. (E) Cell (green) and nuclear (blue) boundaries detected by segmentation of E-cadherin and DAPI, respectively. Subcellular resolution of barcode readout. Decoded barcodes are shown as spots, false colored according to their guide identity. (F) Iterative immunofluorescence distinguishes cell types and cellular states in vivo. Protein stains of Tenascin C (magenta) and mouse CD31 (green), and DAPI (blue) are shown. Antibodies are predicted to recognize epitopes from both human and mouse origins unless otherwise specified. (G) as in F) for Vimentin (magenta) and human p21 (green). (H) as in F) for N-cadherin (magenta) and E-cadherin (green).

FIG. 23: Performance of the GFP-targeting pooled optical screen. (A) Genotype-phenotype mapping in cells transduced with the GFP-pilot library. Each GFP-NTC pair was tested, the least significant pair is shown (p=1.84e−10). Two-sided Mann-Whitney test, *p<0.05, **p<0.01, ***p<0.001, *****p<0.0001. n=1 with 4620 cells. Boxes indicate the median and interquartile range (IQR) with whiskers extending 1.5×IQR past the upper and lower quartiles. (B) Performance of Conventional OPS on cells with the GFP-pilot library profiled at the original cell density (n=2, technical replicates, 8104 and 7671 cells), CRISPRmap on cells with GFP-pilot library at the original density (n=1, 4620 cells) and sparsely seeded (n=1, 3429 cells), and CRISPRmap on GFP/mTurquoise2-expressing cells with FP-reporting barcodes (GFP/mTurquoise2-reporting cells) sparsely seeded (n=1, 920 cells). Data are presented as mean values+/−95% CI. (C) Performance of CRISPRmap on cells with GFP-pilot library at the original density under loose to tight QC metrics displayed on the x-axis as (max spot cutoff, purity cutoff). (D) As in C) for CRISPRmap on sparsely seeded cells with GFP-pilot library. (E) As in C) for CRISPRmap on sparsely seeded GFP/mTurquoise2-reporting cells. (F) Genotype-phenotype mapping on GFP/mTurquoise2-reporting cells. Raw mTurquoise2 (left) and GFP (middle) fluorescence are displayed in greyscale. Detected barcodes (right) are shown as spots with cell boundaries outlined in blue. Scale bar, 20 μm. (G) Quantification of the average GFP and mTurquoise2 intensity under each nucleus mask colored by barcode identity. (H) Quantification of the relative guide abundance across PCR-amplified oligo pool (PCR), plasmid pool (Plasmid), genomic DNA (gDNA) of transduced cells, and optical screens (Optical). (I) Pearson correlation of relative guide abundance at PCR, Plasmid, gDNA, and Optical level. (J) Quantification of sgRNA-barcode recombination for the DDR364 and GFP-pilot library at PCR, Plasmid and gDNA level. (K) Visualization of double-transduced cells. Decoded barcodes of the most (magenta) and second most (green) representing guides in each cell are shown as spots. Raw GFP fluorescence is displayed in greyscale. Cell and nuclear boundaries are outlined in blue. Scale bar, 20 μm. (L) Quantification of expected and optically measured ratio of double-transduced cells under different Multiplicity of Infection (MOI). Data are presented as mean values+/−95% CI. n=2, technical replicates for each MOI.

FIG. 24: CRISPRmap application on multiple cell lines. Visualization of barcode detection on five cell lines: A735, U2OS, A549, SW620, and HEK293 (left to right). The morphology of each cell line is shown (top row) with membrane or cytoplasmic stains (blue) and nuclear stain (white). The fluorescence signal from two CRISPRmap imaging cycles are shown in magenta (middle row) and green (bottom row). Cell boundaries are outlined in blue. Scale bar, 50 μm.

FIG. 25: CRISPRmap application on multiple cell lines with cell type validation by immunofluorescence. (A) Visualization of the cell type marker expression (SOX2: stem cell marker, OCT4: stem cell marker, NeuN: neuronal marker) and detected CRISPRmap barcodes (from left to right) on Induced pluripotent stem cells (iPSCs, top row) and iPSC-derived motor neurons (iMotorNeuron, bottom row). Raw fluorescence signal from the cell type markers were displayed in greyscale. Detected CRISPRmap barcodes were shown as false-colored spots with each color corresponding to a particular barcode, and cell boundaries outlined in blue. Scale bar, 20 μm. (B) As in A) for cell type markers (SOX2: stem cell marker, OCT4: stem cell marker, Nanog: stem cell marker) and detected barcodes on human embryonic stem cells (hESCs).

FIG. 26: Conventional optical pooled screen (OPS) application on HT1080 cells transduced with the GFP-pilot library. (A) Visualization of the guide sequence readout by in situ sequencing, showing a cell with a GFP-targeting guide (“TGAAGATCACGCTGTCCTCG”). Raw fluorescence signal over four readout rounds and three fluorescence channels are displayed in greyscale. Scale bar, 20 μm. (B) As in A) for decoded reads, which are displayed as false-colored spots. Cell and nucleus boundaries are outlined in blue. (C) As in A) for raw GFP fluorescence shown in gray-scale.

FIG. 27: Multi-modal profiling of base-edited cells at single-cell resolution. (A) The subcellular distribution of 13 protein stains (top row), 12 transcript detection for 12 genes (bottom row, starting from the most left) and barcode detection (bottom row, most right), are shown for a single cell with the AAVS1.100 guide. Cell and nuclear segmentation are outlined in green, whereas raw antibody signal and transcript detection are in gray-scale. Decoded barcodes are shown as false colored (magenta) spots. Scale bar, 5 μm. (B) As in A) for a cell with the BRCA1.416 (H1283Y) guide. (C) As in A) for a cell with the BRCA1.308 (Q380*) guide. (D) As in A) for a cell with the RAD51D.1 (splice) guide. Only raw data under the cell mask of the cell of interest is plotted.

FIG. 28: Multiplexed immunofluorescence enables cell cycle phase separation and nuclear foci detection. (A) Visualization of nuclear foci detection for RAD51. Raw antibody stain for RAD51 is shown in grayscale in both left and right panels. Nuclear boundaries outlined in blue. All computationally detected RAD51 foci are displayed as circles in false color (magenta) on the right panel. Scale bar, 20 μm. (B) Same as A) for γH2AX. All computationally detected γH2AX foci are displayed as circles in false color (magenta) on the right panel, whereas computationally detected spots categorized as large γH2AX foci are displayed as circles of false color green. The same region in A) is shown. Scale bar, 20 μm. (C) Quantification of the number of RPA2 foci per cell across cell cycle phases in untreated (UNT) (n=1 with 120,253 cells) and irradiated (IR) cells (n=1 with 106,116 cells). Boxes indicate the median and interquartile range (IQR) with whiskers extending 1.5×IQR past the upper and lower quartiles (outliers are omitted). Two-sided Mann-Whitney test, *p<0.05, **p<0.01, ***p<0.001, ****p<0.0001. Exact p-values of G1UNT vs. G1IR, G0UNT vs. G0IR, G2/SUNT vs. G2/SIR, MUNT vs. MIR, G2/SIR vs. MIR, G1IR vs. G2/SIR, G0IR vs. G2/SIR are 0.00e+00, 0.00e+00, 0.00e+00, 2.95e−19, 7.13e−08, 0.00e+00, 0.00e+00, respectively. (D) Same as C) for RAD18 foci. p-values are 0.00e+00, 3.17e−77, 0.00e+00, 1.15e−01, 3.49e−15, 0.00e+00, 0.00e+00. (E) Same as C) for large γH2AX foci. p-values are 0.00e+00, 0.00e+00, 0.00e+00, 2.53e−36, 2.80e−03, 0.00e+00, 1.32e−124. (F) Same as C) for large 53BP1 foci. pvalues are 0.00e+00, 5.17e−08, 0.00e+00, 3.04e−28, 8.59e−06, 0.00e+00, 1.01e−21. (G) Quantification of protein expression level of cyclin A2, cyclinB1, and p21. A threshold on the average fluorescence intensity under nuclear masks is set to categorize the expression level of the corresponding protein into low (smaller than the threshold) or high (greater than or equal to the threshold) categories. The threshold for each protein is displayed as a dashed line on the corresponding histogram. (H) Correlation of optical features measured in this experiment, showing positive correlation among nuclear foci involved in homologous recombination (RAD51, BRCA1, RPA2, RAD18), S/G2 phase cell cycle markers (cyclin A2, cyclin B1) and proliferation marker (Ki-67).

FIG. 29: Library representation in the CRISPRmap base-editing screening on MCF7 cells treated with ionizing radiation or DNA damaging agents. (A) Correlation of the relative abundance of guides in the DDR364 library based on the percentage of sequencing reads in plasmid library and the percentage of barcode-assigned cells in the optical screen under the camptothecin (CPT) treatment. Pearson correlation (r) is 0.67. Line, linear regression fit; shaded area, 95% confidence interval of the fit. (B) As in A) for cells under olaparib (OLAP) treatment. Pearson correlation (r) is 0.63. (C) As in A) for cells under cisplatin (CISP) treatment. Pearson correlation (r) is 0.66. (D) As in A) for cells under etoposide (ETOP) treatment. Pearson correlation (r) is 0.66. (E) As in A) for untreated cells (UNT1) as the control for the four DNA damaging agents in A)-D). Pearson correlation (r) is 0.67. (F) As in A) for cells under ionizing radiation (IR). Pearson correlation (r) is 0.63. (G) As in A) for untreated cells (UNT2) as the control for the IR-treated cells in F). Pearson correlation (r) is 0.65. (H) Visualization of the Pearson correlation among the relative guide abundance in cells treated under the seven conditions in A)-G) and the plasmid library.

FIG. 30: Performance of CRISPRmap base-editing screening on MCF7 cells treated with ionizing radiation or DNA damaging agents. (A) Wasserstein distance of cells with DDR gene-targeting guides (Perturb) or control guides (Controls) to control cells for RAD51 foci in irradiated cells. Hits of RAD51 foci identified in the pooled screening are marked. (B) same as A) for BRCA1 foci. (C) Volcano plots showing variants yielding significant changes in the proportion of untreated (left) or irradiated (right) cells with high p21 expression. Significance, p.adj<0.05. Two-sided Beta-Binomial test. The proportion of control cells with high p21 is marked with the vertical line. (D) Experimental workflow (Methods). Image made in Biorender. (E) Correlation between L2FC in RAD51 foci in CISP-treated cells and the Rule Set 2 (RS2) score. All guides targeting RAD51D, RAD51C, XRCC3, BRCA1, and BRCA2 are shown. The Pearson correlation (r) equals 0.29. (F) same as E) for OLAP-treated cells. The Pearson correlation (r) equals 0.30. (G) Volcano plot showing variants yielding significant changes in RAD51 foci in CISP-treated cells. Guides targeting DDR genes with RS2 score >=0.5, all AAVS1-targeting and non-targeting control (NTC) guides are shown. Two-sided KS test, *p.adj<0.05, **p.adj<0.01, ***p.adj<0.001, ****p.adj<0.0001. Figure legend as in C). (H) same as G) for OLAP-treated cells. (I) Gene enrichment analysis in variants that result in significant changes in RAD51 foci. One-sided (greater) Fisher exact test. Significance, p.adj<0.05. (J) Same as G) for large γH2AX foci in CISP-treated cells. (K) same as J) for OLAP-treated cells. (L) same as I) for large γH2AX foci. (M) Quantification of the number of optical features with statistical significance scored by variants from different ClinVar categories. Boxes indicate the median and IQR with whiskers extending 1.5×IQR past the upper and lower quartiles. Two-sided Mann-Whitney test, *p<0.05, **p<0.01, ***p<0.001, ****p<0.0001. The p-values (from left to right) are 2.28e−01, 3.69e−03, 3.83e−09, 2.07e−11. (N) Comparison of the Pearson correlation of L2FC in optical features between guides leading to the same amino acid (AA) change and different AA changes (p=4.48e−02). Boxes as in M). Two-sided independent t-test, *p.adj<0.05, **p.adj<0.01, ***p.adj<0.001, ****p.adj<0.0001.

FIG. 31: Foci number distribution and random sampling test of variants resulting in significant loss of RAD51 or BRCA1 foci in the MCF7 cells treated with ionizing radiation. (A) Quantification of the RAD51 foci count in cells with the BRCA2.207 (Q2580*), BRCA2.26 (Q373*), BRCA1.234 (splice), and BRCA1.308 (Q380*) variant (left to right), showing significantly reduced RAD51 foci compared to the cells with AAVS1-targeting and non-targeting (control) variants. Cells in S/G2-phase treated with ionizing radiation are shown. Two-sided KS test, *p.adj<0.05, **p.adj<0.01, ***p.adj<0.001, ****p.adj<0.0001. The p-values (from left to right) are 2.55e−15, 2.70e−12, 1.19e−06, 5.71e−07. (B) Quantification of the sample size effect for the four variants in A) on the statistical significance in RAD51 foci changes. 10 random samples were taken from the screen data for each variant at each sample size and the p-value was calculated by the two-sided K-S test, showing higher statistical significance in all four variants at larger sizes of random samples. P-value cutoff of 0.05 is shown in grey (dashed). Data are presented as mean values+/−95% CI. n=10 technical replicates. (C) As in A) for the BRCA1 foci count in cells with the BRCA1.234 (splice), BRCA1.308 (Q380*), BRCA1.416 (H1283Y), and BARD1.24 (D94N) variant. The p-values (from left to right) are 5.12e−09, 2.39e−08, 4.88e−03, 3.71e−06. (D) As in B) for the four variants in C) on the statistical significance in BRCA1 foci changes.

FIG. 32: Phenotypic validation of hits identified in the pooled screen on MCF7 cells with individually transduced guides. Visualization of the RAD51, BRCA1 and γH2AX antibody staining (left to right) in MCF7 cells individually transduced with the AAVS1.28, AAVS1.86, BRCA1.234 (splice), BRCA1.416 (H1283Y) and BRCA2.207 (Q2580*) guide (top to bottom) after the treatment of ionizing radiation. DAPI staining is shown on the right most column. Raw fluorescence signal of RAD51, BRCA1, γH2AX and DAPI are shown in greyscale. Scale bar, 20 μm.

FIG. 33: Validation of optical phenotypes captured in the pooled screen on MCF7 cells with individually transduced guides. (A) Quantification of the percentage of cells with >5 RAD51 foci in cells that were individually transduced with each of the nine guides and treated by ionizing irradiation. Hits identified in the screen are marked in red, showing significantly lower ratios of cells with >5 RAD51 foci compared to the ratios in cells with the AAVS1 control guides. Data are presented as mean values+/−95% CI. n=3 biological replicates. Two-sided independent t-test, *p.adj<0.05, **p.adj<0.01, ***p.adj<0.001, ****p.adj<0.0001. The exact p-values (from left to right) are 1.95e−02, 1.34e−05, 7.57e−05, 6.21e−06, 1.01e−01, 1.40e−03, 4.88e−01, respectively. (B) As in A) for BRCA1 foci. The exact p-values (from left to right) are 1.201e−02, 6.13e−05, 1.85e−03, 2.32e−03, 6.18e−01, 1.10e−01, 5.47e−01, respectively. (C) Correlation of RAD51 foci log 2-fold change (L2FC) resulted from guides delivered in the pooled screen and guides transduced individually (individual). Hits identified in the pooled screen are marked in red. The Pearson correlation (r) is 0.90. (D) same as C) for BRCA1 foci. The Pearson correlation (r) is 0.95.

FIG. 34: Genomic and protein-level validation of hits identified in the pooled screen on MCF7 cells with individually transduced guides. (A) Quantification of the base-editing efficiency in MCF7 cells individually transduced with the AAVS1.28 (n=2, biological replicates), AAVS1.86 (n=2, biological replicates), BRCA1.234 (splice) (n=2, biological replicates), BRCA1.416 (H1283Y) (n=2, biological replicates) and BRCA2.207 (Q2580*) (n=3, biological replicates) guide (left to right) based on sanger sequencing of the PCR-amplified genomic locus targeted by each guide. Editing efficiency on C bases both inside (in-window) and outside (out-of-window) of the expected editing window were quantified, showing good on-target (inwindow) base editing. The scale of editing efficiency is 0 (no editing) to 1 (complete editing). The C base position is listed as the n-th base from the first base of the gRNA targeted sequence. Data are presented as mean values+/−95% CI. (B) Immunoblots on guides targeting BRCA1 showing the reduction of full-length BRCA1 protein in the BRCA1.234 (splice) variant, compared to the missense variants BRCA1.416 (H1283Y), and the AAVS1.86 variant. Cells transduced with Firefly siRNA (siFirefly) and BRCA1 siRNA (siBRCA1) were included to show the specificity of BRCA1 protein detection. Cells were treated with or without irradiation and the induction of DNA damage is shown with the phospho-KAP1 (pKAP1) staining. Tubulin is used as the loading control. (C) As in B) for BRCA2 variants showing the reduction of full-length BRCA2 protein in the nonsense variant BRCA2.207 (Q2580*), compared to the missense variant BRCA2.438 (A1847T) and the AAVS1.86 variant.

FIG. 35: Variant analysis of functionally relevant genes and variant clusters with treatment-specific optical signatures. (A) Clustering of guides targeting BRCA2 and PALB2, showing a cluster of pathogenic nonsense variants showing a string reduction of RAD51 across all treatments and another cluster showing reduction of RAD51 in CPT-, OLAP-, and CISP-treated cells mainly composed of pathogenic splice variants. Other clusters show mild phenotypes. Log 2-fold change (L2FC) in each optical phenotype in corresponding treatment conditions are shown as rows in the heatmap. Cells in all cell cycle phases are included, untreated cells are not included. All guides with on-target score ≥0.5 were included in the clustering and shown in the heatmap. Columns were cut at a depth of 2 and rows were cut a depth of 3 based on the dendrogram. Color scale is −1 to 1. (B) same as A) for guides targeting ATM, showing a cluster with reduced large γH2AX foci and micronuclei in all treatments, increased micronuclei in CPT-treated cells and increased RAD51 foci in ETOP-treated cells composed of splice variants, and other clusters with milder phenotypes. (C) same as A) for guides targeting all genes in Fanconi anemia (FA) pathway, showing a cluster with increased large γH2AX foci in OLAP- and CISP-treated cells, increased micronuclei in ETOP-treated cells and increased RAD51 foci in ETOP-treated cells, composed of most splice and nonsense variants targeting these genes. The other cluster composed of mainly missense variants shows mild phenotypes.

FIG. 36: Variant analysis of clustering at the library level shows local structure of enrichment in gene targets. Clustering of all targeting guides with a Rule Set2 on-target score (RS2) >0.5 and the non-targeting control guides (273 guides in total) on foci features across five treatments, showing local structures of enrichment in guides targeting the same (groups of) genes. Guides (rows) are colored by their gene targets. RAD51 paralogs (RAD51D, RAD51C and XRCC3) are considered as a group (“RAD51 paralogs”) and FANC family genes (FANCA, FANCC, FANCD2, FANCE, FANCF, FANCG, FANCI, FANCL, FANCM) are grouped as “FANC family”.

FIG. 37: Variant analysis of clustering at the library level identifies clusters with local structure of enrichment in gene targets. Enlarged view of the top 3 clusters of FIG. 36. The top most cluster shows a significant enrichment of guides targeting RAD51 paralogs (p=1.1e−05, hypergeometric test), featured by downregulation of RAD51 foci across all treatments and upregulation of γH2AX foci in CISP- and OLAP-treated cells. Strong enrichment of ATM variants is observed by the second top cluster (p=5.4e−06), where γH2AX foci are significantly reduced in ETOP-treated cells and mostly down-regulated in CISP- and OLAP-treated cells. FANC family gene variants are observed to be enriched in the third top cluster (p=3.2e−05) which features an upregulation of γH2AX foci in OLAP- and CISP-treated cells.

FIG. 38: Colocalization of DNA Damage Repair proteins across cell cycle. (A) Quantification of the number of colocalized DNA Damage Repair (DDR) foci marker per nuclei is significantly higher than random chance colocalization of DDR foci markers per nuclei. Two-sided paired student's t-test, *p<0.05, **p<0.01, ***p<0.001, *****p<0.0001. Boxes indicate the median and interquartile range (IQR) with whiskers extending 1.5×IQR past the upper and lower quartiles. The p-values (from left to right) are 1.61e−277, 9.83e−272, 0.00e+00, 1.05e−291, 0.00e+00 (n=10000 iterations for each condition). (B) Quantification of the number of specific colocalized DDR foci per cell across cell cycle in irradiated (IR) cells. Two-sided independent student's t-test, *p<0.05, **p<0.01, ***p<0.001, *****p<0.0001. (Outliers are omitted in the plot.) The p-values (from left to right) are 0.00e+00, 2.33e−269, 7.15e−245, 1.79e−234, 1.18e−25. n=1 with 106,116 cells. (C) Proportion of DDR foci marker colocalized with another DDR foci marker for S/G2 phase shows strong colocalization of RAD51-BRCA1, RAD51-γH2AX, BRCA1-γH2AX, 53BP1-γH2AX highlighting the prevalence of Non-Homologous End Joining and Homologous Recombination. (D) Proportion of DDR foci marker colocalized with another DDR foci marker for G1 phase shows strong colocalization of 53BP1-γH2AX highlighting the prevalence of Non-Homologous End Joining in G1 phase.

FIG. 39: Quantitative analysis on CRISPRmap barcode readout in tissue. (A) Interaction matrix among guides detected on tissue samples, showing mostly homotypic interactions. All guides with at least 10 cells are shown, and enrichment z scores of interactions are displayed. (B) Visualization of the relative abundance of guides in the DDR364 library based on the percentage of sequencing reads in plasmid library (bottom) and the stacked percentage of barcode-assigned cells in three tissue samples (top). (C) Correlation of the relative abundance of guides in the DDR364 library based on sequencing reads in plasmid library and barcode-assigned cells in tissue samples. Line, linear regression fit; shaded area, 95% confidence interval of the fit. The Pearson correlation (r) is 0.12, 0.13, 0.14, respectively. (D) Estimated tissue area required for a CRISPRmap screen of varying library sizes (50, 100, 150 guides) at different targets of cells passed Quality Check (QC) per guide. Three estimations were made for each target cell number per guide for a given library size based on three tissue samples. Line, linear regression fit; shaded area, 95% confidence interval of the fit. (E) As in D) for different targets of clonal cells passed QC per guide. (F) Visualization of the void regions on the tissue section, showing regions associated with cell death marked by cleaved PARP (displayed in yellow) and regions associated with mouse vasculature marked by CD31 (shown in cyan). Green regions are identified as an overlap of vasculature and cell death. Unmarked regions are not classified into either category. Raw cleaved PARP, CD31 and E-cadherin fluorescence are shown in yellow, cyan and red, respectively. Scale bar, 200 μm. (G) Visualization of the overlay of DAPI fluorescence between the first (Round 1) and the last (Round 12) imaging round on a tissue section, showing minimal tissue loss through the 12 rounds of cyclic CRISPRmap barcode readout and antibody staining. Round 1 and Round 12 DAPI signal is displayed in cyan and red, respectively. The overlap between Round 1 and Round 12 DAPI signal is displayed in white. Scale bar, 200 μm.

### DETAILED DESCRIPTION OF THE INVENTION

The invention provides a composition comprising a barcoding oligonucleotide pair, the pair comprising:
a) a single-stranded DNA padlock detection oligonucleotide molecule comprising
   i) a first primer detection oligonucleotide hybridization portion;
   ii) a padlock barcode portion (H1) comprising a nucleotide sequence complementary to a first targeted nucleotide sequence (H1′) of a targeted polynucleotide molecule;
   iii) a first padlock readout portion (rs2);
   iv) a second padlock readout portion (rs1); and
   v) a second primer detection oligonucleotide hybridization portion; and
b) a single-stranded DNA primer detection oligonucleotide molecule comprising
   i) a primer barcode portion (H2) comprising a nucleotide sequence complementary to a second targeted nucleotide sequence (H2′) of the targeted polynucleotide molecule;
   ii) a first padlock detection oligonucleotide hybridization portion comprising a nucleotide sequence complementary to the first primer detection oligonucleotide hybridization portion nucleotide sequence;
   iii) a first primer readout portion (rs4);
   iv) a second primer readout portion (rs3); and
   v) a second padlock detection oligonucleotide hybridization portion comprising a nucleotide sequence complementary to the second primer detection oligonucleotide hybridization portion nucleotide sequence.

In some embodiments, (a)(i) to (a)(v) of the padlock detection oligonucleotide molecule are arranged 5′ to 3′, respectively.

In some embodiments, the padlock detection oligonucleotide molecule comprises an intervening sequence portion between (a)(i) and (a)(ii), between (a)(ii) and (a)(iii), between (a)(iii) and (a)(iv), and/or between (a)(iv) and (a)(v),
preferably wherein a first intervening sequence portion is between (a)(ii) and (a)(iii), and a second intervening sequence portion is between (a)(iv) and (a)(v), preferably wherein the first and second intervening sequence portions are each about 10-30 nucleotides in length,
and/or comprises an additional sequence portion upstream and/or downstream of any one of (a)(i) or (a)(v).

In some embodiments, (b)(i) to (b)(v) of the primer detection oligonucleotide molecule are arranged 5′ to 3′, respectively.

In some embodiments, the primer detection oligonucleotide molecule comprises an intervening sequence portion between (b)(i) and (b)(ii), between (b)(ii) and (b)(iii), between (b)(iii) and (b)(iv), and/or between (b)(iv) and (b)(v) and/or comprises an additional sequence portion upstream and/or downstream of any one of (b)(i) or (b)(v).

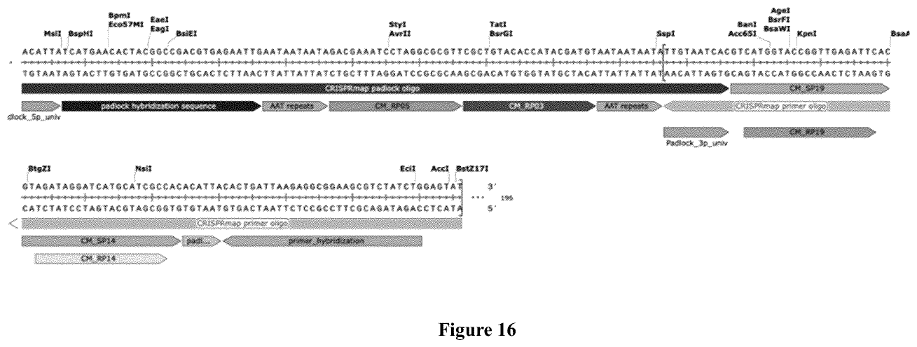

In some embodiments, ‘linker’ sequences that are universal among the padlock probes. (e.g., between H1 and rs1, and between rs2 and the sequence that hybridizes to the primer oligo). This is concept also applies to the ‘transcript detecting oligos’ (e.g., see the “AAT repeats” in FIG. 16).

In some embodiments, H1, H1′, H2, H2′ are 20-50 nucleotides in length, preferably about 30 nucleotides in length,
rs1, rs2, rs3, and/or rs4 are 15-30 nucleotides in length, preferably about 20-25 nucleotides in length, more preferably about 20 nucleotides in length;
the padlock detection oligonucleotide molecule is 50-200, more preferably 50-150, more preferably 75-125 nucleotides in length, more preferably 100-110 nucleotides in length, more preferably about 106 nucleotides in length; and/or
the primer detection oligonucleotide molecule is 50-200, more preferably 50-150, more preferably 50-100 nucleotides in length.

In some embodiments, H1′ and H2′ are separated by no more than 10 nucleotides from each other on the targeted molecule, preferably wherein H1′ and H2′ are adjacent to each other.

In some embodiments, (a) the single-stranded DNA padlock detection oligonucleotide molecule and the (b) single-stranded DNA primer detection oligonucleotide molecule are not covalently bonded to each other.

In some embodiments, the composition further comprises a first single-stranded DNA oligonucleotide splint molecule comprising a nucleotide sequence complementary to the first primer readout portion (rs4) nucleotide sequence.

In some embodiments, the first splint molecule is 15-30 nucleotides in length, preferably about 20-25 nucleotides in length, more preferably 24 nucleotides in length, more preferably wherein the first splint molecule comprises a central splint hybridization portion consisting of a nucleotide sequence complementary to the first primer readout portion (rs4) flanked by additional nucleotides on each end, preferably wherein the central hybridization portion is about 20 nucleotides in length and is flanked by about 2 nucleotides on each end.

In some embodiments, the composition further comprises a second single-stranded DNA oligonucleotide splint molecule comprising a nucleotide sequence complementary to the first primer readout portion (rs3) nucleotide sequence.

In some embodiments, the second splint molecule is 15-30 nucleotides in length, preferably about 20-25 nucleotides in length, more preferably 24 nucleotides in length, more preferably wherein the second splint molecule comprises a central splint hybridization portion consisting of a nucleotide sequence complementary to the first primer readout portion (rs3) flanked by additional nucleotides on each end, preferably wherein the central hybridization portion is about 20 nucleotides in length and is flanked by about 2 nucleotides on each end.

In some embodiments, the composition further comprises a circularized amplicon template oligonucleotide molecule formed by ligation of one, two, or more single-stranded DNA oligonucleotide splint molecules to the padlock detection oligonucleotide molecule.

In some embodiments, the composition comprises one to four single-stranded DNA oligonucleotide probe molecules,
wherein a probe molecule of the one to four single-stranded DNA oligonucleotide probe molecules is conjugated to a detection molecule and comprises an amplicon hybridization portion;
wherein the amplicon hybridization portion of a probe molecule of the one to four single-stranded DNA oligonucleotide probe molecules consists of
i) a nucleotide sequence complementary to the first padlock readout portion (rs2) nucleotide sequence;
ii) a nucleotide sequence complementary to the second padlock readout portion (rs1) nucleotide sequence;
iii) the first primer readout portion (rs4) nucleotide sequence; or
iv) the second primer readout portion (rs3) nucleotide sequence.

In some embodiments, a composition comprises a plurality of unique padlock detection oligonucleotide and primer detection oligonucleotide pairs, with each pair in the plurality having a unique set of readout probes. As a non-limiting example, each pair is defined by a unique set of four readout probes such that the plurality of has 24 readout probes coding four 2916 barcodes, however, this number can be extended by adding additional unique padlock detection oligonucleotide and primer detection oligonucleotide pairs which correspond to more read out sequences.

In some embodiments, the detection molecule is a fluorescent dye, Raman spectroscopy dye, a horse-radish peroxidase (HRP) enzyme, a depositing dye, or a mass spectroscopy imaging isotope.

In some embodiments, the detection molecule of at least one probe molecule has a different emission wavelength maxima than the detection molecule of another probe molecule of the composition, preferably such that the detection molecule of each probe molecule has a different emission wavelength maxima than the detection molecule of any other probe molecule of the composition.

In some embodiments, the composition comprises two to four probe molecules, wherein the amplicon hybridization portion of one probe molecule differs in sequence from the amplicon hybridization portion of another probe molecule, preferably wherein each probe molecule comprises an amplicon hybridization portion that differs in sequence from the amplicon hybridization portion of another probe molecule and is unique among the two to four probe molecules.

In some embodiments, the targeted polynucleotide molecule is an RNA molecule.

In some embodiments, the targeted polynucleotide molecule encodes a spacer sequence portion of a CRISPR guide RNA molecule.

In some embodiments, the composition further comprises a DNA polynucleotide encoding the targeted polynucleotide molecule.

The invention also provides a kit comprising any one of the compositions described herein, optionally further comprising a DNA ligase and/or a DNA polymerase.

The invention also provides a method of detecting a polynucleotide molecule in a cell, the method comprising:
a) delivering to the cell a composition comprising a barcoding oligonucleotide pair described herein;
b) hybridizing the padlock detection molecule and the primer detection molecule to the polynucleotide molecule, and hybridizing the padlock detection molecule to the primer detection molecule;
c) delivering to the cell
   i) a first single-stranded DNA oligonucleotide splint molecule comprising a nucleotide sequence complementary to the first primer readout portion (rs4) nucleotide sequence as described herein; and
   ii) a second single-stranded DNA oligonucleotide splint molecule comprising a nucleotide sequence complementary to the first primer readout portion (rs3) nucleotide sequence as described herein;
d) hybridizing the first splint molecule to the primer detection molecule and hybridizing the second splint molecule to the primer detection molecule;
e) ligating the first and second splint molecules to the hybridized padlock detection molecule to form a circularized amplicon template oligonucleotide molecule;
f) amplifying the circularized amplicon template oligonucleotide molecule by rolling circle amplification to form an amplicon molecule;
g) delivering to the cell one to four single-stranded DNA oligonucleotide probe molecules described herein,
   wherein each probe molecule of the one to four single-stranded DNA oligonucleotide probe molecules is conjugated to a detection molecule and comprises an amplicon hybridization portion;
   wherein the amplicon hybridization portion comprises a sequence selected from
      i) a nucleotide sequence complementary to the first padlock readout portion (rs2) nucleotide sequence;
      ii) a nucleotide sequence complementary to the second padlock readout portion (rs1) nucleotide sequence;
      iii) the first primer readout portion (rs4) nucleotide sequence; or
      iv) the second primer readout portion (rs3) nucleotide sequence; and
h) optically detecting the presence of the polynucleotide molecule in the cell upon hybridization of a probe molecule to the amplicon molecule, preferably optically detecting the presence of the polynucleotide molecule in the cell upon hybridization of each probe molecule delivered to the cell to the amplicon molecule.

In some embodiments, a plurality of a plurality of unique padlock detection oligonucleotide and primer detection oligonucleotide pairs are delivered to a cell, and the cell is exposed to multiple readout probes at a time over multiple cycles (e.g., three readout probes at a time over 8 cycles, yielding the 24 readout probes). Only pairs that formed an amplicon have probes hybridized to them in any given cell. In some embodiments, each readout probe per cycle carries a different signal, e.g., the three readout probes per imaging cycles each carry a different dye such as 488, 560, 640 excitable dyes.

In some embodiments, the one to four single-stranded DNA oligonucleotide probe molecules are delivered at about the same time or at different times.

In some embodiments, in step (g) a first probe molecule of the one to four probe molecules is delivered to the cell, step (h) is performed, and further comprising
i) removing the first probe molecule from the cell;
j) delivering to the cell a second single-stranded DNA oligonucleotide probe molecule described herein; and
k) optically detecting the presence of the polynucleotide molecule in the cell upon hybridization of the second probe molecule to the amplicon molecule.

In some embodiments, the method further comprises
l) removing the second probe molecule of from the cell;
m) delivering to the cell a third single-stranded DNA oligonucleotide probe molecule described herein; and
n) optically detecting the presence of the polynucleotide molecule in the cell upon hybridization of the third probe molecule to the amplicon molecule.

In some embodiments, the method further comprises
o) removing the third probe molecule from the cell;
p) delivering to the cell a fourth single-stranded DNA oligonucleotide probe molecule described herein; and
q) optically detecting the presence of the polynucleotide molecule in the cell upon hybridization of the second probe molecule to the amplicon molecule.

In some embodiments, the cells are fixed cells, and preferably permeabilized cells, and are preferably on a glass bottom well plate surface.

The invention also provides a cellular barcoding library, the library comprising a plurality of barcoding oligonucleotide pairs described herein,
wherein each barcoding oligonucleotide pair comprises a combined readout set of rs1, rs2, rs3 and rs4 sequences;
wherein each combined readout set is unique to its barcoding oligonucleotide pair within the library.

In some embodiments, each first primer detection oligonucleotide hybridization portion is a universal sequence common to each padlock detection oligonucleotide molecule;
each second primer detection oligonucleotide hybridization portion is a universal sequence common to each padlock detection oligonucleotide molecule;
each first padlock detection oligonucleotide hybridization portion is a universal sequence common to each primer detection oligonucleotide molecule; and/or
each second padlock detection oligonucleotide hybridization portion is a universal sequence common to each primer detection oligonucleotide molecule.

In some embodiments, each targeted molecule in a library of targeted molecules comprises a unique targeted portion comprising a first targeted nucleotide sequence (H1′) and a second targeted nucleotide sequence (H2′),
(a) wherein the first targeted nucleotide sequence (H1′) is complementary to a padlock barcode portion (H1) of a padlock detection molecule of a barcoding oligonucleotide pair in the library; and
(b) wherein the second targeted nucleotide sequence (H2′) is complementary to the primer barcode portion (H2) of a primer detection molecule of the said barcoding oligonucleotide pair in (a);
such that no two targeted molecules in the library of targeted molecules share the same unique targeted portion.

In some embodiments, each barcoding oligonucleotide pair is capable of hybridizing to only one targeted molecule in a library of targeted molecules while the padlock detection oligonucleotide molecule and the primer detection oligonucleotide molecule of the pair are simultaneously hybridized.

In some embodiments, the cellular barcoding library further comprises a plurality of single-stranded DNA oligonucleotide splint molecules, wherein each splint molecule comprises a nucleotide sequence complementary to a first primer readout portion (rs4) nucleotide sequence of a primer detection oligonucleotide molecule in the cellular barcoding library.

In some embodiments, the cellular barcoding library further comprises a second plurality of single-stranded DNA oligonucleotide splint molecules, wherein each splint molecule in the second plurality, comprises a nucleotide sequence complementary to a second primer readout portion (rs3) nucleotide sequence of a primer detection oligonucleotide molecule in the cellular barcoding library.

In some embodiments, the cellular barcoding library further comprises a plurality of DNA oligonucleotide probe molecules,
wherein each probe molecule is conjugated to a detection molecule and comprises an amplicon hybridization portion;
wherein the amplicon hybridization portion comprises
   i) a nucleotide sequence complementary to a first padlock readout portion (rs2) nucleotide sequence of a padlock detection molecule of the cellular barcoding library;
   ii) a nucleotide sequence complementary to a second padlock readout portion (rs1) nucleotide sequence of a padlock detection molecule of the cellular barcoding library;
   iii) a first primer readout portion (rs4) nucleotide sequence of a primer detection molecule of the cellular barcoding library; or
   iv) a second primer readout portion (rs3) nucleotide sequence of a primer detection molecule of the cellular barcoding library.

In some embodiments, the number of unique amplicon hybridization portion sequences of the probe molecules in the plurality is at least equal to the number of unique sequences among the totality of combined readout sets of the oligonucleotide pairs within the library.

In some embodiments, the detection molecule is a fluorescent dye or Raman spectroscopy dye.

The invention also provides a method of detecting a polynucleotide molecule from a library of targeted molecules in a cell, the method comprising
(a) delivering a library of targeted molecules to a plurality of cells, preferably such that each cell in the plurality is delivered a targeted molecule;
(b) delivering the cellular barcoding library described herein;
(c) hybridizing the padlock detection molecule and the primer detection molecule of each barcoded oligonucleotide pair to a targeted polynucleotide molecule present in a cell, and hybridizing the padlock detection molecule to the primer detection molecule of each barcoded oligonucleotide pair;
(d) delivering the plurality of splint molecules described herein and the second plurality of splint molecules described herein;
(e) ligating a first and a second splint molecule to a hybridized padlock detection molecule to form circularized amplicon template oligonucleotide molecules;
(f) amplifying each circularized amplicon template oligonucleotide molecule by rolling circle amplification to form amplicon molecules;
(g) delivering a first group of probe molecules from the plurality of single-stranded DNA oligonucleotide probe molecules described herein,
   wherein each probe molecule in the group comprises a unique amplicon hybridization portion which is different in sequence from the other probe molecules in the group;
   preferably wherein the number of probe molecules delivered in the group is equal to the number of probes that can be separately detected or spectrally distinguished in a readout cycle, preferably at most equal to a number of channels on a detection module, preferably a microscope to be used to optically view the cells, preferably wherein the number of probe molecules delivered in the group is 3 probe molecules;
   preferably wherein each probe molecule in the group is conjugated to a detection molecule with a different emission wavelength maxima than the other probe molecules in the group,
      more preferably wherein each probe molecule in the group is conjugated to a detection molecule having an emission wavelength maxima specific to only one channel of the microscope and no two detection molecules have an emission wavelength maxima in the same channel;
(h) optically detecting the hybridization of the probe molecules from the first group to any amplicon molecule in any cell of the plurality of cells;
(i) for each amplicon location,
   mark “1” for each channel in which a hybridization of a probe molecule to the amplicon is observed; and
   mark “0” for each channel in which no hybridization of a probe molecule to the amplicon is observed;
(j) removing the first group of probe molecules from the cells;
(k) repeating steps (g)-(j) for a number of iterations to exhaust the number of unique amplicon hybridization portions in the plurality of single-stranded DNA oligonucleotide probe molecules is exhausted, preferably 5-50 iterations, more preferably 8-12 iterations, more preferably 8 iterations,
   thereby generating a binary code for each amplicon location; and
(l) decoding the binary code for each amplicon location by mapping the binary code to a codebook which assigns the combined readout set of each barcoding oligonucleotide pair in the library to identification of a targeted molecule of the barcoding oligonucleotide pair, thereby identifying the polynucleotide molecule at the amplicon location, and
   thereby detecting a polynucleotide molecule from a library of targeted molecules.

In some embodiments, each generated binary code has a Hamming distance of at least 4 from any other binary code.

In some embodiments, each targeted molecule within the library of targeted molecules encodes a spacer sequence portion of a CRISPR guide RNA molecule.

In some embodiments, the cells are fixed cells, and preferably permeabilized cells, on a glass bottom well plate surface.

In some embodiments, the method is coupled with an additional readout, preferably with a multiplexed transcriptomic readout or with a cyclic immunofluorescence readout.

The invention also provides a composition comprising a transcript profiling oligonucleotide pair, the pair comprising
a) a single-stranded DNA padlock detection oligonucleotide molecule comprising
   i) a first primer detection oligonucleotide hybridization portion;
   ii) a padlock transcript-binding portion (H1) comprising a nucleotide sequence complementary to a first targeted nucleotide sequence (H1′) of a targeted polynucleotide molecule;
   iii) a first padlock readout portion (rs1);
   iv) a second padlock readout portion (rs2);
   v) a third padlock readout portion (rs3);
   vi) a second primer detection oligonucleotide hybridization portion; and
b) a single-stranded DNA primer detection oligonucleotide molecule comprising
   i) a primer transcript-binding portion (H2) comprising a nucleotide sequence complementary to a second targeted nucleotide sequence (H2′) of the targeted polynucleotide molecule;
   ii) a first padlock detection oligonucleotide hybridization portion comprising a nucleotide sequence complementary to the first primer detection oligonucleotide hybridization portion nucleotide sequence;
   iii) a splint hybridization primer readout portion (rs4); and
   iv) a second padlock detection oligonucleotide hybridization portion comprising a nucleotide sequence complementary to the second primer detection oligonucleotide hybridization portion nucleotide sequence.

In some embodiments, the splint hybridization primer readout portion (rs4) comprises a central splint hybridization portion and is flanked by additional nucleotides on each end, preferably wherein the central hybridization portion is about 20 nucleotides in length and is flanked by about 2 nucleotides on each end.

In some embodiments, the targeted polynucleotide molecule is an RNA transcript molecule encoded by a cell.

In some embodiments, (a)(i) to (a)(vi) of the padlock detection oligonucleotide molecule are arranged 5′ to 3′, respectively.

In some embodiments, the padlock detection oligonucleotide molecule comprises an intervening sequence portion between (a)(i) and (a)(ii), between (a)(ii) and (a)(iii), between (a)(iii) and (a)(iv), between (a)(iv) and (a)(v), and/or between (a)(v) and (a)(vi),
preferably wherein a first intervening sequence portion is between (a)(ii) and (a)(iii), and a second intervening sequence portion is between (a)(v) and (a)(vi), preferably wherein the first and second intervening sequence portions are each about 10-30 nucleotides in length,
and/or comprises an additional sequence portion upstream and/or downstream of any one of (a)(i) or (a)(vi).

In some embodiments, (b)(i) to (b)(iv) of the primer detection oligonucleotide molecule are arranged 5′ to 3′, respectively.

In some embodiments, the primer detection oligonucleotide molecule comprises an intervening sequence portion between (b)(i) and (b)(ii), between (b)(ii) and (b)(iii), and/or between (b)(iii) and (b)(iv), and/or comprises an additional sequence portion upstream and/or downstream of any one of (b)(i) or (b)(iv).

In some embodiments, H1, H1′, H2, H2′ are 20-50 nucleotides in length, preferably about 30 nucleotides in length,
rs1, rs2, rs3, and/or rs4 are 15-30 nucleotides in length, preferably about 20-25 nucleotides in length, more preferably about 20 nucleotides in length;
the padlock detection oligonucleotide molecule is 50-200, more preferably 50-150, more preferably 75-125 nucleotides in length, more preferably 100-110 nucleotides in length, more preferably about 106 nucleotides in length; and/or
the primer detection oligonucleotide molecule is 50-200, more preferably 50-150, more preferably 50-100 nucleotides in length.

In some embodiments, H1′ and H2′ are separated by no more than 10 nucleotides from each other on the targeted molecule, preferably wherein H1′ and H2′ are adjacent to each other.

In some embodiments, (a) the single-stranded DNA padlock detection oligonucleotide molecule and the (b) single-stranded DNA primer detection oligonucleotide molecule are not covalently bonded to each other.

In some embodiments, the composition further comprises a single-stranded DNA oligonucleotide splint molecule comprising a nucleotide sequence complementary to the rs4 nucleotide sequence.

In some embodiments, first splint molecule is 15-30 nucleotides in length, preferably about 20-25 nucleotides in length, more preferably 24 nucleotides in length, more preferably wherein the first splint molecule comprises a central splint hybridization portion consisting of a nucleotide sequence complementary to the first primer readout portion (rs4) flanked by additional nucleotides on each end, preferably wherein the central hybridization portion is about 20 nucleotides in length and is flanked by about 2 nucleotides on each end.

In some embodiments, the composition comprises a circularized amplicon template oligonucleotide molecule formed by ligation of one or more single-stranded DNA oligonucleotide splint molecules to the padlock detection oligonucleotide molecule.

In some embodiments, the composition further comprises one to four single-stranded DNA oligonucleotide probe molecules,
wherein a probe molecule of the one to four single-stranded DNA oligonucleotide probe molecules is conjugated to a detection molecule and comprises an amplicon hybridization portion;
   wherein the amplicon hybridization portion of a probe molecule of the one to four single-stranded DNA oligonucleotide probe molecules consists of
   i) a nucleotide sequence complementary to the first padlock readout portion (rs2) nucleotide sequence;
   ii) a nucleotide sequence complementary to the second padlock readout portion (rs1) nucleotide sequence;
   iii) the rs4 nucleotide sequence; or
   iv) the rs3 nucleotide sequence.

In some embodiments, the detection molecule is a fluorescent dye, Raman spectroscopy dye, a horse-radish peroxidase (HRP) enzyme, a depositing dye, or a mass spectroscopy imaging isotope.

In some embodiments, the detection molecule of at least one probe molecule has a different emission wavelength maxima than the detection molecule of another probe molecule of the composition, preferably such that the detection molecule of each probe molecule has a different emission wavelength maxima than the detection molecule of any other probe molecule of the composition.

In some embodiments, the composition comprises two to four probe molecules, wherein the amplicon hybridization portion of one probe molecule differs in sequence from the amplicon hybridization portion of another probe molecule, preferably wherein each probe molecule comprises an amplicon hybridization portion that differs in sequence from the amplicon hybridization portion of another probe molecule and is unique among the two to four probe molecules.

In some embodiments, the targeted polynucleotide molecule is an RNA molecule, preferably an mRNA molecule.

The invention also provides a kit comprising any of the compositions described above, optionally further comprising a DNA ligase and/or a DNA polymerase.

The invention also provides a transcript profiling library, the library comprising a plurality of transcript profiling oligonucleotide pairs described herein, wherein any targeted polynucleotide molecule is capable of being hybridized by 3-8 transcript profiling oligonucleotide pairs within the library.

The invention also provides a method of detecting a polynucleotide molecule in a cell, the method comprising
a) delivering to the cell the composition comprising a transcript profiling oligonucleotide pair described herein;
b) hybridizing the padlock detection molecule and the primer detection molecule to the polynucleotide molecule, and hybridizing the padlock detection molecule to the primer detection molecule;
c) delivering to the cell a single-stranded DNA oligonucleotide splint molecule comprising a nucleotide sequence complementary to the rs4 nucleotide sequence described herein;
d) hybridizing the first splint molecule to the primer detection molecule;
e) ligating the splint molecule to the hybridized padlock detection molecule to form a circularized amplicon template oligonucleotide molecule;
f) amplifying the circularized amplicon template oligonucleotide molecule by rolling circle amplification to form an amplicon molecule;
g) delivering to the cell one to four single-stranded DNA oligonucleotide probe molecules described herein,
   wherein each probe molecule of the one to four single-stranded DNA oligonucleotide probe molecules is conjugated to a detection molecule and comprises an amplicon hybridization portion;
   wherein the amplicon hybridization portion comprises a sequence selected from
      i) a nucleotide sequence complementary to the first padlock readout portion (rs2) nucleotide sequence;
      ii) a nucleotide sequence complementary to the second padlock readout portion (rs1) nucleotide sequence;
      iii) the rs4 nucleotide sequence; or
      iv) the rs3 nucleotide sequence; and
h) optically detecting the presence of the polynucleotide molecule in the cell upon hybridization of a probe molecule to the amplicon molecule, preferably optically detecting the presence of the polynucleotide molecule in the cell upon hybridization of each probe molecule delivered to the cell to the amplicon molecule.

In some embodiments, the one to four single-stranded DNA oligonucleotide probe molecules are delivered at about the same time or at different times.

In some embodiments, in step (g) a first probe molecule of the one to four probe molecules is delivered to the cell, step (h) is performed, and further comprising
i) removing the first probe molecule from the cell;
j) delivering to the cell a second single-stranded DNA oligonucleotide probe molecule described herein; and
k) optically detecting the presence of the polynucleotide molecule in the cell upon hybridization of the second probe molecule to the amplicon molecule.

In some embodiments, the method further comprises
l) removing the second probe molecule of from the cell;
m) delivering to the cell a third single-stranded DNA oligonucleotide probe molecule described herein; and
n) optically detecting the presence of the polynucleotide molecule in the cell upon hybridization of the third probe molecule to the amplicon molecule.

In some embodiments, the method further comprises
o) removing the third probe molecule from the cell;
p) delivering to the cell a fourth single-stranded DNA oligonucleotide probe molecule described herein; and
q) optically detecting the presence of the polynucleotide molecule in the cell upon hybridization of the second probe molecule to the amplicon molecule.

In some embodiments, the cells are fixed cells, and preferably permeabilized cells, and are preferably on a glass bottom well plate surface.

The invention also provides a cellular barcoding library, the library comprising a plurality of barcoding oligonucleotide pairs described herein,
wherein each barcoding oligonucleotide pair comprises a combined readout set of rs1, rs2, rs3 and rs4 sequences;
wherein each combined readout set is unique to its barcoding oligonucleotide pair within the library

In some embodiments, each first primer detection oligonucleotide hybridization portion is a universal sequence common to each padlock detection oligonucleotide molecule;
each second primer detection oligonucleotide hybridization portion is a universal sequence common to each padlock detection oligonucleotide molecule;
each first padlock detection oligonucleotide hybridization portion is a universal sequence common to each primer detection oligonucleotide molecule; and/or
each second padlock detection oligonucleotide hybridization portion is a universal sequence common to each primer detection oligonucleotide molecule.

In some embodiments, each targeted molecule in a library of targeted molecules comprises a unique targeted portion comprising a first targeted nucleotide sequence (H1′) and a second targeted nucleotide sequence (H2′),
(a) wherein the first targeted nucleotide sequence (H1′) is complementary to a padlock barcode portion (H1) of a padlock detection molecule of a barcoding oligonucleotide pair in the library; and
(b) wherein the second targeted nucleotide sequence (H2′) is complementary to the primer barcode portion (H2) of a primer detection molecule of the said barcoding oligonucleotide pair in (a);
   such that no two targeted molecules in the library of targeted molecules share the same unique targeted portion.

In some embodiments, each barcoding oligonucleotide pair is capable of hybridizing to only one targeted molecule in a library of targeted molecules while the padlock detection oligonucleotide molecule and the primer detection oligonucleotide molecule of the pair are simultaneously hybridized.

In some embodiments, the cellular barcoding library further comprises a plurality of single-stranded DNA oligonucleotide splint molecules, wherein each splint molecule comprises a nucleotide sequence complementary to a rs4 nucleotide sequence of a primer detection oligonucleotide molecule in the cellular barcoding library.

In some embodiments, the cellular barcoding library further comprises a plurality of DNA oligonucleotide probe molecules,
wherein each probe molecule is conjugated to a detection molecule and comprises an amplicon hybridization portion;
wherein the amplicon hybridization portion comprises
i) a nucleotide sequence complementary to a first padlock readout portion (rs2) nucleotide sequence of a padlock detection molecule of the cellular barcoding library;
ii) a nucleotide sequence complementary to a second padlock readout portion (rs1) nucleotide sequence of a padlock detection molecule of the cellular barcoding library;
iii) a rs4 nucleotide sequence of a primer detection molecule of the cellular barcoding library; or
iv) a rs3 nucleotide sequence of a padlock detection molecule of the cellular barcoding library.

In some embodiments, the number of unique amplicon hybridization portion sequences of the probe molecules in the plurality is at least equal to the number of unique sequences among the totality of combined readout sets of the oligonucleotide pairs within the library.

In some embodiments, the detection molecule is a fluorescent dye or Raman spectroscopy dye.

The invention also provides method of detecting a polynucleotide molecule from a library of targeted molecules in a cell, the method comprising
a) delivering a library of targeted molecules to a plurality of cells, preferably such that each cell in the plurality is delivered a targeted molecule;
b) delivering the cellular barcoding library described herein;
c) hybridizing the padlock detection molecule and the primer detection molecule of each barcoded oligonucleotide pair to a targeted polynucleotide molecule present in a cell, and hybridizing the padlock detection molecule to the primer detection molecule of each barcoded oligonucleotide pair;
d) delivering the plurality of splint molecules described herein;
e) ligating a splint molecule to a hybridized padlock detection molecule to form circularized amplicon template oligonucleotide molecules;
f) amplifying each circularized amplicon template oligonucleotide molecule by rolling circle amplification to form amplicon molecules;
g) delivering a first group of probe molecules from the plurality of single-stranded DNA oligonucleotide probe molecules described herein,
   wherein each probe molecule in the group comprises a unique amplicon hybridization portion which is different in sequence from the other probe molecules in the group;
   preferably wherein the number of probe molecules delivered in the group is equal to the number of probes that can be separately detected or spectrally distinguished in a readout cycle, preferably at most equal to a number of channels on a detection module, preferably a microscope to be used to optically view the cells, preferably wherein the number of probe molecules delivered in the group is 3 probe molecules;
   preferably wherein each probe molecule in the group is conjugated to a detection molecule with a different emission wavelength maxima than the other probe molecules in the group,
      more preferably wherein each probe molecule in the group is conjugated to a detection molecule having an emission wavelength maxima specific to only one channel of the microscope and no two detection molecules have an emission wavelength maxima in the same channel;
h) optically detecting the hybridization of the probe molecules from the first group to any amplicon molecule in any cell of the plurality of cells;
i) for each amplicon location,
   mark “1” for each channel in which a hybridization of a probe molecule to the amplicon is observed; and
   mark “0” for each channel in which no hybridization of a probe molecule to the amplicon is observed;
j) removing the first group of probe molecules from the cells;
k) repeating steps (g)-(j) for a number of iterations to exhaust the number of unique amplicon hybridization portions in the plurality of single-stranded DNA oligonucleotide probe molecules is exhausted, preferably 5-50 iterations, more preferably 8-12 iterations, more preferably 8 iterations, thereby generating a binary code for each amplicon location; and
l) decoding the binary code for each amplicon location by mapping the binary code to a codebook which assigns the combined readout set of each barcoding oligonucleotide pair in the library to identification of a targeted molecule of the barcoding oligonucleotide pair, thereby identifying the polynucleotide molecule at the amplicon location, and
thereby detecting a polynucleotide molecule from a library of targeted molecules.

In some embodiments, each generated binary code has a Hamming distance of at least 4 from any other binary code.

In some embodiments, the cells are fixed cells, and preferably permeabilized cells, on a glass bottom well plate surface.

In some embodiments, the method is coupled with an additional readout, preferably with a multiplexed transcriptomic readout or with a cyclic immunofluorescence readout.

The invention also provides a composition comprising a barcoding oligonucleotide set, the set comprising:
a) a single-stranded DNA left primer oligonucleotide molecule comprising
   i) a left universal bridge oligonucleotide hybridization portion;
   ii) a first left readout portion (rs1);
   iii) a second left readout portion (rs2);
   iv) a left padlock bottom oligonucleotide hybridization portion; and
   v) a left barcode portion (H1) comprising a nucleotide sequence complementary to a first targeted nucleotide sequence (H1′) of a targeted polynucleotide molecule; and
b) a single-stranded DNA padlock bottom oligonucleotide molecule comprising
   i) a right primer oligonucleotide hybridization portion;
   ii) a padlock bottom barcode portion (H2) comprising a nucleotide sequence complementary to a second targeted nucleotide sequence (H2′) of the targeted polynucleotide molecule; and
   iii) a left primer oligonucleotide hybridization portion comprising a nucleotide sequence complementary to the left padlock bottom oligonucleotide hybridization portion of the left primer oligonucleotide; and
c) a single-stranded DNA right primer oligonucleotide molecule comprising
   i) a right barcode portion (H3) comprising a nucleotide sequence complementary to a third targeted nucleotide sequence (H3′) of the targeted polynucleotide molecule nucleotide sequence;
   ii) a right padlock bottom oligonucleotide hybridization portion comprising a nucleotide sequence complementary to the right primer oligonucleotide hybridization portion of the bottom padlock molecule;
   iii) a first right primer readout portion (rs4);
   iv) a second right primer readout portion (rs3); and
   v) a right universal bridge oligonucleotide hybridization portion; and
d) a universal bridge oligonucleotide molecule comprising
   i) a left primer oligonucleotide hybridization portion comprising a nucleotide sequence complementary to the left universal bridge oligonucleotide hybridization portion nucleotide sequence;
   ii) an intervening bridge portion; and
   iii) a right primer oligonucleotide hybridization portion comprising a nucleotide sequence complementary to the right universal bridge oligonucleotide hybridization portion nucleotide sequence
      preferably wherein (d)(i) and (d)(iii) differ in melting temperature,
         preferably such that hybridization of (d)(i) to (a)(i) may occur separately from the hybridization of (d)(iii) to (c)(v) under a first hybridization condition; and
      may occur at the same as hybridization of (d)(iii) to (c)(v) under a second hybridization condition.

In some embodiments, the composition further comprises a single-stranded DNA oligonucleotide splint molecule comprising a nucleotide sequence complementary to the first left readout portion (rs1) nucleotide sequence.

In some embodiments, the composition further comprises a single-stranded DNA oligonucleotide splint molecule comprising a nucleotide sequence complementary to the second left readout portion (rs2) nucleotide sequence.

In some embodiments, the composition further comprises a single-stranded DNA oligonucleotide splint molecule comprising a nucleotide sequence complementary to the first right primer readout portion (rs4) nucleotide sequence.

In some embodiments, the composition further comprises a single-stranded DNA oligonucleotide splint molecule comprising a nucleotide sequence complementary to the second right primer readout portion (rs3) nucleotide sequence.

In some embodiments, the composition further comprises an intermediate padlock bottom-binding oligonucleotide molecule comprising
(i) a first intermediate primer readout portion (rs6);
(ii) a first intermediate primer readout portion (rs5); and
(iii) an amplicon padlock bottom barcode portion (aH2) comprising a nucleotide sequence complementary to the second targeted nucleotide sequence (H2′).

In some embodiments, the composition comprises at least one linker oligonucleotide which comprise a first readout sequence portion (rs5) and a second readout sequence portion (rs6).

In some embodiments, the composition further comprises a plurality of probe molecules which bind any one of the readout sequence portions.

In some embodiments, the composition is used in a genome-wide assay.

The invention also provides a kit comprising any of the compositions described above, optionally further comprising a DNA ligase and/or a DNA polymerase.

In embodiments, the methods described herein are used in an organoid system or organoid culture.

In embodiments, the methods are used in patient-derived organoids.

Notably, as described herein, pooled perturbations allow for cost efficient screening (e.g., less need for expensive Matrigel).

In embodiments the cells or organoids employed in the methods, or to which the methods are applied, are representative of Crohn's disease or the mammalian brain.

The methods employed herein can be used to analyze combinations of gene targets to revert phenotypes.

In embodiments, the methods herein allow for pooled perturbations in a cost efficient manner. For example, prior to this technology, to study 300 perturbations would need 300 cell lines while with the present invention only one cell line is needed.

A nonlimiting example of a padlock detection oligonucleotide and primer detection oligonucleotide combination is provided by SEQ ID NO: 6 (full length padlock sequence) and SEQ ID NO: 7 (full length primer sequence). A first and second readout probe for the padlock is provided by SEQ ID NO: 8 and SEQ ID NO: 9. A first and second readout probe for the primer is provided by SEQ ID NO: 10 and SEQ ID NO: 11. A first splint oligonucleotide sequence for this combination is provided by SEQ ID NO: 12. A second splint oligonucleotide sequence for this combination is provided by SEQ ID NO: 13. The padlock enc. sequence provided by SEQ ID NO: 14. The primer enc. sequence provided by SEQ ID NO: 15. The padlock enc. reverse complement sequence provided by SEQ ID NO: 16. The primer enc. reverse complement sequence provided by SEQ ID NO: 17.

Clause 1. A composition comprising a barcoding oligonucleotide pair, the pair comprising: (a) a single-stranded DNA padlock detection oligonucleotide molecule comprising (i) a first primer detection oligonucleotide hybridization portion; (ii) a padlock barcode portion (H1) comprising a nucleotide sequence complementary to a first targeted nucleotide sequence (H1′) of a targeted polynucleotide molecule; (iii) a first padlock readout portion (rs2); (iv) a second padlock readout portion (rs1); and (v) a second primer detection oligonucleotide hybridization portion; and (b) a single-stranded DNA primer detection oligonucleotide molecule comprising (i) a primer barcode portion (H2) comprising a nucleotide sequence complementary to a second targeted nucleotide sequence (H2′) of the targeted polynucleotide molecule; (ii) a first padlock detection oligonucleotide hybridization portion comprising a nucleotide sequence complementary to the first primer detection oligonucleotide hybridization portion nucleotide sequence; (iii) a first primer readout portion (rs4); (iv) a second primer readout portion (rs3); and (v) a second padlock detection oligonucleotide hybridization portion comprising a nucleotide sequence complementary to the second primer detection oligonucleotide hybridization portion nucleotide sequence.

Clause 2. The composition of clause 1, wherein (a)(i) to (a)(v) of the padlock detection oligonucleotide molecule are arranged 5′ to 3′, respectively.

Clause 3. The composition of any one of clauses 1 or 2, wherein the padlock detection oligonucleotide molecule comprises an intervening sequence portion between (a)(i) and (a)(ii), between (a)(ii) and (a)(iii), between (a)(iii) and (a)(iv), and/or between (a)(iv) and (a)(v), preferably wherein a first intervening sequence portion is between (a)(ii) and (a)(iii), and a second intervening sequence portion is between (a)(iv) and (a)(v), preferably wherein the first and second intervening sequence portions are each about 10-30 nucleotides in length, and/or comprises an additional sequence portion upstream and/or downstream of any one of (a)(i) or (a)(v).

Clause 4. The composition of any one of clauses 1-3, wherein (b)(i) to (b)(v) of the primer detection oligonucleotide molecule are arranged 5′ to 3′, respectively.

Clause 5. The composition of any one of clauses 1-4, wherein the primer detection oligonucleotide molecule comprises an intervening sequence portion between (b)(i) and (b)(ii), between (b)(ii) and (b)(iii), between (b)(iii) and (b)(iv), and/or between (b)(iv) and (b)(v) and/or comprises an additional sequence portion upstream and/or downstream of any one of (b)(i) or (b)(v).

Clause 6. The composition of any one of clauses 1-5, wherein H1, H1′, H2, H2′ are 20-50 nucleotides in length, preferably about 30 nucleotides in length rs1, rs2, rs3, and/or rs4 are 15-30 nucleotides in length, preferably about 20-25 nucleotides in length, more preferably about 20 nucleotides in length; the padlock detection oligonucleotide molecule is 50-200, more preferably 50-150, more preferably 75-125 nucleotides in length, more preferably 100-110 nucleotides in length, more preferably about 106 nucleotides in length; and/or the primer detection oligonucleotide molecule is 50-200, more preferably 50-150, more preferably 50-100 nucleotides in length.

Clause 7. The composition of any one of clauses 1-6, wherein H1′ and H2′ are separated by no more than 10 nucleotides from each other on the targeted molecule, preferably wherein H1′ and H2′ are adjacent to each other.

Clause 8. The composition of any one of clauses 1-7, wherein (a) the single-stranded DNA padlock detection oligonucleotide molecule and the (b) single-stranded DNA primer detection oligonucleotide molecule are not covalently bonded to each other.

Clause 9. The composition of any one of clauses 1-8, further comprising a first single-stranded DNA oligonucleotide splint molecule comprising a nucleotide sequence complementary to the first primer readout portion (rs4) nucleotide sequence.

Clause 10. The composition of clause 9, wherein the first splint molecule is 15-30 nucleotides in length, preferably about 20-25 nucleotides in length, more preferably 24 nucleotides in length, more preferably wherein the first splint molecule comprises a central splint hybridization portion consisting of a nucleotide sequence complementary to the first primer readout portion (rs4) flanked by additional nucleotides on each end, preferably wherein the central hybridization portion is about 20 nucleotides in length and is flanked by about 2 nucleotides on each end.

Clause 11. The composition of any one of clauses 1-10, further comprising a second single-stranded DNA oligonucleotide splint molecule comprising a nucleotide sequence complementary to the first primer readout portion (rs3) nucleotide sequence.

Clause 12. The composition of clause 11, wherein the second splint molecule is 15-30 nucleotides in length, preferably about 20-25 nucleotides in length, more preferably 24 nucleotides in length, more preferably wherein the second splint molecule comprises a central splint hybridization portion consisting of a nucleotide sequence complementary to the first primer readout portion (rs3) flanked by additional nucleotides on each end, preferably wherein the central hybridization portion is about 20 nucleotides in length and is flanked by about 2 nucleotides on each end.

Clause 13. The composition of any one of clauses 9-12, further comprising a circularized amplicon template oligonucleotide molecule formed by ligation of one, two, or more single-stranded DNA oligonucleotide splint molecules to the padlock detection oligonucleotide molecule.

Clause 14. The composition of any one of clauses 1-13, comprising one to four single-stranded DNA oligonucleotide probe molecules, wherein a probe molecule of the one to four single-stranded DNA oligonucleotide probe molecules is conjugated to a detection molecule and comprises an amplicon hybridization portion; wherein the amplicon hybridization portion of a probe molecule of the one to four single-stranded DNA oligonucleotide probe molecules consists of (i) a nucleotide sequence complementary to the first padlock readout portion (rs2) nucleotide sequence; (ii) a nucleotide sequence complementary to the second padlock readout portion (rs1) nucleotide sequence; (iii) the first primer readout portion (rs4) nucleotide sequence; or (iv) the second primer readout portion (rs3) nucleotide sequence.

Clause 15. The composition of clause 14, wherein the detection molecule is a fluorescent dye, Raman spectroscopy dye, a horse-radish peroxidase (HRP) enzyme, a depositing dye, or a mass spectroscopy imaging isotope.

Clause 16. The composition of any one of clauses 14 or 15, wherein the detection molecule of at least one probe molecule has a different emission wavelength maxima than the detection molecule of another probe molecule of the composition, preferably such that the detection molecule of each probe molecule has a different emission wavelength maxima than the detection molecule of any other probe molecule of the composition.

Clause 17. The composition of any one of clauses 14-16, comprising two to four probe molecules, wherein the amplicon hybridization portion of one probe molecule differs in sequence from the amplicon hybridization portion of another probe molecule, preferably wherein each probe molecule comprises an amplicon hybridization portion that differs in sequence from the amplicon hybridization portion of another probe molecule and is unique among the two to four probe molecules.

Clause 18. The composition of any one of clauses 1-17, wherein the targeted polynucleotide molecule is an RNA molecule.

Clause 19. The composition of any one of clauses 1-18, wherein the targeted polynucleotide molecule encodes a spacer sequence portion of a CRISPR guide RNA molecule.

Clause 20. The composition of any one of clauses 1-19, further comprising a DNA polynucleotide encoding the targeted polynucleotide molecule.

Clause 21. A kit comprising the composition of any one of clauses 1-20, optionally further comprising a DNA ligase and/or a DNA polymerase.

Clause 22. A method of detecting a polynucleotide molecule in a cell, the method comprising: (a) delivering to the cell the composition according to any one of clauses 1-8; (b) hybridizing the padlock detection molecule and the primer detection molecule to the polynucleotide molecule, and hybridizing the padlock detection molecule to the primer detection molecule; (c) delivering to the cell (i) a first single-stranded DNA oligonucleotide splint molecule comprising a nucleotide sequence complementary to the first primer readout portion (rs4) nucleotide sequence according to any one of clauses 9 or 10; and (ii) a second single-stranded DNA oligonucleotide splint molecule comprising a nucleotide sequence complementary to the first primer readout portion (rs3) nucleotide sequence according to any one of clauses 11 or 12; (d) hybridizing the first splint molecule to the primer detection molecule and hybridizing the second splint molecule to the primer detection molecule; (e) ligating the first and second splint molecules to the hybridized padlock detection molecule to form a circularized amplicon template oligonucleotide molecule; (f) amplifying the circularized amplicon template oligonucleotide molecule by rolling circle amplification to form an amplicon molecule; (g) delivering to the cell one to four single-stranded DNA oligonucleotide probe molecules according to any one of clauses 14-17, wherein each probe molecule of the one to four single-stranded DNA oligonucleotide probe molecules is conjugated to a detection molecule and comprises an amplicon hybridization portion; wherein the amplicon hybridization portion comprises a sequence selected from (i) a nucleotide sequence complementary to the first padlock readout portion (rs2) nucleotide sequence; (ii) a nucleotide sequence complementary to the second padlock readout portion (rs1) nucleotide sequence; (iii) the first primer readout portion (rs4) nucleotide sequence; or (iv) the second primer readout portion (rs3) nucleotide sequence; and (h) optically detecting the presence of the polynucleotide molecule in the cell upon hybridization of a probe molecule to the amplicon molecule, preferably optically detecting the presence of the polynucleotide molecule in the cell upon hybridization of each probe molecule delivered to the cell to the amplicon molecule.

Clause 23. The method of claim 22, wherein the one to four single-stranded DNA oligonucleotide probe molecules are delivered at about the same time or at different times.

Clause 24. The method of clause 22 or 23, wherein in step (g) a first probe molecule of the one to four probe molecules is delivered to the cell, step (h) is performed, and further comprising (i) removing the first probe molecule from the cell; (j) delivering to the cell a second single-stranded DNA oligonucleotide probe molecule according to any one of clauses 14-17; and (k) optically detecting the presence of the polynucleotide molecule in the cell upon hybridization of the second probe molecule to the amplicon molecule.

Clause 25. The method of clause 24, further comprising (1) removing the second probe molecule of from the cell; (m) delivering to the cell a third single-stranded DNA oligonucleotide probe molecule according to any one of clauses 14-17; and (n) optically detecting the presence of the polynucleotide molecule in the cell upon hybridization of the third probe molecule to the amplicon molecule.

Clause 26. The method of clause 25, further comprising (o) removing the third probe molecule from the cell; (p) delivering to the cell a fourth single-stranded DNA oligonucleotide probe molecule according to any one of clauses 14-17; and (q) optically detecting the presence of the polynucleotide molecule in the cell upon hybridization of the second probe molecule to the amplicon molecule.

Clause 27. The method of any one clauses 22-26, wherein the cells are fixed cells, and preferably permeabilized cells, and are preferably on a glass bottom well plate surface.

Clause 28. A cellular barcoding library, the library comprising a plurality of barcoding oligonucleotide pairs according to any one of clauses 1-8, wherein each barcoding oligonucleotide pair comprises a combined readout set of rs1, rs2, rs3 and rs4 sequences; wherein each combined readout set is unique to its barcoding oligonucleotide pair within the library.

Clause 29. The cellular barcoding library of clause 28, wherein each first primer detection oligonucleotide hybridization portion is a universal sequence common to each padlock detection oligonucleotide molecule; each second primer detection oligonucleotide hybridization portion is a universal sequence common to each padlock detection oligonucleotide molecule; each first padlock detection oligonucleotide hybridization portion is a universal sequence common to each primer detection oligonucleotide molecule; and/or each second padlock detection oligonucleotide hybridization portion is a universal sequence common to each primer detection oligonucleotide molecule.

Clause 30. The cellular barcoding library of any one of clauses 28 or 29, wherein each targeted molecule in a library of targeted molecules comprises a unique targeted portion comprising a first targeted nucleotide sequence (H1′) and a second targeted nucleotide sequence (H2′), (a) wherein the first targeted nucleotide sequence (H1′) is complementary to a padlock barcode portion (H1) of a padlock detection molecule of a barcoding oligonucleotide pair in the library; and (b) wherein the second targeted nucleotide sequence (H2′) is complementary to the primer barcode portion (H2) of a primer detection molecule of the said barcoding oligonucleotide pair in (a); such that no two targeted molecules in the library of targeted molecules share the same unique targeted portion.

Clause 31. The cellular barcoding library of any one of clauses 28-30, wherein each barcoding oligonucleotide pair is capable of hybridizing to only one targeted molecule in a library of targeted molecules while the padlock detection oligonucleotide molecule and the primer detection oligonucleotide molecule of the pair are simultaneously hybridized.

Clause 32. The cellular barcoding library of any one of clauses 28-31, further comprising a plurality of single-stranded DNA oligonucleotide splint molecules, wherein each splint molecule comprises a nucleotide sequence complementary to a first primer readout portion (rs4) nucleotide sequence of a primer detection oligonucleotide molecule in the cellular barcoding library.

Clause 33. The cellular barcoding library of any one of clauses 28-32, further comprising a second plurality of single-stranded DNA oligonucleotide splint molecules, wherein each splint molecule in the second plurality comprises a nucleotide sequence complementary to a second primer readout portion (rs3) nucleotide sequence of a primer detection oligonucleotide molecule in the cellular barcoding library.

Clause 34. The cellular barcoding library of any one of clauses 28-33, further comprising a plurality of DNA oligonucleotide probe molecules, wherein each probe molecule is conjugated to a detection molecule and comprises an amplicon hybridization portion; wherein the amplicon hybridization portion comprises (i) a nucleotide sequence complementary to a first padlock readout portion (rs2) nucleotide sequence of a padlock detection molecule of the cellular barcoding library; (ii) a nucleotide sequence complementary to a second padlock readout portion (rs1) nucleotide sequence of a padlock detection molecule of the cellular barcoding library; (iii) a first primer readout portion (rs4) nucleotide sequence of a primer detection molecule of the cellular barcoding library; or (iv) a second primer readout portion (rs3) nucleotide sequence of a primer detection molecule of the cellular barcoding library.

Clause 35. The cellular barcoding library comprising a plurality of probe molecules of clause 34, wherein the number of unique amplicon hybridization portion sequences of the probe molecules in the plurality is at least equal to the number of unique sequences among the totality of combined readout sets of the oligonucleotide pairs within the library.

Clause 36. The cellular barcoding library comprising a plurality of probe molecules of any one of clauses 34 or 35, wherein the detection molecule is a fluorescent dye or Raman spectroscopy dye.

Clause 37. A method of detecting a polynucleotide molecule from a library of targeted molecules in a cell, the method comprising (a) delivering a library of targeted molecules to a plurality of cells, preferably such that each cell in the plurality is delivered a targeted molecule; (b) delivering the cellular barcoding library of any one of clauses 28-31; (c) hybridizing the padlock detection molecule and the primer detection molecule of each barcoded oligonucleotide pair to a targeted polynucleotide molecule present in a cell, and hybridizing the padlock detection molecule to the primer detection molecule of each barcoded oligonucleotide pair; (d) delivering the plurality of splint molecules of clause 32 and the second plurality of splint molecules of clause 33; (e) ligating a first and a second splint molecule to a hybridized padlock detection molecule to form circularized amplicon template oligonucleotide molecules; (f) amplifying each circularized amplicon template oligonucleotide molecule by rolling circle amplification to form amplicon molecules; (g) delivering a first group of probe molecules from the plurality of single-stranded DNA oligonucleotide probe molecules according to any one of clauses 34-36, wherein each probe molecule in the group comprises a unique amplicon hybridization portion which is different in sequence from the other probe molecules in the group; preferably wherein the number of probe molecules delivered in the group is equal to the number of probes that can be separately detected or spectrally distinguished in a readout cycle, preferably at most equal to a number of channels on a detection module, preferably a microscope to be used to optically view the cells, preferably wherein the number of probe molecules delivered in the group is 3 probe molecules; preferably wherein each probe molecule in the group is conjugated to a detection molecule with a different emission wavelength maxima than the other probe molecules in the group, more preferably wherein each probe molecule in the group is conjugated to a detection molecule having an emission wavelength maxima specific to only one channel of the microscope and no two detection molecules have an emission wavelength maxima in the same channel; (h) optically detecting the hybridization of the probe molecules from the first group to any amplicon molecule in any cell of the plurality of cells; (i) for each amplicon location, mark “1” for each channel in which a hybridization of a probe molecule to the amplicon is observed; and mark “0” for each channel in which no hybridization of a probe molecule to the amplicon is observed; (j) removing the first group of probe molecules from the cells; (k) repeating steps (g)-(j) for a number of iterations to exhaust the number of unique amplicon hybridization portions in the plurality of single-stranded DNA oligonucleotide probe molecules is exhausted, preferably 5-50 iterations, more preferably 8-12 iterations, more preferably 8 iterations, thereby generating a binary code for each amplicon location; and (1) decoding the binary code for each amplicon location by mapping the binary code to a codebook which assigns the combined readout set of each barcoding oligonucleotide pair in the library to identification of a targeted molecule of the barcoding oligonucleotide pair, thereby identifying the polynucleotide molecule at the amplicon location, and thereby detecting a polynucleotide molecule from a library of targeted molecules.

Clause 37. The method of clause 37, wherein each generated binary code has a Hamming distance of at least 4 from any other binary code.

Clause 38. The method of any one of clauses 37 or 38, wherein each targeted molecule within the library of targeted molecules encodes a spacer sequence portion of a CRISPR guide RNA molecule.

Clause 39. The method of any one clauses 37-39, wherein the cells are fixed cells, and preferably permeabilized cells, on a glass bottom well plate surface.

Clause 40. The method of any one of clauses 37-40, wherein the method is coupled with an additional readout, preferably with a multiplexed transcriptomic readout or with a cyclic immunofluorescence readout.

Clause 41. A composition comprising a transcript profiling oligonucleotide pair, the pair comprising: (a) a single-stranded DNA padlock detection oligonucleotide molecule comprising (i) a first primer detection oligonucleotide hybridization portion; (ii) a padlock transcript-binding portion (H1) comprising a nucleotide sequence complementary to a first targeted nucleotide sequence (H1′) of a targeted polynucleotide molecule; (iii) a first padlock readout portion (rs1); (iv) a second padlock readout portion (rs2); (v) a third padlock readout portion (rs3); (vi) a second primer detection oligonucleotide hybridization portion; and (b) a single-stranded DNA primer detection oligonucleotide molecule comprising (i) a primer transcript-binding portion (H2) comprising a nucleotide sequence complementary to a second targeted nucleotide sequence (H2′) of the targeted polynucleotide molecule; (ii) a first padlock detection oligonucleotide hybridization portion comprising a nucleotide sequence complementary to the first primer detection oligonucleotide hybridization portion nucleotide sequence; (iii) a splint hybridization primer readout portion (rs4); and (iv) a second padlock detection oligonucleotide hybridization portion comprising a nucleotide sequence complementary to the second primer detection oligonucleotide hybridization portion nucleotide sequence.

Clause 43. The composition of clause 42, wherein the splint hybridization primer readout portion (rs4) comprises a central splint hybridization portion and is flanked by additional nucleotides on each end, preferably wherein the central hybridization portion is about 20 nucleotides in length and is flanked by about 2 nucleotides on each end.

Clause 44. The composition of any one of clauses 42 or 43, wherein the targeted polynucleotide molecule is an RNA transcript molecule encoded by a cell.

Clause 45. The composition of any one of clauses 42-44, wherein (a)(i) to (a)(vi) of the padlock detection oligonucleotide molecule are arranged 5′ to 3′, respectively.

Clause 46. The composition of any one of clauses 42-45, wherein the padlock detection oligonucleotide molecule comprises an intervening sequence portion between (a)(i) and (a)(ii), between (a)(ii) and (a)(iii), between (a)(iii) and (a)(iv), between (a)(iv) and (a)(v), and/or between (a)(v) and (a)(vi), preferably wherein a first intervening sequence portion is between (a)(ii) and (a)(iii), and a second intervening sequence portion is between (a)(v) and (a)(vi), preferably wherein the first and second intervening sequence portions are each about 10-30 nucleotides in length, and/or comprises an additional sequence portion upstream and/or downstream of any one of (a)(i) or (a)(vi).

Clause 47. The composition of any one of clauses 42-46, wherein (b)(i) to (b)(iv) of the primer detection oligonucleotide molecule are arranged 5′ to 3′, respectively.

Clause 48. The composition of any one of clauses 42-47, wherein the primer detection oligonucleotide molecule comprises an intervening sequence portion between (b)(i) and (b)(ii), between (b)(ii) and (b)(iii), and/or between (b)(iii) and (b)(iv), and/or comprises an additional sequence portion upstream and/or downstream of any one of (b)(i) or (b)(iv).

Clause 49. The composition of any one of clauses 42-48, wherein H1, H1′, H2, H2′ are 20-50 nucleotides in length, preferably about 30 nucleotides in length rs1, rs2, rs3, and/or rs4 are 15-30 nucleotides in length, preferably about 20-25 nucleotides in length, more preferably about 20 nucleotides in length; the padlock detection oligonucleotide molecule is 50-200, more preferably 50-150, more preferably 75-125 nucleotides in length, more preferably 100-110 nucleotides in length, more preferably about 106 nucleotides in length; and/or the primer detection oligonucleotide molecule is 50-200, more preferably 50-150, more preferably 50-100 nucleotides in length.

Clause 50. The composition of any one of clauses 42-49, wherein H1′ and H2′ are separated by no more than 10 nucleotides from each other on the targeted molecule, preferably wherein H1′ and H2′ are adjacent to each other.

Clause 51. The composition of any one of clauses 42-50, wherein (a) the single-stranded DNA padlock detection oligonucleotide molecule and the (b) single-stranded DNA primer detection oligonucleotide molecule are not covalently bonded to each other.

Clause 52. The composition of any one of clauses 42-51, further comprising a single-stranded DNA oligonucleotide splint molecule comprising a nucleotide sequence complementary to the rs4 nucleotide sequence.

Clause 53. The composition of clause 52, wherein the first splint molecule is 15-30 nucleotides in length, preferably about 20-25 nucleotides in length, more preferably 24 nucleotides in length, more preferably wherein the first splint molecule comprises a central splint hybridization portion consisting of a nucleotide sequence complementary to the first primer readout portion (rs4) flanked by additional nucleotides on each end, preferably wherein the central hybridization portion is about 20 nucleotides in length and is flanked by about 2 nucleotides on each end.

Clause 54. The composition of clause 52 or 53, comprising a circularized amplicon template oligonucleotide molecule formed by ligation of one or more single-stranded DNA oligonucleotide splint molecules to the padlock detection oligonucleotide molecule.

Clause 55. The composition of any one of clauses 42-54, further comprising one to four single-stranded DNA oligonucleotide probe molecules, wherein a probe molecule of the one to four single-stranded DNA oligonucleotide probe molecules is conjugated to a detection molecule and comprises an amplicon hybridization portion; wherein the amplicon hybridization portion of a probe molecule of the one to four single-stranded DNA oligonucleotide probe molecules consists of (i) a nucleotide sequence complementary to the first padlock readout portion (rs2) nucleotide sequence; (ii) a nucleotide sequence complementary to the second padlock readout portion (rs1) nucleotide sequence; (iii) the rs4 nucleotide sequence; or (iv) the rs3 nucleotide sequence.

Clause 56. The composition of clause 55, wherein the detection molecule is a fluorescent dye, Raman spectroscopy dye, a horse-radish peroxidase (HRP) enzyme, a depositing dye, or a mass spectroscopy imaging isotope.

Clause 57. The composition of any one of clauses 55 or 56, wherein the detection molecule of at least one probe molecule has a different emission wavelength maxima than the detection molecule of another probe molecule of the composition, preferably such that the detection molecule of each probe molecule has a different emission wavelength maxima than the detection molecule of any other probe molecule of the composition.

Clause 58. The composition of any one of clauses 55-57, comprising two to four probe molecules, wherein the amplicon hybridization portion of one probe molecule differs in sequence from the amplicon hybridization portion of another probe molecule, preferably wherein each probe molecule comprises an amplicon hybridization portion that differs in sequence from the amplicon hybridization portion of another probe molecule and is unique among the two to four probe molecules.

Clause 59. The composition of any one of clauses 42-58, wherein the targeted polynucleotide molecule is an RNA molecule, preferably an mRNA molecule.

Clause 60. A kit comprising the composition of any one of clauses 42-59, optionally further comprising a DNA ligase and/or a DNA polymerase.

Clause 61. A transcript profiling library, the library comprising a plurality of transcript profiling oligonucleotide pairs according to any one of clauses 42-59, wherein any targeted polynucleotide molecule is capable of being hybridized by 3-8 transcript profiling oligonucleotide pairs within the library.

Clause 62. A method of detecting a polynucleotide molecule in a cell, the method comprising: (a) delivering to the cell the composition according to any one of clauses 42-51; (b) hybridizing the padlock detection molecule and the primer detection molecule to the polynucleotide molecule, and hybridizing the padlock detection molecule to the primer detection molecule; (c) delivering to the cell a single-stranded DNA oligonucleotide splint molecule comprising a nucleotide sequence complementary to the rs4 nucleotide sequence according to clause 52 or 53; (d) hybridizing the first splint molecule to the primer detection molecule; (e) ligating the splint molecule to the hybridized padlock detection molecule to form a circularized amplicon template oligonucleotide molecule; (f) amplifying the circularized amplicon template oligonucleotide molecule by rolling circle amplification to form an amplicon molecule; (g) delivering to the cell one to four single-stranded DNA oligonucleotide probe molecules according to any one of clauses 55-58, wherein each probe molecule of the one to four single-stranded DNA oligonucleotide probe molecules is conjugated to a detection molecule and comprises an amplicon hybridization portion; wherein the amplicon hybridization portion comprises a sequence selected from (i) a nucleotide sequence complementary to the first padlock readout portion (rs2) nucleotide sequence; (ii) a nucleotide sequence complementary to the second padlock readout portion (rs1) nucleotide sequence; (iii) the rs4 nucleotide sequence; or (iv) the rs3 nucleotide sequence; and (h) optically detecting the presence of the polynucleotide molecule in the cell upon hybridization of a probe molecule to the amplicon molecule, preferably optically detecting the presence of the polynucleotide molecule in the cell upon hybridization of each probe molecule delivered to the cell to the amplicon molecule.

Clause 63. The method of claim 62, wherein the one to four single-stranded DNA oligonucleotide probe molecules are delivered at about the same time or at different times.

Clause 64. The method of clause 62 or 63, wherein in step (g) a first probe molecule of the one to four probe molecules is delivered to the cell, step (h) is performed, and further comprising (i) removing the first probe molecule from the cell; (j) delivering to the cell a second single-stranded DNA oligonucleotide probe molecule according to any one of clauses 55-58; and (k) optically detecting the presence of the polynucleotide molecule in the cell upon hybridization of the second probe molecule to the amplicon molecule.

Clause 65. The method of clause 64, further comprising (1) removing the second probe molecule of from the cell; (m) delivering to the cell a third single-stranded DNA oligonucleotide probe molecule according to clause 52 or 53; and (n) optically detecting the presence of the polynucleotide molecule in the cell upon hybridization of the third probe molecule to the amplicon molecule.

Clause 66. The method of clause 65, further comprising (o) removing the third probe molecule from the cell; (p) delivering to the cell a fourth single-stranded DNA oligonucleotide probe molecule according to clause 52 or 53; and (q) optically detecting the presence of the polynucleotide molecule in the cell upon hybridization of the second probe molecule to the amplicon molecule.

Clause 67. The method of any one clauses 62-66, wherein the cells are fixed cells, and preferably permeabilized cells, and are preferably on a glass bottom well plate surface.

Clause 68. A cellular barcoding library, the library comprising a plurality of barcoding oligonucleotide pairs according to any one of clauses 42-51, wherein each barcoding oligonucleotide pair comprises a combined readout set of rs1, rs2, rs3 and rs4 sequences; wherein each combined readout set is unique to its barcoding oligonucleotide pair within the library.

Clause 69. The cellular barcoding library of clause 68, wherein each first primer detection oligonucleotide hybridization portion is a universal sequence common to each padlock detection oligonucleotide molecule; each second primer detection oligonucleotide hybridization portion is a universal sequence common to each padlock detection oligonucleotide molecule; each first padlock detection oligonucleotide hybridization portion is a universal sequence common to each primer detection oligonucleotide molecule; and/or each second padlock detection oligonucleotide hybridization portion is a universal sequence common to each primer detection oligonucleotide molecule.

Clause 70. The cellular barcoding library of any one of clauses 68 or 69, wherein each targeted molecule in a library of targeted molecules comprises a unique targeted portion comprising a first targeted nucleotide sequence (H1′) and a second targeted nucleotide sequence (H2′), (a) wherein the first targeted nucleotide sequence (H1′) is complementary to a padlock barcode portion (H1) of a padlock detection molecule of a barcoding oligonucleotide pair in the library; and (b) wherein the second targeted nucleotide sequence (H2′) is complementary to the primer barcode portion (H2) of a primer detection molecule of the said barcoding oligonucleotide pair in (a); such that no two targeted molecules in the library of targeted molecules share the same unique targeted portion.

Clause 71. The cellular barcoding library of any one of clauses 68-70, wherein each barcoding oligonucleotide pair is capable of hybridizing to only one targeted molecule in a library of targeted molecules while the padlock detection oligonucleotide molecule and the primer detection oligonucleotide molecule of the pair are simultaneously hybridized.

Clause 72. The cellular barcoding library of any one of clauses 68-71, further comprising a plurality of single-stranded DNA oligonucleotide splint molecules, wherein each splint molecule comprises a nucleotide sequence complementary to a rs4 nucleotide sequence of a primer detection oligonucleotide molecule in the cellular barcoding library.

Clause 73. The cellular barcoding library of any one of clauses 68-72, further comprising a plurality of DNA oligonucleotide probe molecules, wherein each probe molecule is conjugated to a detection molecule and comprises an amplicon hybridization portion; wherein the amplicon hybridization portion comprises (i) a nucleotide sequence complementary to a first padlock readout portion (rs2) nucleotide sequence of a padlock detection molecule of the cellular barcoding library; (ii) a nucleotide sequence complementary to a second padlock readout portion (rs1) nucleotide sequence of a padlock detection molecule of the cellular barcoding library; (iii) a rs4 nucleotide sequence of a primer detection molecule of the cellular barcoding library; or (iv) a rs3 nucleotide sequence of a padlock detection molecule of the cellular barcoding library.

Clause 74. The cellular barcoding library comprising a plurality of probe molecules of clause 73, wherein the number of unique amplicon hybridization portion sequences of the probe molecules in the plurality is at least equal to the number of unique sequences among the totality of combined readout sets of the oligonucleotide pairs within the library.

Clause 75. The cellular barcoding library comprising a plurality of probe molecules of any one of clauses 73 or 74, wherein the detection molecule is a fluorescent dye or Raman spectroscopy dye.

Clause 76. A method of detecting a polynucleotide molecule from a library of targeted molecules in a cell, the method comprising (a) delivering a library of targeted molecules to a plurality of cells, preferably such that each cell in the plurality is delivered a targeted molecule; (b) delivering the cellular barcoding library of any one of clauses 68-71; (c) hybridizing the padlock detection molecule and the primer detection molecule of each barcoded oligonucleotide pair to a targeted polynucleotide molecule present in a cell, and hybridizing the padlock detection molecule to the primer detection molecule of each barcoded oligonucleotide pair; (d) delivering the plurality of splint molecules of clause 72; (e) ligating a splint molecule to a hybridized padlock detection molecule to form circularized amplicon template oligonucleotide molecules; (f) amplifying each circularized amplicon template oligonucleotide molecule by rolling circle amplification to form amplicon molecules; (g) delivering a first group of probe molecules from the plurality of single-stranded DNA oligonucleotide probe molecules according to any one of clauses 73-75, wherein each probe molecule in the group comprises a unique amplicon hybridization portion which is different in sequence from the other probe molecules in the group; preferably wherein the number of probe molecules delivered in the group is equal to the number of probes that can be separately detected or spectrally distinguished in a readout cycle, preferably at most equal to a number of channels on a detection module, preferably a microscope to be used to optically view the cells, preferably wherein the number of probe molecules delivered in the group is 3 probe molecules; preferably wherein each probe molecule in the group is conjugated to a detection molecule with a different emission wavelength maxima than the other probe molecules in the group, more preferably wherein each probe molecule in the group is conjugated to a detection molecule having an emission wavelength maxima specific to only one channel of the microscope and no two detection molecules have an emission wavelength maxima in the same channel; (h) optically detecting the hybridization of the probe molecules from the first group to any amplicon molecule in any cell of the plurality of cells; (i) for each amplicon location, mark “1” for each channel in which a hybridization of a probe molecule to the amplicon is observed; and mark “0” for each channel in which no hybridization of a probe molecule to the amplicon is observed; (j) removing the first group of probe molecules from the cells; (k) repeating steps (g)-(j) for a number of iterations to exhaust the number of unique amplicon hybridization portions in the plurality of single-stranded DNA oligonucleotide probe molecules is exhausted, preferably 5-50 iterations, more preferably 8-12 iterations, more preferably 8 iterations, thereby generating a binary code for each amplicon location; and (1) decoding the binary code for each amplicon location by mapping the binary code to a codebook which assigns the combined readout set of each barcoding oligonucleotide pair in the library to identification of a targeted molecule of the barcoding oligonucleotide pair, thereby identifying the polynucleotide molecule at the amplicon location, and thereby detecting a polynucleotide molecule from a library of targeted molecules.

Clause 77. The method of clause 76, wherein each generated binary code has a Hamming distance of at least 4 from any other binary code.

Clause 78. The method of clause 76 or 77, wherein the cells are fixed cells, and preferably permeabilized cells, on a glass bottom well plate surface.

Clause 79. The method of any one of clauses 76-78, wherein the method is coupled with an additional readout, preferably with a multiplexed transcriptomic readout or with a cyclic immunofluorescence readout.

Clause 80. A composition comprising a barcoding oligonucleotide set, the set comprising: (a) a single-stranded DNA left primer oligonucleotide molecule comprising (i) a left universal bridge oligonucleotide hybridization portion; (ii) a first left readout portion (rs1); (iii) a second left readout portion (rs2); (iv) a left padlock bottom oligonucleotide hybridization portion; and (v) a left barcode portion (H1) comprising a nucleotide sequence complementary to a first targeted nucleotide sequence (H1′) of a targeted polynucleotide molecule; and (b) a single-stranded DNA padlock bottom oligonucleotide molecule comprising (i) a right primer oligonucleotide hybridization portion; (ii) a padlock bottom barcode portion (H2) comprising a nucleotide sequence complementary to a second targeted nucleotide sequence (H2′) of the targeted polynucleotide molecule; and (iii) a left primer oligonucleotide hybridization portion comprising a nucleotide sequence complementary to the left padlock bottom oligonucleotide hybridization portion of the left primer oligonucleotide; and (c) a single-stranded DNA right primer oligonucleotide molecule comprising (i) a right barcode portion (H3) comprising a nucleotide sequence complementary to a third targeted nucleotide sequence (H3′) of the targeted polynucleotide molecule nucleotide sequence; (ii) a right padlock bottom oligonucleotide hybridization portion comprising a nucleotide sequence complementary to the right primer oligonucleotide hybridization portion of the bottom padlock molecule; (iii) a first right primer readout portion (rs4); (iv) a second right primer readout portion (rs3); and (v) a right universal bridge oligonucleotide hybridization portion; and (d) a universal bridge oligonucleotide molecule comprising (i) a left primer oligonucleotide hybridization portion comprising a nucleotide sequence complementary to the left universal bridge oligonucleotide hybridization portion nucleotide sequence; (ii) an intervening bridge portion; and (iii) a right primer oligonucleotide hybridization portion comprising a nucleotide sequence complementary to the right universal bridge oligonucleotide hybridization portion nucleotide sequence preferably wherein (d)(i) and (d)(iii) differ in melting temperature, preferably such that hybridization of (d)(i) to (a)(i) may occur separately from the hybridization of (d)(iii) to (c)(v) under a first hybridization condition; and may occur at the same as hybridization of (d)(iii) to (c)(v) under a second hybridization condition.

Clause 81. The composition of clause 80, further comprising, a single-stranded DNA oligonucleotide splint molecule comprising a nucleotide sequence complementary to the first left readout portion (rs1) nucleotide sequence.

Clause 82. The composition of any one of clauses 80 or 81, further comprising, a single-stranded DNA oligonucleotide splint molecule comprising a nucleotide sequence complementary to the second left readout portion (rs2) nucleotide sequence.

Clause 83. The composition of any one of clauses 80-82, further comprising, a single-stranded DNA oligonucleotide splint molecule comprising a nucleotide sequence complementary to the first right primer readout portion (rs4) nucleotide sequence.

Clause 84. The composition of any one of clauses 80-83, further comprising, a single-stranded DNA oligonucleotide splint molecule comprising a nucleotide sequence complementary to the second right primer readout portion (rs3) nucleotide sequence.

Clause 85. The composition of any one of clauses 80-84, further comprising an intermediate padlock bottom-binding oligonucleotide molecule comprising (i) a first intermediate primer readout portion (rs6); (ii) a first intermediate primer readout portion (rs5); and (iii) an amplicon padlock bottom barcode portion (aH2) comprising a nucleotide sequence complementary to the second targeted nucleotide sequence (H2′).

Clause 86. A kit comprising the composition of any one of clauses 80-85, optionally further comprising a DNA ligase and/or a DNA polymerase.

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

### General

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1—Detecting Nucleotide Sequences in Cells

In the broadest sense, an approach to detect the presence of a particular nucleotide sequence is described herein. In particular, the approach is amenable for the detection of nucleotide sequences that make up biomolecules such as DNA and RNA transcripts in biological samples such as cells.

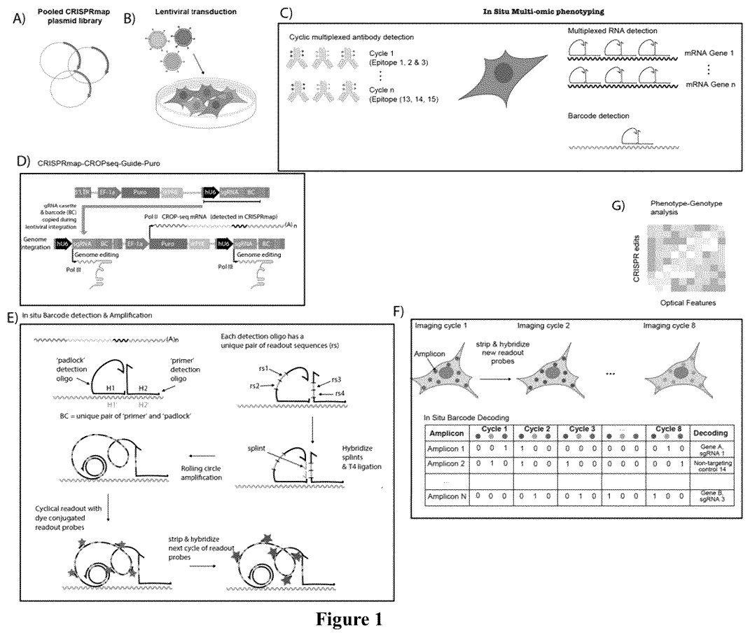

The nucleotide sequence of interest is detected by a pair of single-strand DNA oligos (here called a padlock oligo and a primer oligo, FIG. 1E). The padlock and primer oligo encode a sequence (H1 and H2 respectively) that is complementary to the target nucleotide sequence (H1′ and H2′ respectively, where H1′ is the reverse complement of H1), and correct detection occurs if both oligos hybridize to their respective, adjacent, complementary sequences (FIG. 1E).

Unlike the approach in Wang et al. “Three-dimensional intact-tissue sequencing of single-cell transcriptional states” Science (2018) Jul. 27; 361 (PMID: 29930089), we ensure that information carried by the primer oligo gets incorporated into the circularized padlock probe by hybridization of complementary oligos, which we call splints (See e.g., FIG. 1E). This enables verification at the readout level (discussed later) that the signal was generated by the desired pair of primer padlock probes.

In an approach presented herein, the primer and padlock detection oligos and the splint oligos have different functions and are all required to yield amplified signal. They thus collectively function as an AND function, in the sense that padlock, primer and splint oligos need to hybridize to their respective target sequences, in order for the primer to serve as a template for the circularization of the padlock probe. Once the padlock and split oligos have been ligated, the primer oligo can initiate rolling circle amplification (RCA). This AND functionality safeguards the specificity of the detection, as otherwise any non-specifically bound padlock oligo, or a padlock probe primed by an incorrect primer oligo, or any other nucleotide resident in the cellular environment, could serve as ligation template and/or RCA primer to yield false positive signal.

A second difference with Wang et al. and equivalent approaches such as Feldman et al. “Optical Pooled Screens in Human Cells” Cell (2019) Oct. 17; 179(3):787-799 (PMID: 31626775) is that we do not use sequencing for readout, which can suffer from low transcript detection efficiency (see e.g., Ke et al. “In situ sequencing for RNA analysis in preserved tissue and cells. Nat Methods (2013) September; 10(9):857-60, PMID: 23852452; and Qian et al. “Probabilistic cell typing enables fine mapping of closely related cell types in situ. Nat Methods (2020) January; 17(1):101-106, PMID: 31740815) due to several factors, including enzymatic steps and the need to perform in situ cDNA synthesis in some cases. In addition, often a universal sequencing primer is used which means all amplicons are sequenced simultaneously. This can limit the number of amplicons that can be identified in a given cell or tissue volume as optical crowding can become problematic. Optical crowding occurs when two amplicons are too close together to be optically resolved due to resolution limits of the assay. This can become problematic when developing an assay that aims to read out transcripts of hundreds to thousands of genes for a given cell or tissue section, severely limiting the number of RNA molecules that can be correctly detected and decoded, and thus the accuracy of the cellular state that is obtained.

Moreover, in-situ sequencing by ligation reactions (see Wang et al., PMID: 29930089) requires multiple cycles of staining with high levels of fluorophore coupled ligation oligos (uM range), which are expensive, and the high concentration contributes to non-specific background signal. Furthermore, the ligation process is mediated by enzymes, which are costly, and require long incubation times to ensure satisfactory levels of ligation, and thus signal generation for readout. In addition to the added cost for the multiple cycles, the long incubation limits the throughput of the assay, limiting the number of samples one can profile.

Our readout approach instead utilizes a combinatorial hybridization of four (4) readout sequences per primer/padlock pair over the course of multiple imaging cycles (FIG. 1E, bottom, FIG. 1F). Multiplexed FISH approaches (see Chen et al. “Spatially resolved, highly multiplexed RNA profiling in single cells” Science. 2015 Apr. 24; 348, PMID: 25858977; and Shah et al. “Dynamics and Spatial Genomics of the Nascent Transcriptome by Intron seqFISH” (Cell) 2018 Jul. 12; 174(2):363-376, PMID: 29887381) have never been applied in the context of RCA. The signal gain associated with RCA enables for higher throughput imaging, thus enabling higher throughput screening, or higher throughput profiling of tissue sections.

A different approach called HybRISS (Lee et al. “Direct RNA targeted in situ sequencing for transcriptomic profiling in tissue” Sci Rep. (2022) May 13; 12(1):7976, PMID: 35562352) performs RCA and read out by hybridization of a 20mer that is conjugated to a dye. In contrast, however, HybRISS does not use an AND logic dual detection oligo approach, and it has a unique readout probe per target gene, thus not making use of combinatorial readout schemes to minimize the number of readout cycles for large target panels envisioned in screening efforts or highly multiplexed transcriptomics.

Currently, we have applied and validated our approach to two distinct use cases: detection of cellular barcodes, and multiplexed detection of RNA transcripts.

### Readout of Cellular Barcodes

We leverage the ability to detect specific adjacent sequences on RNA transcripts to barcode cells. Specifically, our barcode consists of a unique pair of adjacent sequences, which are part of an mRNA sequence expressed in the barcoded cells. The sequences that make up the barcode are designed to have 1) minimal similarity to the mouse and human genome and transcriptome, 2) minimal secondary structure, 3) similar melting temperatures among all primer and padlock hybridization sequences used in the assay.

In particular, pooled CRISPR perturbation approaches have utilized pooled lentiviral libraries to infect cells. If the multiplicity of infection is kept low (MOI −0.1), most cells that receive a perturbation are only perturbed for a single gene. Which gene of the pool any given cell is perturbed for is identified by readout of the cellular barcode. Pooled oligos are synthesized, where each oligo in the pool contains a CRISPR guide and a corresponding barcode that identifies the guide encoded by that oligo. Pools can be synthesized for hundreds to thousands of different oligos (and thus thousands of different CRISPR guides), enabling large scale CRISPR perturbation studies.

We PCR amplified the synthesized pool of oligos, and performed NEBHIFI cloning into a modified cropseq vector (see Datlinger et al. “Pooled CRISPR screening with single-cell transcriptome readout” Nat Methods (2017) March; 14(3):297-301, PMID: 28099430, which describes that “the gRNA becomes part of the puromycin-resistance mRNA transcribed by RNA polymerase H, and functional gRNAs continue to be expressed from the hU6 promoter. In addition, the entire hU6-gRNA cassette is copied to the 5′LTR during reverse transcription and integration of the virus. This results in a second copy upstream of the possibly interfering EF-la promoter.”)

We modified the CROPSEQ v2 plasmid (addgene plasmid 127458, Feldman et al., PMID: 31626775), to remove the guide scaffold from the vector and make it part of the oligo pool construct to enable a separation of the guide expression (terminated by a POLIII terminator sequence) and the barcode to ensure the gRNA is unaltered enabling optimal CRISPR editing. The barcode is expressed as part of the puromycin-resistance mRNA transcript expressed from an EF-la promoter. (FIG. 1D).

Next, we transformed the pool of plasmids in MegaX DH10B T1R Electrocomp Cells, and grow them up at a scale such that each guide is represented by, on average, by at least 300 colonies. Subsequent lentiviral production generated supernatant with lentiviral particles, which were used to infect cells at an MOI of −0.1.

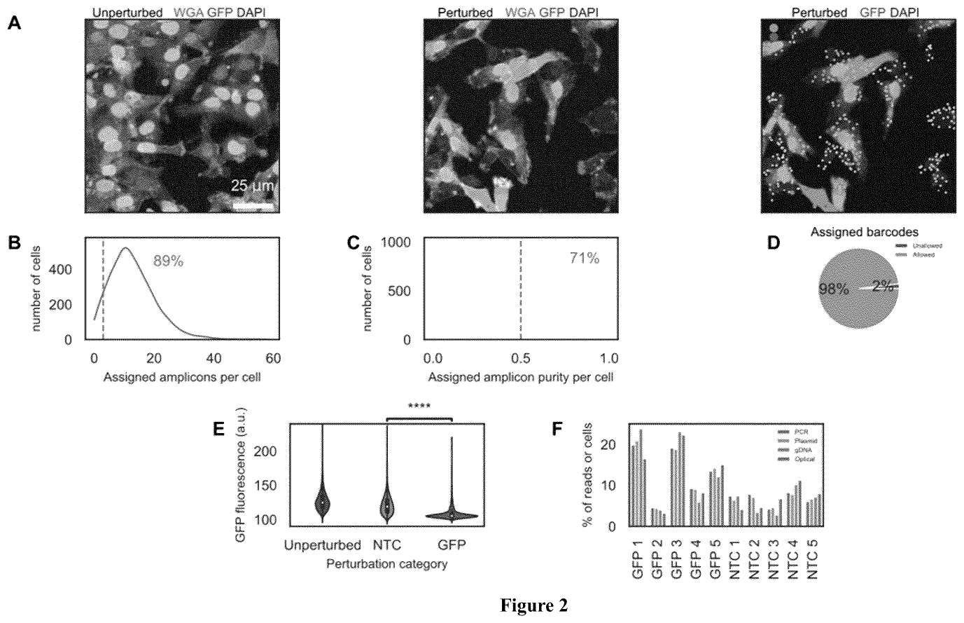

We further transduced a small pilot lentiviral library containing five GFP targeting CRISPR guides, and five non-targeting CRISPR guides in a HT1080-Cas9 cell line expressing copGFP. We performed lentiviral library preparation and infected cells at an MOI of 0.1 to ensure most infected cells will be edited by a single guide, and expressed a single barcode after puromycin selection. A first imaging round visualized DAPI, GFP and WGA (FIG. 2A). The WGA and DAPI signal are used for cell and nuclear segmentation respectively. To couple our optical phenotype (GFP expression) to the CRISPR edit, we performed our assay, which we call CRISPRmap.

For cellular barcoding our primer oligo encodes for (from 5 prime to 3 prime): a sequence to hybridize to the barcode carrying mRNA, a universal sequence for the 5′ of the padlock probe to hybridize to, followed by two 20mer sequences to hybridize splints to, followed by a universal sequence for the 3′ end of the padlock probe to hybridize to. In this scenario, each primer oligo is uniquely defined by its hybridization sequence to the barcode target on the mRNA, and by a unique pair of splints. Although it should be noted that any given splint hybridization sequence can be used for multiple primer oligos (i.e. only the combination of splint hybridization sequences is unique to any given primer).

Similarly, a padlock probe encodes for (from 5 prime to 3 prime): a universal sequence that hybridizes to the primer oligo, a sequence to hybridize to the barcode carrying mRNA, followed by a pair of 20mer readout sequences, followed by a universal sequence to hybridize to the 3′ end of the primer oligo. In this scenario, each padlock oligo is uniquely defined by its hybridization sequence to the barcode target on the mRNA, and by a unique pair of readout sequences. Although it should be noted that any given readout sequence can be used for multiple padlock oligos (i.e. only the combination of readout sequences is unique to any given padlock oligo).

After splint hybridization to the primer oligo, we perform a DNA ligase reaction that circularizes the padlock oligo to incorporate the two splint oligos, we will call this circularized oligo the amplicon template oligo. The amplicon template oligo carries 4 readout sequences that are unique to the primer-padlock oligo pair and thus identify the barcode sequences the primer and padlock hybridized to.

The amplicon template oligo is copied through rolling circle amplification, primed by the primer oligo. The product of this RCA reaction we call amplicons.

The four (4) readout sequences on any given amplicon are identified optically by cyclic hybridization rounds whereby complementary 20mer readout probe oligos that are conjugated to dye molecules hybridize to their target readout sequence.

An example library of 54 primer and 54 padlock oligos can encode for up to 2916 barcodes, which can be imaged in 3 channels over 8 imaging cycles. We can have each readout set (combination of 4 readout sequences) consist of readout sequences that are read out in different channels, which further minimizes the number of imaging cycles needed for a given amount of barcodes, thus contributing to the throughput of the assay. This is enabled by the strength of RCA signal, enabling computational alignment of readout cycles across images taken in different channels.

Moreover, we have designed our approach such that cells (or tissue sections) are plated on glass bottom well plate wells. This enables easy handling of samples (either by hand or automated liquid handlers), minimizes staining and enzymatic volumes needed for reactions (no dead volume in microfluidic lines), and enables samples to be removed from the microscope during cyclic staining steps, which minimizes microscope downtime, and thus contributes to assay throughput (i.e. one can work with multiple glass bottom plates and alternate plates such that the microscope is always occupied).

Between imaging rounds we observe small global translational shifts (i.e. misaligned glass bottom well plate placement) as well as local translational shifts (i.e. cells slightly shifting between imaging rounds). To align the images across all imaging rounds we calculated the transformation matrices for each round using the TV-L1 implementation of optical flow on binary nuclei masks derived from DAPI stains.

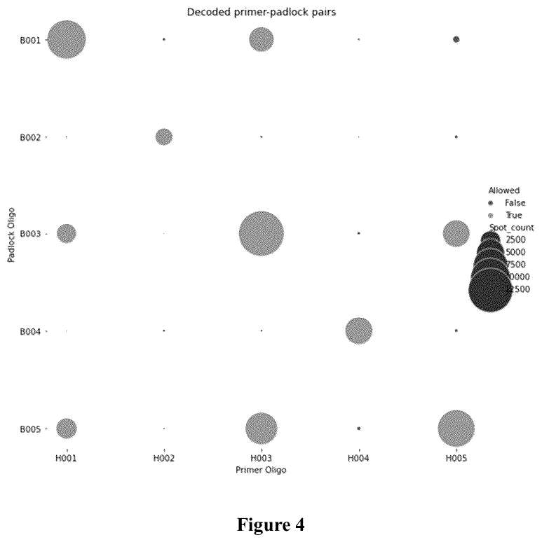

Amplicon locations were identified as local maxima of a summed image across all barcode readout rounds and channels. For each amplicon location, the pixel intensities of the raw image for all barcode readout rounds and channels were compared to a user defined threshold and the amplicon was marked positive (1) if the pixel intensity exceeded the threshold and as 0 otherwise. As such, each amplicon generates a 24 bit code (FIG. 1F). This code is mapped against the codebook used when designing the library to assign an amplicon to their corresponding guide ID. In our GFP targeting dataset, we found that of the amplicons that were positive for 4 readout probes, 98% coded for a 24 bit code that was part of the library design, whereas 2% of amplicons reported an unallowed readout set (FIG. 2D). To visualize that the vast majority of amplicons detected were initiated by an allowed primer and padlock combination, we plotted a primer-padlock grid where the spot at each node of the grid is sized according to the frequency of observation and colored orange if it is an allowed primer-padlock combination and blue if it is an unallowed combination (FIG. 4). From a per-cell analysis, we found that when imaging with a 20× objective, the median number of guide assigned amplicons per cell was 12 (FIG. 2B), and the median guide purity per cell was 0.8 (FIG. 2C). We restricted further analysis to cells with 4 or more amplicons and a guide purity above 0.67.

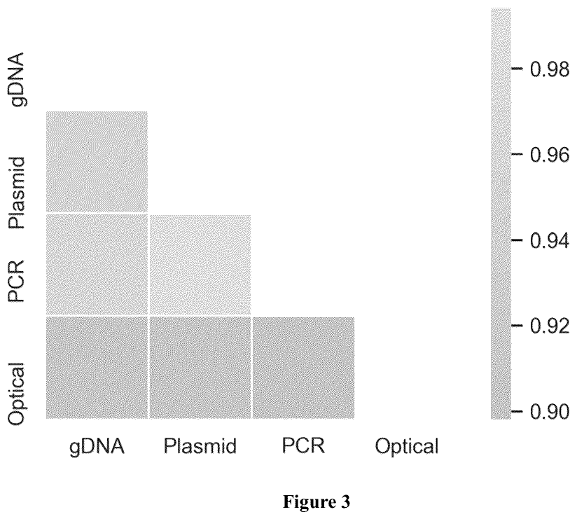

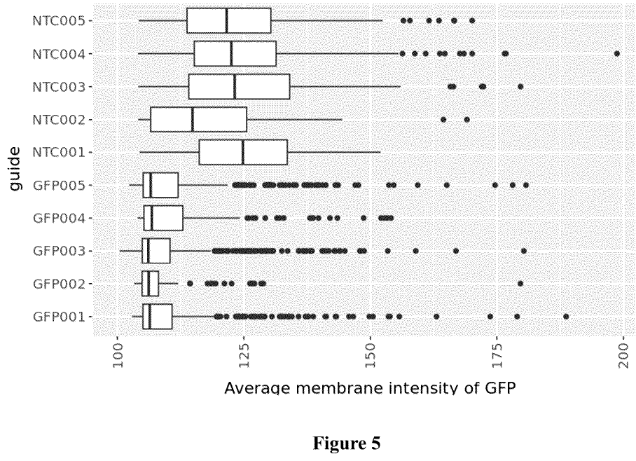

To assess guide representation throughout library preparation, infection and optical readout of the barcodes, we performed NGS sequencing on PCR product of the amplified opool, the CROPseq plasmid pool and the genomic DNA from the cells. We observed highly correlated guide frequencies between all of these stages (FIG. 2F, & FIG. 3). Finally, we evaluated if we observe the expected optical phenotype for each of the guides in our pilot library (GFP knockout for GFP targeting guides, and GFP fluorescence for NTC guides), and found indeed that GFP targeting guides have significantly lower GFP fluorescence levels than NTC guides (FIG. 2E), which in turn have similar GFP fluorescence levels as control WT cells. Each of the five GFP targeting guides showed significantly lower GFP levels than any of the NTC control guides (FIG. 5).

### Expanding to Genome-Wide Screens

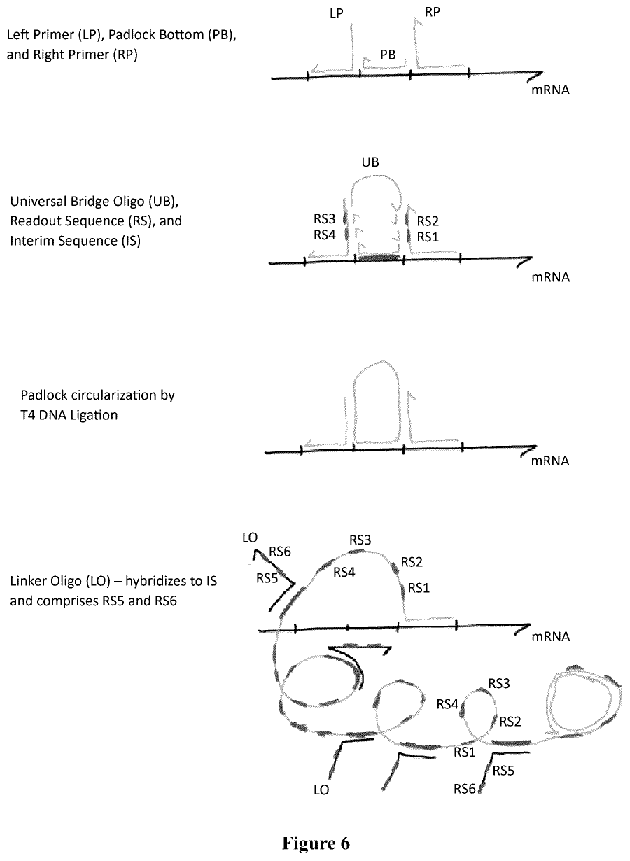

For larger scale screens (tens to hundreds of thousands of barcodes), we have adapted our assay to guard for high throughput. In particular we have increased the number of adjacent hybridization sequences that make up the barcode from 2 to 3. In accordance, we have three (3) detection oligos, which we term left primer, padlock bottom, and right primer (FIG. 6). Each of the primers encodes for hybridization sequences for two (2) splints, similar as described before. The padlock bottom, splints, and a universal ‘bridge’ oligo enable circularization by DNA ligation to form an amplicon template oligo. The readout sequence set for a given barcode consists of a unique combination of six (6) readout sequences, four (4) of which are encoded by the splints, and the remaining two (2) are provided by an intermediate oligo that hybridizes to the padlock bottom sequence and carries a pair of readout sequences to be detected by the remaining two (2) readout sequences.

For instance, for a human genome wide screen, targeting −25 k genes with three (3) guides each would require >75 k barcodes. Our assay utilizing 45 detection oligos of each of the primers and padlock bottom would encode for 45³, or 91,125 barcodes, which can be read out by 30 readout probes in 10 imaging cycles profiling 3 channels in each cycle. (Each of the primer/padlock bottom oligos is read out by two (2) readout probes. Since 10 choose 2 yields 45, we need 10 readout probes per detection oligo class (i.e. left primer, padlock bottom, and right primer).

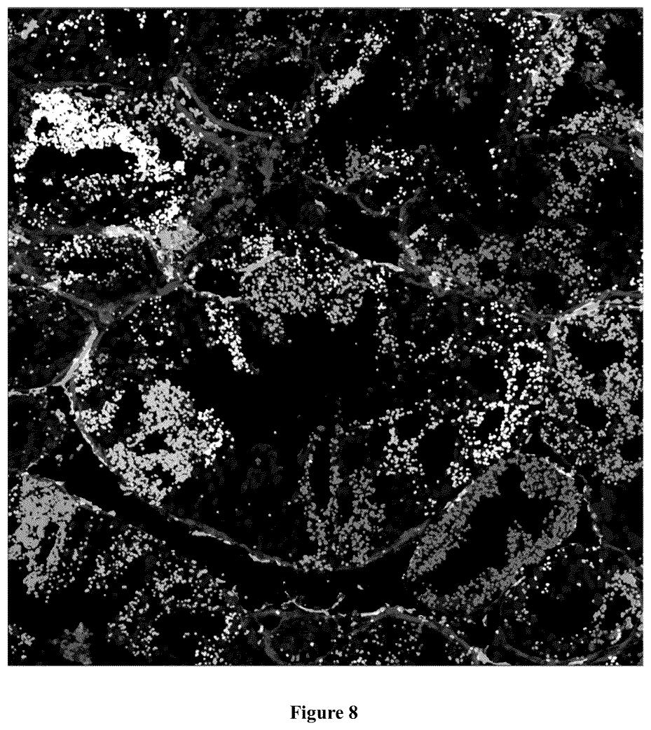

We have validated that this approach generates similar amplicons as our initially developed assay, with uniform amplicons being reported per cell. (FIG. 8).

Our approach is widely applicable as demonstrated by the readout of barcodes in the following cell lines: HT1080 fibrosarcoma cell line), MCF7 (human breast cancer cell line), U-2 OS (human sarcoma of the tibia, epithelial), 0E19 (human adenocarcinoma of gastric cardia/oesophageal gastric junction), SW620 (human colorectal cancer cell line), and A375 (human malignant melanoma), to name a few of the cell lines tested.

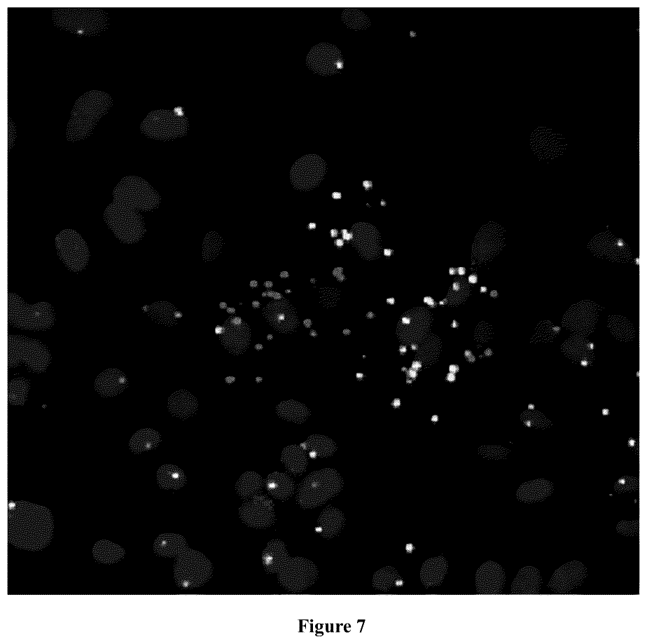

Crucially, we have demonstrated our approach to be applicable in a tissue context as well, which to date has not been reported. Evaluating cellular behavior in a native tissue context is crucial as it allows us to take into account crucial components to cellular function such as interactions with extracellular matrix, interaction with the immune system of the organism, and intercellular communication patterns that cannot be recapitulated in invitro or organoid settings. Specifically, we performed a subcutaneous injection of edited 0E19 cells in nude mice, allowed for 14 days of tumor growth, and applied our assay on sections of the isolated tumor tissue (FIGS. 7 & 8). Importantly, we observe growth of epithelial ring structures as expected, and moreover, these ring structures are often made up of cells with the same barcode, indicating that a single cell is likely to give rise to this biologically relevant structure. It also validates the specificity of our barcode detection, as random recapitulation of such a structure is highly unlikely. Crucially, our assay provides resolutions associated with microscopy, and thus enables down to 200 nm resolution if needed, enabling subcellular readout of abundance and localization patterns of proteins. This resolution is crucial in evaluating cell-to-cell interaction mechanisms, and cellular and tissue organization patterns in health and disease, and how perturbations affect them. Typically, current approaches that profile transcriptomic cell states and their perturbations lack single-cell resolution obfuscating the analysis of tissue organization and intercellular communication patterns.

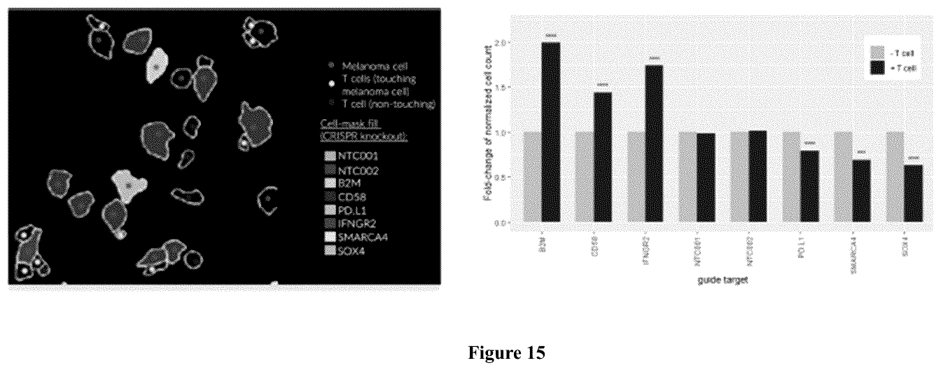

In addition, we have demonstrated our approach can be used in patient-derived melanoma cells and autologous tumor infiltrating lymphocyte (TIL) co-cultures, to evaluate Tcell mediated killing as a function of genes perturbed in the melanoma cells. (FIG. 15) As expected, when evaluating the susceptibility of a melanoma cell to be lysed by T-cells, we observed no differences for non-targeting controls when comparting cell frequencies between Tcell positive and negative co-cultures. In contrast, when knocking out B2M, CD58 and IFNGR2, we observe that these melanoma cells are relatively speaking less likely to be killed by T-cells as these genes are needed for optimal T-cell mediated lysis. If, on the other hand, we knock out known therapeutic targets such as PD-L1, SMARCA4 or SOX4, cells become more susceptible to T-cell mediated killing.

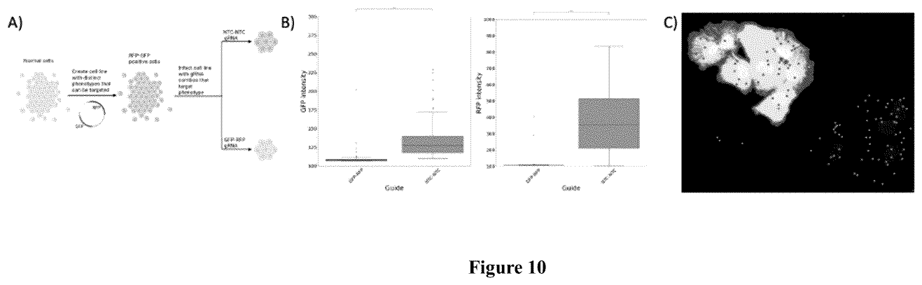

Moreover, we have leveraged our barcode readout approach to study genetic interactions by combinatorial CRISPR perturbation. To this end, we sought to validate efficacy of EnAsCas12a mediated double knockout. To expedite assay development turn-around time, we developed this in a melanoma cell line (A375), and generated a cell line with an immediately recognizable optical phenotype (GFP and RFP expression, FIG. 10A). Next, we generated a lentiviral library that contained guide arrays consisting either of 2 pairs of non-targeting CRISPR guides (NTC-NTC), or a pair of GFP targeting and a pair of RFP targeting guides (GFP-RFP). As expected, cells identified to express the GFP-RFP guides had significantly lower values of GFP and RFP when compared to NTC-NTC cells (FIGS. 10B & 10C).

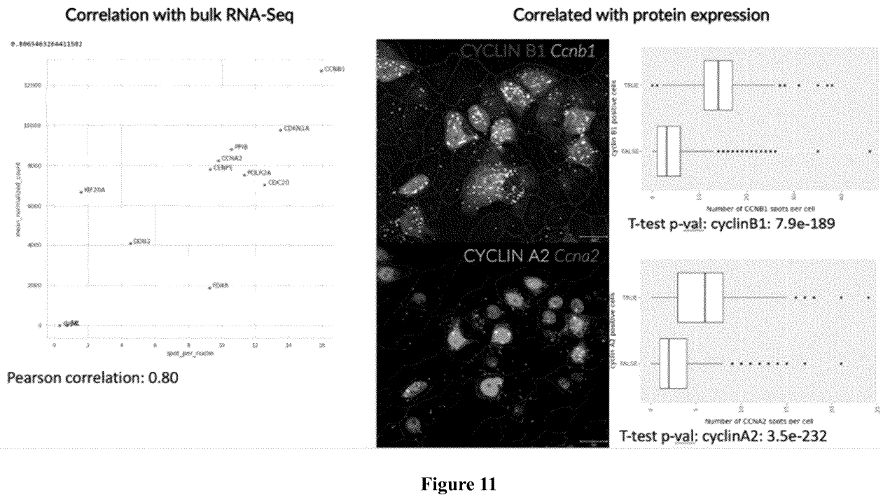

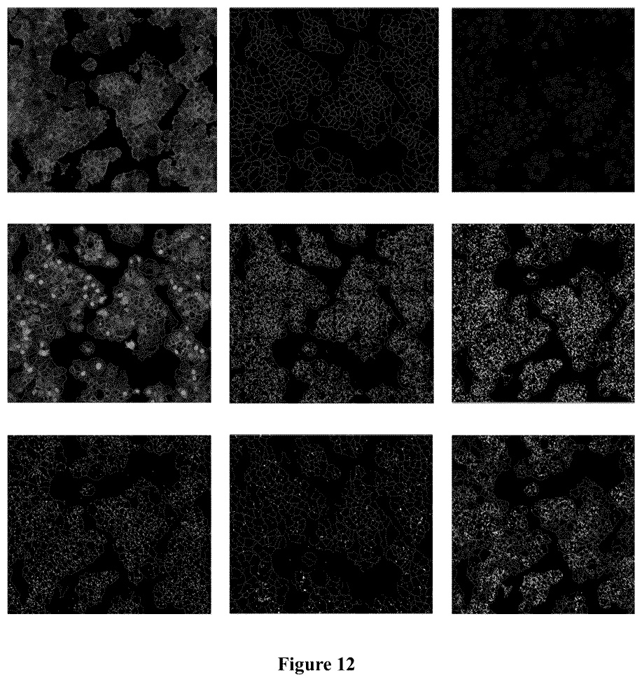

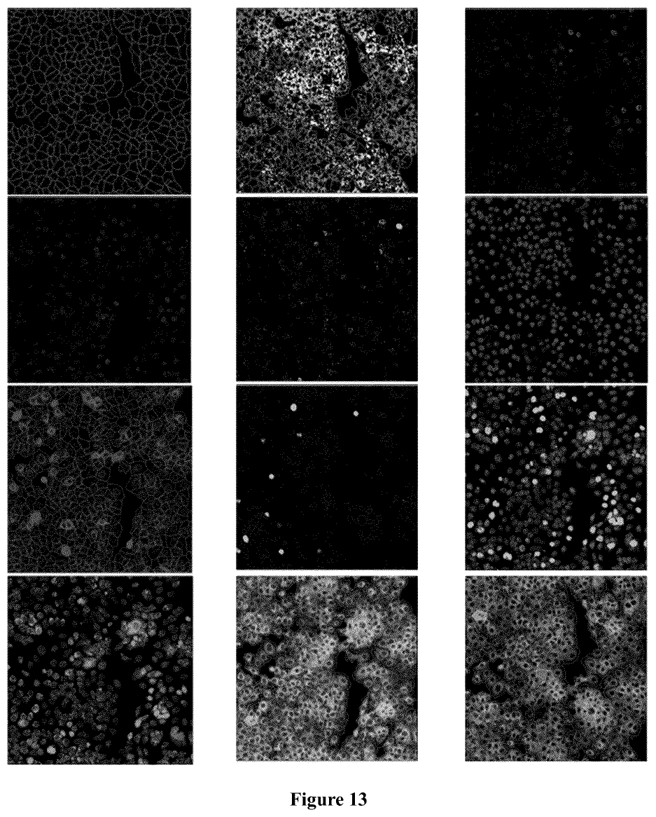

In addition, our assay is compatible with multi-omic readout. For instance, we have coupled barcode readout with multiplexed transcriptomic readout (FIG. 11 & FIG. 12), and with cyclic IF readout (FIG. 11 & FIG. 13). It has become clear that evaluating multi-omic cell states is crucial for evaluation cellular behavior, and the ability to evaluate them as a function of CRISPR perturbations will enable us to further dissect cellular function in physiological cellular function and disease.

Moreover, we have validated correlation of mRNA expression with bulk RNA-sequencing by profiling 12 genes in situ (FIG. 11), and by evaluating the correlation between protein and mRNA levels measured at the single-cell level optically (FIG. 11), and we indeed see statistically significantly differences in mRNA detection for cells that stained positive for antibodies against Cyclin A2 and Cyclin B1 (FIG. 11).

### Transcriptome Profiling

In recent years, efforts are underway to profile the transcriptomic states of cells in the native tissue environment. To profile cellular states, high-multiplex approaches have been developed recently to measure multiple genes simultaneously (Chen et al., PMID: 25858977; Shah et al., Ser. No. 29/887,381; Lee et al. 35562352).

Our approach was developed to detect RNA transcripts in a similar way as the above barcodes. Instead of pre-designed hybridization sequences, our primer and padlock detection oligos now target adjacent sequences on the target RNA molecule. To ensure specificity, we select hybridization sites that are target specific by minimizing off-target alignment during a BLAST search in the reference genome and transcriptome, we minimize secondary structure of the target and detection probes, and we optimize the melting temperature of the hybridization sequence to fall within a desirable range for uniform detection of all transcripts in the target pool. Any given RNA target is typically detected by 4 to 8 pairs of primer/padlock oligos, where the target hybridization sequences are unique for each pair, but the readout sequences are shared among the target specific oligo pairs. For RNA detection we have slightly changed the padlock oligo to contain 3 readout sequences, and the primer oligo to provide a single hybridization to a splint oligo (instead of 2 splints in the barcoding scheme). This change was implemented to enable readout chosen to be part of the panel to be hamming distance 4 apart from each other to enable error correction (discussed below).

Specifically, our identifying readout sequence consists of 4 20 bp hybridization sites (discussed below). One of these hybridizations sequences is not contained in the padlock probe, but is part of the primer encoding probe instead. Upon adjacent binding of padlock and primer probe, the 5′ and 3′ end of the padlock probe are designed such that they hybridize to the primer probe, but the 5′ and 3′ ends are separated by a 24 bp gap. On the primer oligo, this stretch of 24 bases encodes a central 20 bp hybridization sequence, flanked by 2 universal bases on either end to have uniform and optimal ligation efficiency for T4 DNA ligation. After primer and padlock hybridization, a second hybridization step is performed with a pool of splint oligos. Thanks to the high sequence specificity of the hybridization sequence this staining is done at nanomolar concentration levels (−5 nM for 30 minutes), yielding high specificity. Padlock probes that were ligated non-specifically, or by a non-specific primer probe will either lack the 4^{th} readout bit, which allows us to identify it as non-specific signal and reject it for subsequent analysis. Padlock probes that might have been primed by a non-specific primer oligo will have a 4^{th} readout bit, but it is most likely to yield an invalid codeword, and can thus be identified as non-specific signal and rejected for subsequent analysis. For instance, if we utilize a 36 bit code, genes will carry one of 9 (36/4) 4^{th} readout bits, and non-specific primers will carry the wrong 4^{th} bit—89% (8/9) of the time. Expanding our assay to a 80 bit code (as done for SeqFISH) could increase this identification rate to 95% (19/20).

Our approach pioneers the use of combinatorial FISH readout of RCA amplicons. Fluorescent in situ hybridization readout has the benefit of being able to stain at nanomolar ranges of fluorescent detection oligos (i.e. 5 nM), which is 3 orders of magnitude lower than the uM staining of SBL methods, and thus significantly decreases non-specific background signal, and utilizing far less costly fluorescently conjugated oligos. Moreover, the hybridization kinetics of a 20 bp readout probe are faster, no enzymatic steps are needed, increasing throughput and minimizing cost.

The combinatorial set of hybridization sequences that identifies a particular RNA target is similar to the MERFish approach (Chen et al., PMID: 25858977), bestowing our assay with error correction capability (as all code words are at least a Hamming distance of four (4) apart), and the crucial ability dilute the number of RNA species to be detected during any given hybridization cycle by expanding the number of readout cycles, thus providing an experimental handle to minimize optical crowding.

We have opted to use rolling circle amplification, which has shown to yield high levels of amplification, to the extent that it enables us to tweak the level of amplification to optimize it to the desired level of amplification/readout. For instance, if a study aims high throughput scanning of large areas of tissue for many tissues, we can increase the amplification time to generate longer amplicons, thus requiring lower magnification objectives to obtain a given S/N and improving the area of tissue one can image per time unit.

We have validated our approach in mouse liver tissue by profiling three (3) genes with distinct expected spatial patterns known from literature, namely Albumin and Pckl, which are expressed by hepatocytes and are thus expected to expression in the same cell. And Glul, which has been found expressed in endothelial cells, particularly those lining central veins.

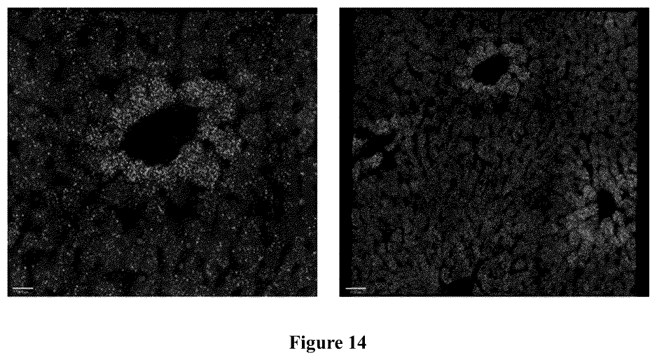

Our data does indeed recover these expected spatial patterns with excellent signal-to-noise levels (FIG. 14).

### Example Protocol

Probe Hybridization on MCF7-BE3 Cells with Ionizing Radiation

MCF7-BE3 cells were seeded onto 6-well glass bottom plates (Cellvis P06-1.5H-N) at a density of 50,000 cells/cm². After 48 hours, cells were exposed to 10Gy ionizing radiation. Six (6) hours after radiation, cells were fixed with 4% PFA (Electron Microscopy Sciences 15710) in PBS for 10 minutes at room temperature and rinsed twice with PBS. Cells were then permeabilized with 0.1% Triton-X100 (Sigma-Aldrich T8787) in PBS for 10 minutes on ice, and rinsed twice with PBS. After permeabilization, the cells in each well were incubated in 1 mL of the first-round antibody mix (2 ug/ml anti-CD326 (BioLegend 312502), 1 ug/ml anti-RAD51-AF647, 1 ug/ml anti-BRCA1-AF555, 1 ug/ml anti-RPA2-AF488 in PBS) for 1 hour at room temperature, rinsed three times with PBS, then incubated in 10 ug/ml Goat anti-Rat-IgG secondary antibody (Thermo SA5-10023) for 30 minutes at room temperature, and rinsed three times with PBS. 4% PFA in PBS was then added for 10 minutes at room temperature to cross-link the antibodies to the cells, followed by three rinses in PBS.

The cells in each well were incubated in 1 mL of the Hybridization mix (3 nM CRISPRmap Head mix, 3 nM CRISPRmap Body mix, 3 nM RNAMap Head mix, 3 nM RNAMap Body mix, 0.1% yeast tRNA (Invitrogen 15401011), 2×SSC, 20% formamide in ultrapure water) for 16 hours at 40° C. After hybridization, the cells were first rinsed three times with the formamide wash buffer (2×SSC, 15% formamide in ultrapure water) and then incubated in the formamide wash buffer for 5 minutes at 40 C for three times. The cells were then incubated in 1 mL of the splint mix (10 nM CRISPRmap splint mix, 0.1% yeast tRNA in 2×SSC, 15% formamide) for 30 minutes at 37 C and rinsed twice with the formamide wash buffer.

### T4 DNA Ligation

After probe hybridization, the cells were first incubated in 2×SSC for 15 minutes at room temperature. Then, cells in each well were incubated in 1 mL of the ligation mix (lx T4 ligase buffer, 1% T4 DNA ligase (Enzymatics L6030-HC-L) in ultrapure water) for 2 hours at 16 C and 1 hour at 25 C. Cells were rinsed twice in PBS.

### Rolling Circle Amplification

After DNA ligation, the cells in each well were incubated in 1 mL of the rolling circle amplification mix (1× Qualiphi buffer, 2% v/v QualiPhi DNA Polymerase (4basebio, 510100), dNTP mix 0.25 mM each (Thermo R1122), 0.02 mM 5-(3-Aminoallyl)-dUTP (Thermo AM8439) in ultrapure water) for 8 hours at 30 C, then immediately fixed with 4% PFA in PBS. Cells were rinsed three times in PBS.

### Readout Probe Hybridization & Stripping

The cells in each well were incubated in 1 mL of the readout probe mix (10 nM if each readout probe, 2×SSC, 15% formamide in ultrapure water) for 30 minutes at 37 C. Cells were then incubated in the imaging buffer (0.5 ug/ml DAPI (abcam ab285390), 10 ug/ml Fungin (InvivoGen ant-fn-1) in PBS). After imaging, the cells were incubated in the stripping buffer (2×SSC, 50% formamide in ultrapure water) for 30 minutes at 40 C then rinsed once with the formamide wash buffer.

### Example 2—Mapping Multi-Modal Phenotypes to Perturbations in Cells and Tissue with CRISPRmap

### Summary

Unlike sequencing-based methods, which require cell lysis, optical pooled genetic screens enable investigation of spatial phenotypes, including cell morphology, protein subcellular localization, cell-cell interactions and tissue organization, in response to targeted CRISPR perturbations. Here we report a multi-modal optical pooled CRISPR screening method. CRISPRmap combines in situ CRISPR guide-identifying barcode readout with multiplexed immunofluorescence and RNA detection. Barcodes are detected and read out through combinatorial hybridization of DNA oligos, enhancing barcode detection efficiency. CRISPRmap enables in situ barcode readout in cell types and contexts that were elusive to conventional optical pooled screening, including cultured primary cells, embryonic stem cells, induced pluripotent stem cells, derived neurons, and in-vivo cells in a tissue context. We conducted a screen in a breast cancer cell line of the effects of DNA damage repair gene variants on cellular responses to commonly used cancer therapies and show that optical phenotyping pinpoints likely pathogenic patient-derived mutations that were previously classified as variants of unknown clinical significance.

### Introduction

Pooled CRISPR screens, where the responses of many individual cells to different genetic perturbations can be measured in parallel, are enabling an increasing variety of high-throughput genetic analyses. All such studies must correlate phenotype with the specific genetic modification, which is generally identified by the readout of a DNA encoded barcode. This was originally achieved for assays of cell viability or expression of a marker used for cell sorting by measuring enrichment or depletion of specific barcodes from the bulk or sorted cell population. To allow screening for molecular phenotypes it is necessary to measure these parameters and associated barcodes in single cells. Coupling single-cell RNA-sequencing (scRNA-seq) to pooled CRISPR screens has vastly expanded our ability to study the transcriptomic response to perturbations^{1,2}, but suffers from the necessity to isolate and lyse cells. As such, scRNA-seq approaches are agnostic to spatial organization of inter- and intracellular phenotypes. Imaging techniques have emerged to enhance these screens³⁻⁶, capturing complex cellular behaviors and dynamic phenotypic changes, including intricate cellular morphology and molecular distribution, without destroying the cells. These advancements make it possible to observe a wide array of spatially resolved cellular phenotypes in genetic screens.

The integration of single-cell multi-modal profiling, which concurrently analyzes proteins and RNA, is crucial for a nuanced understanding of cellular function. While RNA profiling provides data on gene expression, sole reliance upon RNA profiling can be fraught with incomplete or erroneous conclusions due to post-transcriptional and post-translational processes that RNA sequencing is blind to. Combining multimodal profiling with the ability of optical pooled CRISPR screens to perturb at a pathway- or even genome-wide scale, has not yet been widely adopted, despite its potential to expand our understanding of how cellular pathways are regulated in health and disease states like cancer.

Pooled base editor screens have recently emerged as a powerful method for mutational scanning, enabling researchers to directly alter endogenous proteins within live cells and thereby revolutionize the study of proteins in their natural environments^{7,8}. These screens can use nuclease hybrids of deficient Cas9 with APOBEC1 (BE3) guided by single guide RNAs (sgRNAs) to induce specific point mutations through direct chemical modification, offering a precise means of editing⁹. These precise base changes largely occur within a defined genomic nucleotide window¹⁰. The precision of editing is advantageous for high-resolution analysis of protein function, and the mapping of sequence-activity relationships.

Leveraging multi-modal optical pooled screens to interrogate key cellular and clinically relevant pathways holds immense potential. Indeed, as next-generation sequencing has become more common in clinical oncology, there has been an increasing number of variants of unknown significance (VUS) in genes linked to cancer predisposition and aggressiveness¹¹. In particular, VUS are commonly identified in DNA damage response (DDR) genes, which are critical for genomic stability, DNA damage signaling, DNA-damage related checkpoints, and DNA repair genes¹². The importance of understanding whether a VUS is functional or a passenger event is underscored by real-world clinical consequences. For example, patients with particular VUS may be a candidate for therapy leveraging impairment of a DDR pathway or whether relatives may be at elevated cancer risk if such a mutation arises in the germline. As such, understanding the normal function of each gene and how key mutations alter homeostasis is critical. To disentangle the phenotypic difference among these variants during DNA damage repair, we applied five different treatments to MCF7 breast cancer cells to introduce DNA damages through different mechanisms of action. Ionizing irradiation directly introduces DNA double-strand breaks to the target cells. Camptothecin specifically inhibits the DNA topoisomerase I, causing replication fork collisions¹³ Olaparib targets the poly(ADP-ribose) polymerase to blockade the repair of single-strand DNA breaks which results in DNA double-strand breaks during replication¹⁴. Cisplatin causes interstrand crosslinks by crosslinking the purine bases on the DNA¹⁵. Etoposide introduces DNA double strand breaks by targeting the Topoisomerase II¹⁶. These DNA damaging agents are used clinically, and the variant specific responses to drugs thus holds potential to help prioritize therapeutic strategies. Furthermore, due to the essential nature of many DDR genes for cell viability, efforts to genetically deplete DDR genes may not recapitulate clinically-observed variants and function. Hence, efforts to interrogate the function of these proteins with point mutations is necessary to understand their function.

Ideally, the effects of variants, or any genomic perturbation, would be studied in the native context the cell encounters in vivo. Recent technological advancements have allowed for protein epitope-based identification of CRISPR guide expressions within tumor tissues at a single-cell resolution¹⁷. RNA-based barcoding holds the promise to increase the complexity of these libraries, but its application in tissue contexts has not yet been reported.

Building on these innovations, we have developed a sequencing-free barcode readout approach for optical pooled CRISPR screens that is compatible with highly multiplexed antibody and RNA transcript profiling. We have applied this method to a breast cancer cell line to evaluate how 292 nucleotide variants across 27 key DDR genes affect the DNA damage response by visualizing the recruitment of DDR proteins to sites of DNA damage during different cell cycle phases after ionizing radiation exposure. Our work also demonstrates the capability to optically read RNA-encoded barcodes in tissue sections, linked with multiplexed antibody detection. This serves as a stepping stone toward in vivo CRISPR screens that can map the cellular landscape and pathway behaviors at a subcellular level.

### Results

### CRISPRmap Enables Optical Readout of Cellular Barcodes

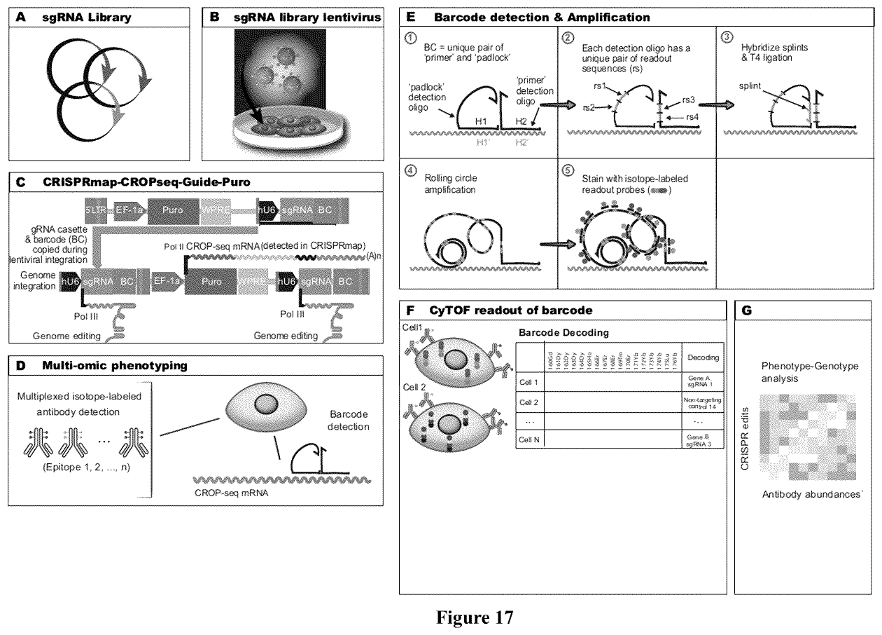

Pooled CRISPR screens typically introduce a single perturbation and its corresponding barcode in a cell through lentiviral infection at a low multiplicity of infection (FIG. 17A & FIG. 17B). Our barcode is expressed as part of an abundant mRNA encoding for a selection marker⁴ (FIG. 17C). In CRISPRmap, the cellular barcode consists of a unique combination of two adjacent 30 bp hybridization sequences. The first step of barcode detection occurs through hybridization of a pair of ssDNA oligos that are complementary to the adjacent hybridization sequences on the transcript¹⁸ (FIG. 17E). In our approach, the primer and padlock oligos each contain a unique pair of 20mer readout sequences. Collectively, the four 20mer sequences form a unique combinatorial readout set. Padlock probe circularization by T4 DNA ligase is dependent on hybridization of splint oligos, which bind to the 20mers on the primer oligo (FIG. 17E). Subsequently, rolling circle amplification is initiated through the primer oligo. Crucially, valid amplicons rely on AND-logic for the primer, padlock and both splint oligos. The readout set, and thus by extension the cellular barcode, is identified by cyclical hybridization rounds with dye conjugated oligos (readout probes)^{19,20}. Distributing the readout set over primer and padlock probe enables us to identify improperly self-ligated padlock oligos (readout set lacks primer readouts), or unallowed primer-padlock pairing (invalid readout set), and exclude them from analysis. Our cyclical hybridization readout approach was designed to minimize dependence on third party sequencing reagents, tissue degradation during cyclic enzymatic steps, and reagent cost of the assay (Table 1).

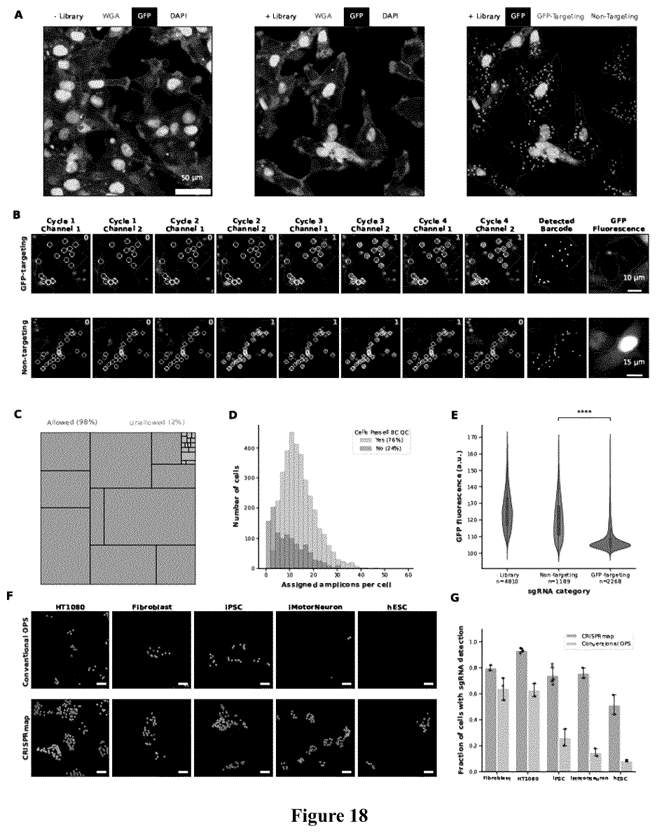

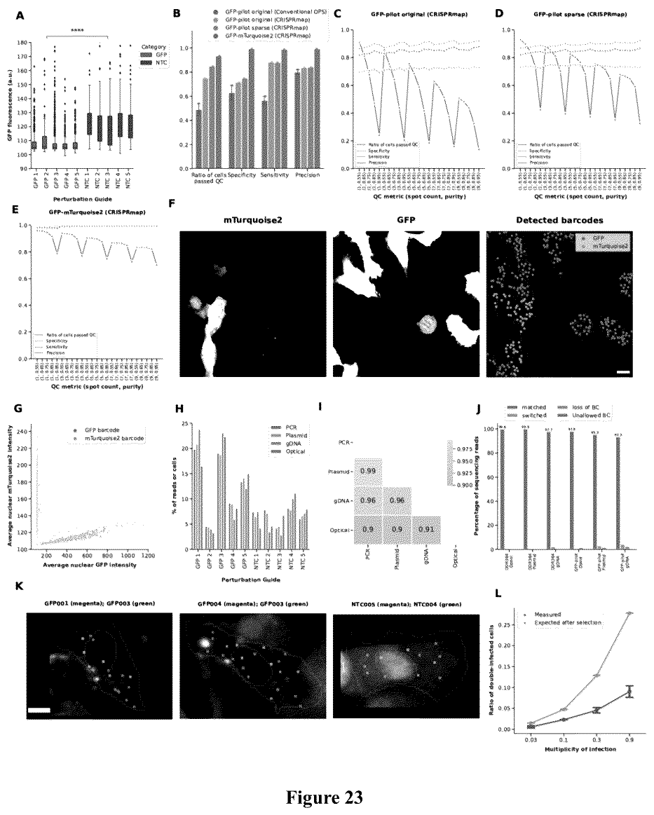

To develop and optimize our approach we transduced a small pilot lentiviral library containing five GFP targeting CRISPR guides, and five non-targeting CRISPR guides in a HT1080-Cas9 cell line expressing copGFP. We performed lentiviral library preparation (Methods) and infected cells at an MOI of <0.1 to ensure most infected cells will be edited by a single guide, and express a single barcode after puromycin selection. To couple our optical phenotype (GFP expression) to the CRISPR edit, we performed CRISPRmap (Methods, FIG. 18A). For this small pilot experiment, the readout set was imaged in 2 channels over 4 imaging cycles, larger libraries discussed later were imaged in 3 channels over 8 imaging cycles. Images across all barcode readout cycles and channels were co-registered into an image stack and corrected for global translational shifts (i.e. misaligned glass bottom well plate placement) as well as local translational shifts (i.e. cells slightly shifting between imaging rounds). To align the images across all imaging rounds we calculated the transformation matrices for each round using the TV-L1 implementation of optical flow²¹ on binary nuclei masks derived from DAPI stains (Methods). In our GFP targeting CRISPRmap screen barcode decoding is performed at amplicon level (Methods), by assigning an 8-bit code for each amplicon across the readout cycles and channels, where signal from each readout sequence yields a positive entry (1), and lack of signal a negative entry (0) (FIG. 18B). A guide identity (Guide ID) is assigned to an amplicon if the 8-bit code of the amplicon position matches a guide-identifying barcode in the pre-designed library codebook. We found that of all the amplicons that were positive for four readout probes, 98% coded for an allowed barcode included the library design, whereas 2% of amplicons reported an unallowed barcode (FIG. 18C), despite their relative ratios of 10/25 (40%) vs 15/25 (60%) possible primer-padlock pairs. In contrast, when we performed CRISPRmap readout on non-barcoded cells, we recover on average of 0.2 non-specific barcodes per cell, of which ˜62% stem from unallowed primer-padlock pairs, in agreement with their relative frequency (Table 2). Since CRISPRmap QC criteria require at least 3 barcode spots per cell with 2 out of 3 having the same barcode, unspecific binding is unlikely to affect precision of barcode assignment. From a per-cell analysis, we found that when imaging with a 20× objective, the median number of guide-assigned amplicons per cell was 11 (FIG. 18D). We restricted further analysis to cells with 3 or more amplicons and for which the most abundant barcode made up more than two thirds of the amplicons of the sum of the two most abundant barcodes under a cell segmentation mask. The latter criterion was put in place to retain cells for which imperfect cell segmentation could cover a few amplicons from neighboring cells, causing false association of guide-assigned amplicons to cell masks. With these quality control metrics in place, we retained 76% of the cells for further analysis (FIG. 18D, Table 2). Finally, we evaluated if we observed the expected optical phenotype for each of the guides in our pilot library and found indeed that cells with GFP-targeting guides have significantly lower GFP fluorescence levels than cells with NTC guides (FIG. 18E), which in turn have similar GFP fluorescence levels as unperturbed cells. Cells with each of the five GFP targeting guides showed significantly lower GFP levels than cells with any of the NTC control guides under pairwise comparison (FIG. 23A), thus recapitulating the expected genotype-phenotype relationship. In addition to the ratio of cells that passed QC, we evaluated sensitivity, specificity and precision (Methods) of CRISPRmap in this GFP pilot dataset, and found that they compare similarly or more favorably to conventional OPS⁴ (FIG. 23B). These metrics were relatively insensitive to our QC-metrics for calling a barcoded cell (FIGS. 23C-E). Most notable is the decrease in proportion of cells passing QC with increasing minimum number of barcoded amplicons, and increasing purity requirements. This effect was less pronounced when plating cells more sparsely (FIG. 23D & FIG. 23E), which we attribute to improved cell segmentation. To evaluate the effect of cell segmentation, we performed a ‘barnyard’ experiment where we mixed mTurquoise2 positive cells with GFP positive cells, each carrying a unique barcode. We observe excellent separation of the two populations and recover the expected barcodes (FIGS. 23F&G), and obtain high sensitivity, specificity, precision and proportion of cells assigned a barcode (FIG. 23B & FIG. 23E).

To assess guide representation throughout library preparation, infection and optical readout of the barcodes, we performed NGS sequencing on PCR product of the amplified synthesized DNA oligonucleotide pool, the CRISPRmap-CROPseq plasmid pool, and the genomic DNA from the cells transduced with the library. We further compared the sequencing result to the relative guide abundance of cells with optically identified guide identities, and observed highly correlated guide frequencies between all of these stages (FIGS. 23H&I). Moreover, analysis showed that there was minimal recombination that decoupled the sgRNA from its intended barcode during library prep. Sequencing of genomic DNA from infected cells revealed that 93% of the reads had a perfect match between sgRNA and CRISPRmap barcode (FIG. 23J), whereas 4% showed recombination such that a guide recombined with a different barcode in the pool, to which our optical readout is agnostic. Another 2% lost the barcode, and would thus not be detected optically. Very few reads either had no guide, or an unallowed barcode.

Interestingly, CRISPRmap barcode decoding at the amplicon level led to the detection of some double-transduced cells. These cells express two barcodes, each with a unique spatial pattern (FIG. 23K). Performing a series of experiments with increasing MOI indeed observed an increase of cells with double infections (FIG. 23L, Table 2), albeit a smaller increase than expected from Poisson statistics due to experimental conditions (Methods). This feature can be leveraged to study genetic interactions through combinatorial perturbations by infecting pooled libraries at a higher multiplicity of infection.

CRISPRmap Barcodes Primary, hESC, iPSC and Motor Neuron Cells

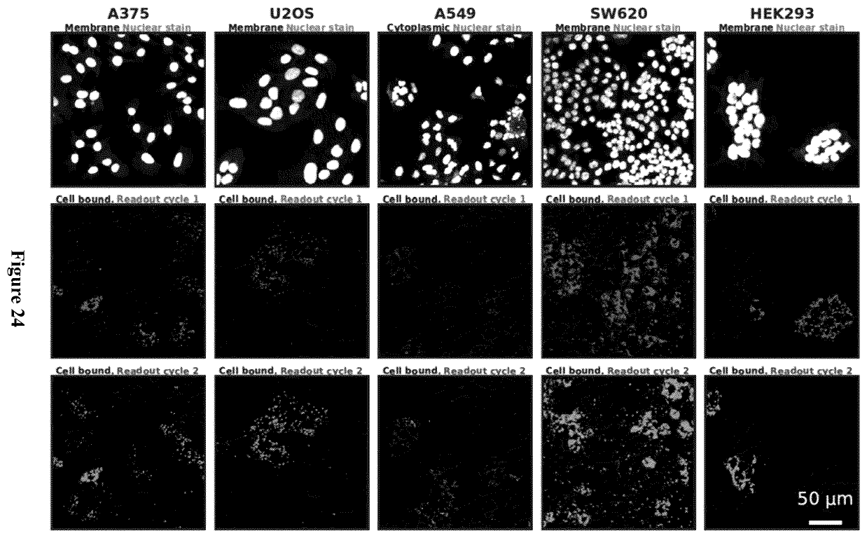

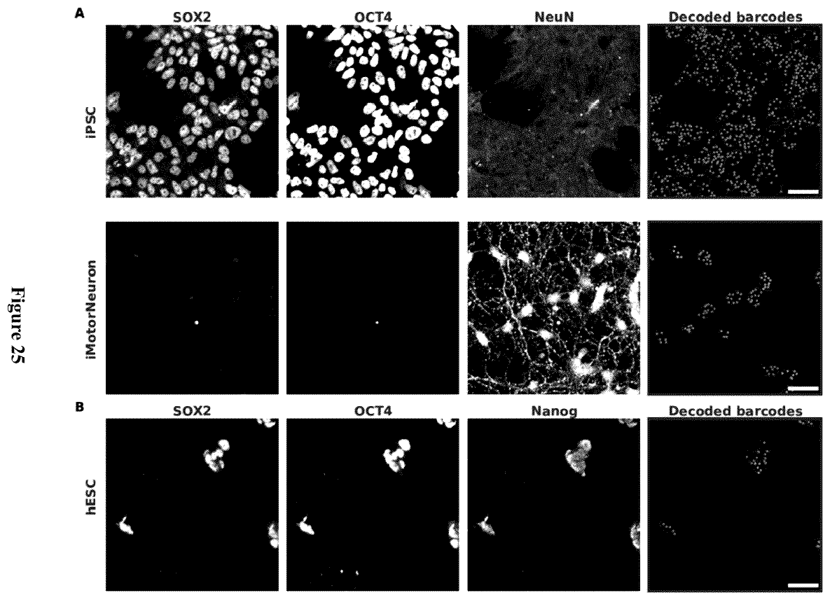

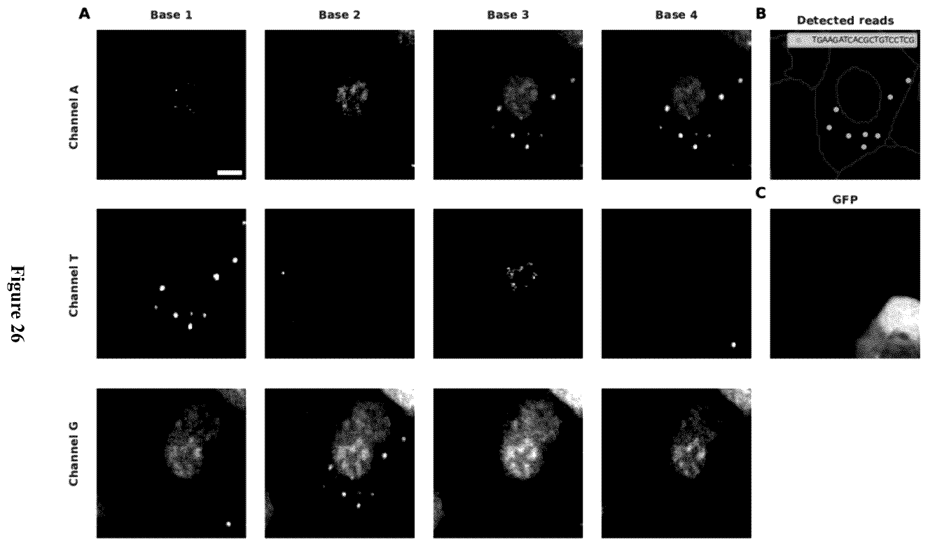

To evaluate CRISPRmap outside the context of immortalized or cancer cell lines (FIG. 24), we profiled primary fibroblast, hESC, iPSCs and motor neurons derived from iPSCs (iMN, FIG. 18F). We validated cell type marker (SOX2, OCT4 and NeuN) expression for hESC, iPSCs and iMN (FIG. 25). While conventional OPS performed well for HT1080 and fibroblasts (FIG. 18F & FIG. 18G, FIG. 26), CRISPRmap showed improved barcode detection in the more challenging cell types such as hESC, iPSCs and iMNs, recovering more barcode assigned amplicons per cell, and enabling a larger proportion of cells being assigned a barcode (FIG. 18F & FIG. 18G).

### Multi-Modal In Situ Profiling of Perturbed Cell States

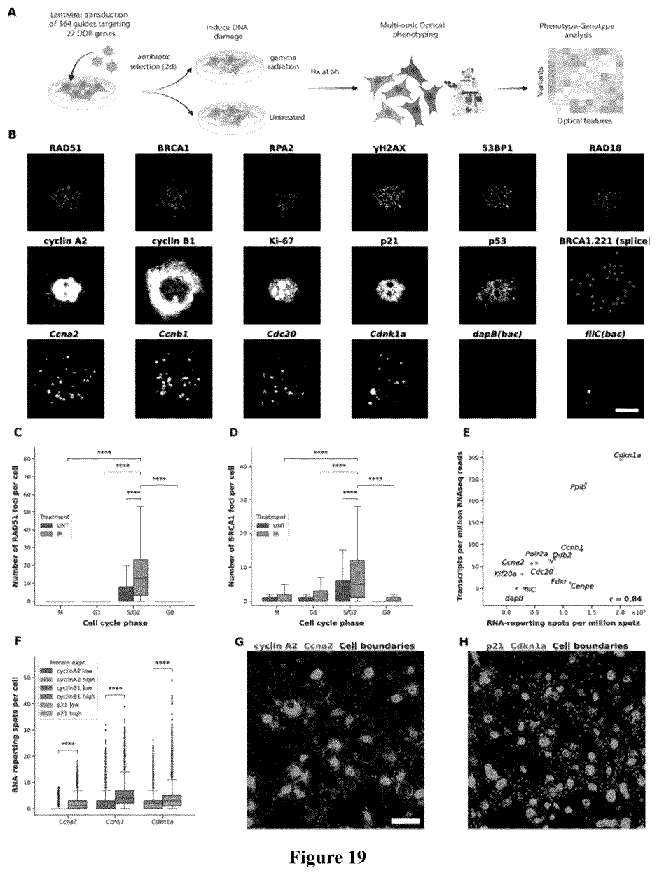

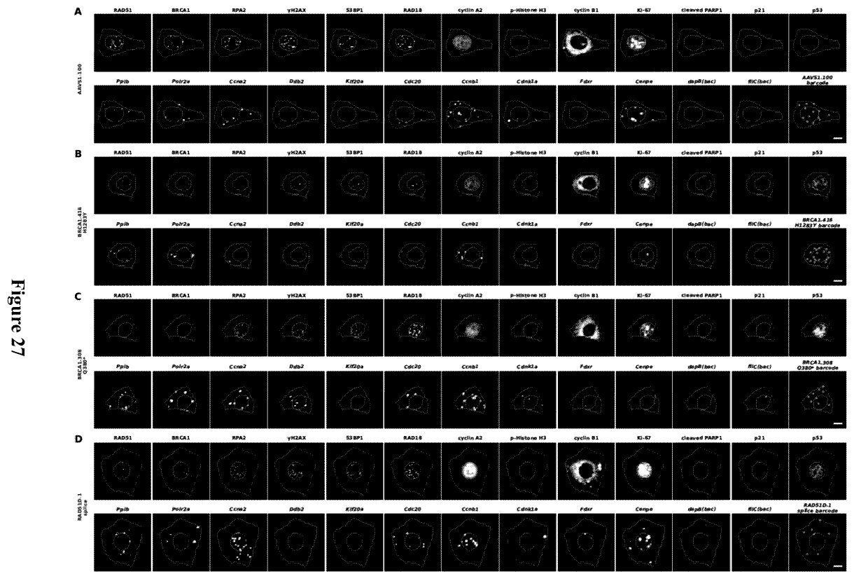

To unlock the potential of base editor scanning, we aimed to move beyond cellular fitness as a readout and enable detailed characterization of more complex biological processes. Therefore, we sought to combine base editing approaches with optical, single cell, multiplexed multi-modal approaches, measuring functional responses of dozens of proteins and mRNAs at subcellular resolution. We profiled the proteomic and transcriptomic responses of breast cancer cells to ionizing irradiation, a critical treatment modality for breast cancers, as a function of base-edited variants of 27 core DNA damage repair genes involved in the DNA damage response, homologous recombination and Fanconi anemia pathways. Specifically, a 364 sgRNA library was lentivirally transduced into an MCF7 cell line expressing BE3 (MCF7-BE3 hereafter)⁷. Transduced cells were selected in antibiotic-containing medium for two days and cultured in antibiotics-free medium for another two days, prior to induction of DNA damage by gamma radiation. Six hours after irradiation, cells were chemically fixed and profiled for protein expression, mRNA expression, and CRISPR barcode readout (FIG. 19A & FIG. 19B, FIG. 27). In this dataset, we profiled 226,369 single cells that met all barcode calling quality metrics, resulting in an average coverage of 310 cells per variant in the library in each experimental condition.

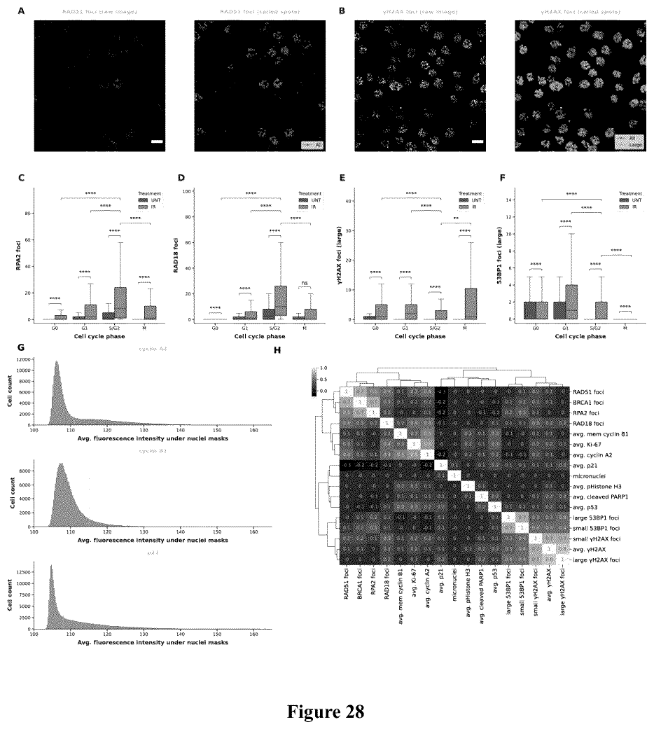

To evaluate how variants alter the cellular response following treatment with ionizing radiation, we applied a recently developed approach IBEX, which employs a cyclical process of antibody staining and chemical bleaching to facilitate high-resolution imaging of dozens of epitopes within a single sample, while preserving its physical integrity²². We visualized a panel of key DNA damage response proteins (RAD51, BRCA1, RPA2, γH2AX, 53BP1 and RAD18), cell cycle phase marker proteins (Ki-67, cyclin A2, cyclin B1, and phospho-Histone H3), and apoptosis-related proteins (cleaved PARP1, p21 and p53), and recovered expected subcellular protein localizations (FIG. 19B) and treatment-specific staining patterns (FIG. 19C & FIG. 19D). We also quantified the micronuclei formation based on DAPI stain (Methods). Accumulation of DNA damage response proteins at damaged genomic loci typically gives rise to punctate immunofluorescence detection patterns (foci) in the nuclei, which we quantify at the single-cell level through automated detection (Methods, FIGS. 28A&B). Cell cycle related proteins and transcription factors on the other hand are evaluated as average fluorescence across the cellular, nuclear and cytosolic mask (the latter we define as cell mask minus nuclear mask). Comparison of cytosolic to nuclear abundance of a protein allows for quantification of its translocation status. To further assess the specificity of the antibody staining, we compared the expression level and subcellular localization of the measured proteins in gamma-irradiated cells to untreated cells, while separating cells into four different cell cycle phases (G0-, G1-, S/G2, M-phase) based on the cell cycle markers we measured. As expected, we observe significant induction of nuclear foci formation for all the six measured DNA damage response proteins upon gamma irradiation (FIG. 19C & FIG. 19D, FIGS. 28C-F). In addition, we observe the expected significant enrichment of nuclear foci that function in the homologous recombination pathway (RAD51, BRCA1, RPA2 and RAD18) in the S/G2-phase. Large γH2AX foci are reported to be involved in double-strand break signaling²³ and 53BP1 foci believed to promote the non-homologous end joining pathway showed a slight enrichment in G1-phase over S/G2 phase (FIGS. 28E&F). Finally, the single-cell and highly multiplexed nature of our data enables us to evaluate the correlation among all optical features we measured (FIG. 28H). As expected, we observe a positive correlation between the nuclear foci involved in homologous recombination (RAD51, BRCA1, RPA2 and RAD18) with the average nuclear intensity of the S/G2-phase markers (cyclin A2, cyclin B1) and the proliferation marker (Ki-67).

To simultaneously measure the transcriptomic response of cells, we adapted our CRISPRmap barcode detection approach to detect endogenous mRNA transcripts, and call this approach RNAmap (FIG. 17C), which differs from CRISPRmap in three key ways. First, the transcript hybridizing regions of the primer and padlock detection oligos target adjacent sequences on the endogenous RNA transcripts. The design of gene-specific detection oligos was refined for specificity, a narrow range of melting temperatures for primer and padlock oligos, minimal off-target binding and secondary structure (Methods). Secondly, to promote detection efficiency, we increased the number of primer-padlock oligo pairs to six per RNA transcript. Primer-padlock pairs that share an RNA target also share the same set of readout sequences. Thirdly, to further boost detection efficiency, RNAmap primer detection oligos only encode a single readout sequence, and as a result, only a single splint oligo needs to undergo ligation to form an RCA template. Consequently, the padlock oligo encodes three readout sequences to enable similar combinatorial readout of transcript identities.

We applied RNAmap to target a panel of 12 genes, selected for their expression in irradiated MCF7-BE3 cells, and to span a range of expression levels. The transcripts profiled include cell cycle-related (Ccnb1, Ccna2, Cdkn1a, Cdc20, Kif20a, Cenpe), housekeeping (Ppib & Polr2a), DNA damage response-related (Ddb2, Fdxr) and bacterial negative control (dapB, fliC) genes (FIG. 19B). We validate the specificity of detection by comparing optically identified transcript-reporting spots at the population level to bulk RNA-sequencing reads, and observe a Pearson correlation of 0.84 (FIG. 19E). Additional support of specificity is provided by the analysis of the correlation between mRNA and protein expression level for the three cell-cycle related genes. Cells for which we observe high abundance (FIG. 28G) of cyclin A2, cyclin B1, and p21 at the protein level, have significantly (p<0.0001, two-sided Mann-Whitney U test) more corresponding transcripts detected by RNAmap (FIG. 19F, FIG. 19G, FIG. 19H).

### Variant Screen Recapitulates DNA Damage Response Mechanisms

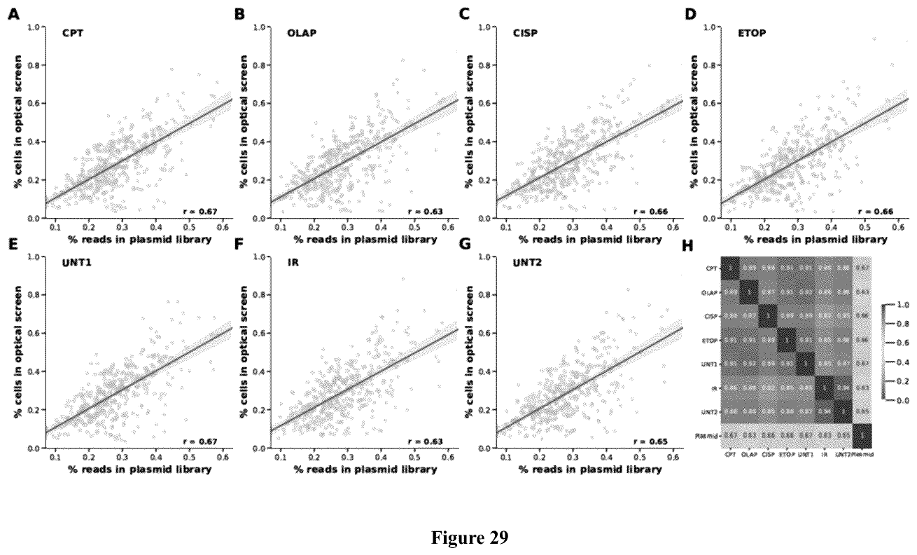

Building upon our prior work assessing human nucleotide variants across 86 DDR genes and assessed their effect on cell viability⁷, we selected variants that significantly altered viability in at least one treatment condition in the prior study, we focused the present study on sgRNAs with a single C base in their editing window, to minimize confounding effect of bystander mutations that could obfuscate the phenotypic consequences associated with a guide. Combining the 292 guides targeting DDR genes with 35 guides targeting the AAVS1 safe-harbor site and 37 non-targeting control (NTC) guides that have minimal targets in the human genome, we applied a 364-guide library (referred as DDR364) to the MCF7-BE3 cells for a multi-modal pooled optical base-editing screen. Our library includes 162 missense guides, 50 nonsense guides and 80 splice guides (variants that affect splice-donor or -acceptor sequences). It is expected that nonsense variants and splice variants are more detrimental to protein function than the missense variants, which are associated with a broader range of effects on protein function. Our library includes 64 variants that the ClinVar database²⁴ annotates as pathogenic/likely pathogenic (P/LP) variants, and 75 variants with uncertain significance (VUS). Guide representation of the DDR364 library as sequenced in the plasmid library, and recovered by optical barcodes is listed in Table 3, and the correlation between guide representation and plasmid library sequencing reads is shown in FIG. 29.

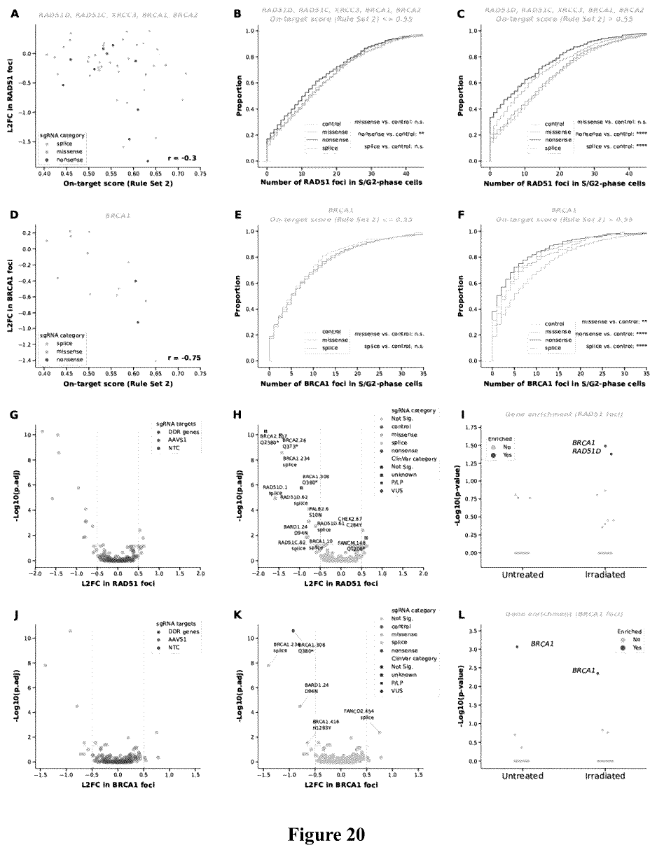

We set out to validate if CRISPRmap could recapitulate known phenotypes as a function of specific base edits. First, similar to our previous fitness study⁷, we found stronger phenotypic changes for guides with higher Rule Set 2 (RS2) on-target efficiency scores, initially created for CRISPR-KO sgRNAs²⁵. Specifically, we first evaluated the abundance in RAD51 foci for variants of genes that are essential for RAD51 foci formation (RAD51D, RAD51C, XRCC3, BRCA1, BRCA2) relative to negative control guides. Here, we observe a trend of decreasing RAD51 foci with guides with higher RS2 scores, especially for guides that are from the deleterious (nonsense and splice) categories (FIG. 20A). Notably, implementing a minimum Rule Set 2 score threshold significantly improved the differentiation between deleterious and negative control guides (two-sided KS-test, p.adj<0.0001, FIG. 20C), while splice variants below the RS2 threshold show no statistical significance over control guides (two-sided KS-test, p.adj>=0.05, FIG. 20B). Missense guides targeting these genes generally show much milder impact on the abundance in RAD51 foci. Similarly, phenotypic changes in BRCA1 foci abundance of BRCA1 variants strongly correlated with RS2 scores (Pearson r=−0.75, FIG. 20D), and cells expressing nonsense or splicing variant guides with RS2>=0.55 showed significantly fewer BRCA1 foci than cells expressing negative control guides (FIG. 20F). This distinction was not significant for guides with lower RS2 scores (FIG. 20E).

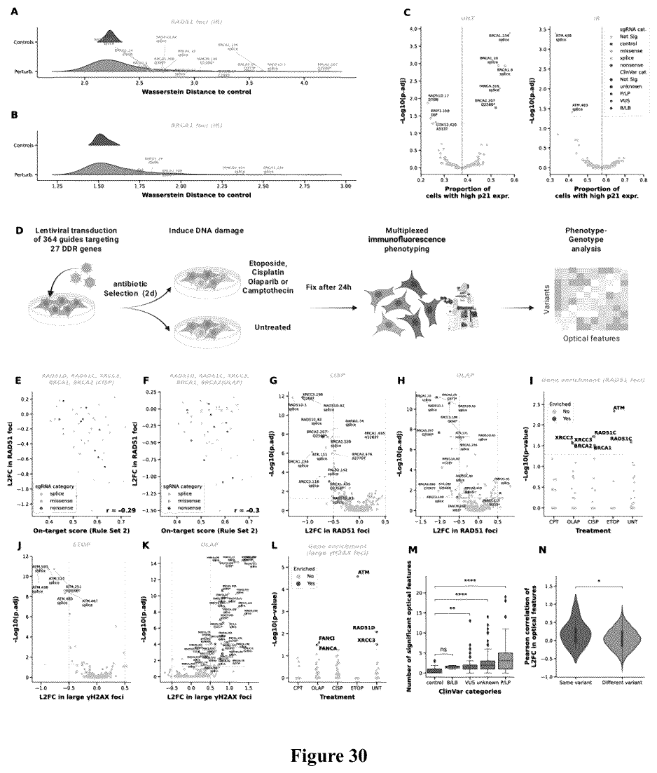

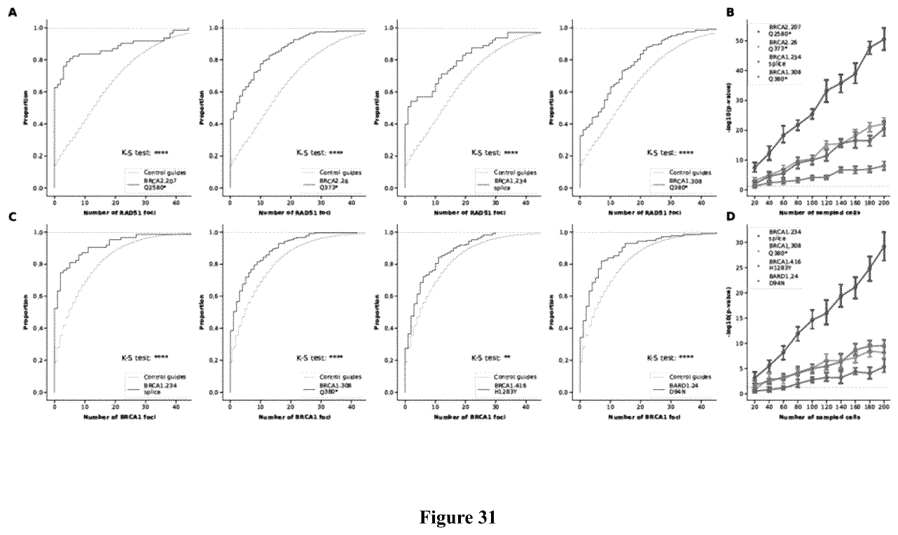

Secondly, we considered variants to significantly alter nuclear foci formation if there was an absolute log 2-fold change (L2FC) >0.5 in the mean number of foci per cell when compared to the population of negative control guides, and we observed a Benjamini-Hochberg corrected two-sided KS-test p-value (p.adj)<0.05. Based on these criteria, we observe that none of the negative control guides (AAVS1-targeting or NTC guides) significantly alter the number of RAD51 or BRCA1 foci (FIG. 20G & FIG. 20J). Most of the hits significantly reducing the number of RAD51 foci are nonsense or splice variants, annotated in ClinVar as pathogenic or likely pathogenic (P/LP), but we also identify two missense variants that are annotated as variants with uncertain significance (VUS) or not documented (unknown) in the ClinVar database (FIG. 20H). Furthermore, guides that target the BRCA1 and RAD51D genes are significantly enriched in the guides that lead to significant changes in RAD51 foci in the irradiated cells (FIG. 20I), whereas variants that are identified to significantly change the BRCA1 foci (FIG. 20K) are enriched for BRCA1 variants (FIG. 20L), as expected. A BRCA1 missense VUS, and an unknown BARD1 missense variant were identified to significantly reduce BRCA1 foci in irradiated cells. The hits identified based on L2FC and p.adj thresholds were further confirmed by the measurement of bootstrapped Wasserstein distance (Methods) of each guide from the cells with control guides for RAD51 foci (FIG. 30A) and BRCA1 foci (FIG. 30B). Analysis of the top 4 variant hits for RAD51 and BRCA1 (FIG. 31A & FIG. 31C, respectively) showed that on average those hits would have been distinguished as significant upon screening ˜60 cells per guide (FIG. 31B & FIG. 31D, Methods).

Apart from nuclear foci, we also analyzed protein stains such as cyclin A2, Ki-67 and p21, for which cells can be categorized into high or low protein expression categories based on the average nuclear fluorescence of the protein stain. Instead of L2FC analysis, for these stains we performed beta binomial testing (Methods) and observe that three BRCA1 splice variants significantly upregulated the proportion of cells with high p21 expression in the untreated cells (FIG. 30C, left) whereas two ATM splice variants reduced the proportion of cells with high p21 expression in irradiated cells (FIG. 30C, right).

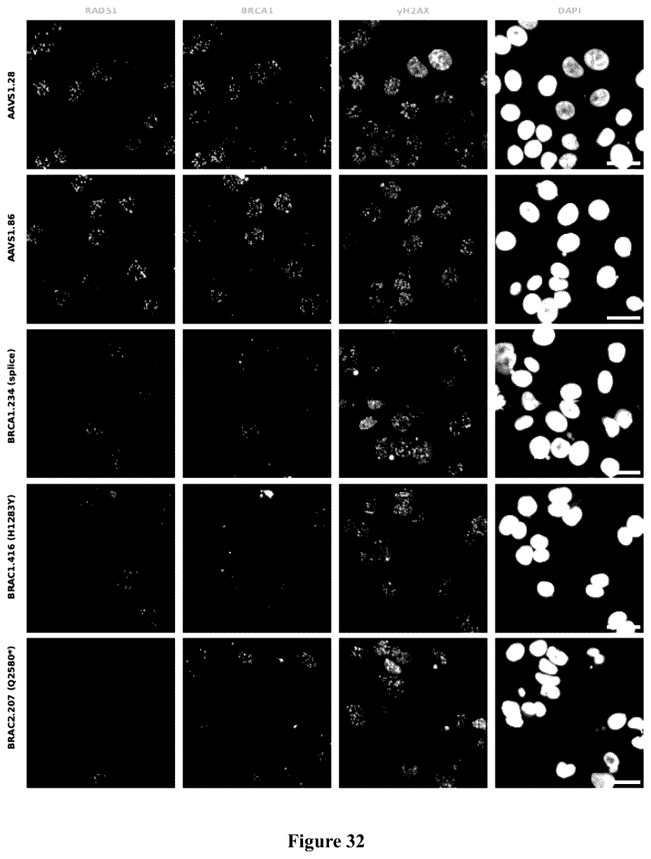

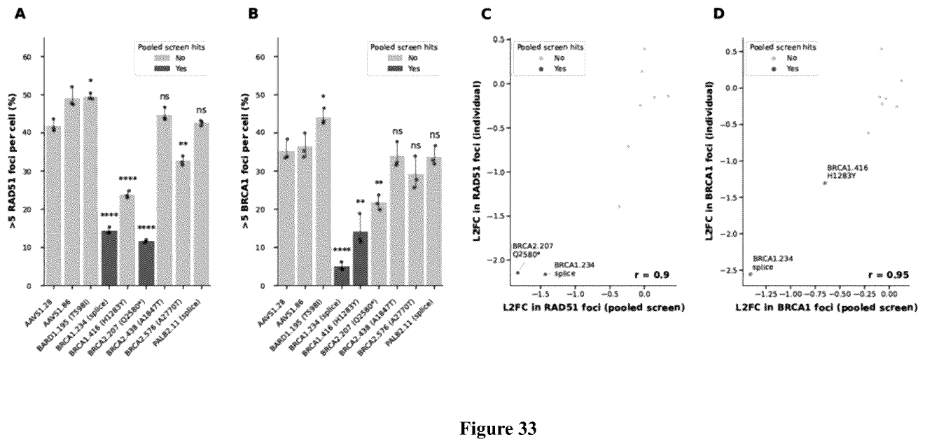

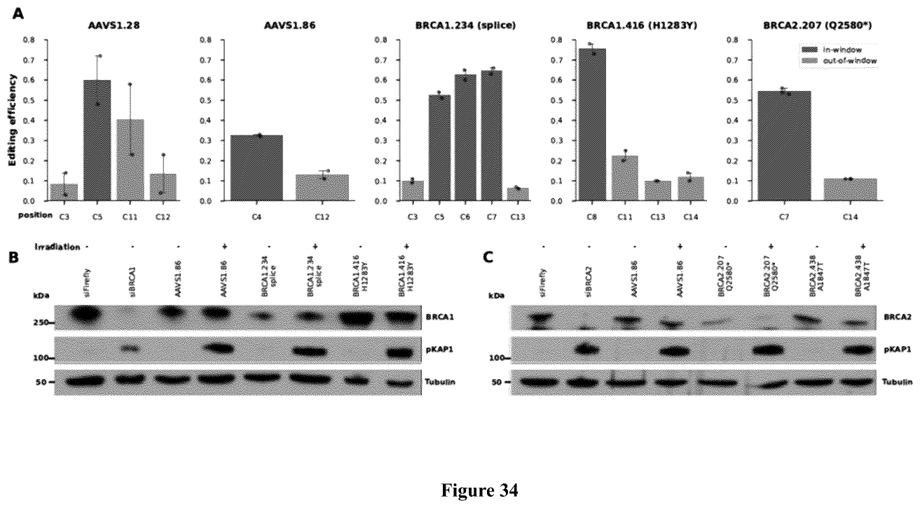

To further verify perturbation-specific phenotypic changes observed in the pooled screens, we selected 2 AAVS1-targeting control guides and 7 guides targeting BRCA1, BRCA2, BARD1, and PALB2 from different sgRNA and ClinVar categories and with on-target score (RS2) >0.5 (Table 3). BRCA1.234 (splice, P/LP) and BRCA2.207 (Q2580*) (nonsense, P/LP) were identified as hits for RAD51 foci reduction in the pooled screen, whereas BRCA1.234 (splice, P/LP) and BRCA1.416 (H1283Y) (missense, VUS) as hits for BRCA1 foci reduction. We transduced MCF7-BE3 cells with each guide individually, and measured the L2FC in RAD51 and BRCA1 foci compared to all control cells in the gamma-irradiated cells. FIG. 32 shows representative images for RAD51, BRCA1 and γH2AX foci. We quantified the percentage of cells with >5 RAD51 and BRCA1 foci (FIG. 33A & FIG. 33B, respectively) and observed that screen hits transduced as individual guides resulted in a significantly lower percentage, compared to the cells transduced with AAVS1-targeting guides, whereas non-screen hits generally showed no or low significance. High correlations of L2FC are observed between guides transduced in the pooled library and transduced individually (Pearson r=0.90 for RAD51 foci, FIG. 33C; Pearson r=0.95 for BRCA1 foci, FIG. 33D), with hits identified in the pooled screening also showing the most significant changes when transduced individually. In addition, we characterized the base-editing efficiency of the three hits we identified in the pooled screen (BRCA2.207 (Q2580*), BRCA1.234 (splice), BRCA1.416 (H1283Y)), together with two AAVS1 controls (AAVS1.28, AAVS1.86). Sanger sequencing of PCR-amplified genomic DNA around the intended editing region for all three hits revealed a high in-window C base editing efficiency (between 50% and 75%) whereas out-of-window C bases typically showed an editing efficiency lower than 25% (FIG. 34A).

We also applied the same DDR364 library on MCF7-BE3 treated with DNA damaging agents (Camptothecin, CPT; Olaparib, OLAP; Cisplatin, CISP; Etoposide, ETOP) to study the treatment-specific responses of the gene variants (FIG. 30D) with a primary focus on the formation of RAD51 and large γH2AX foci. Similar to the ionizing irradiation screen, we observed that guides targeting the RAD51-relevant genes show more significant loss in RAD51 foci with a higher RS2 score, especially in cells treated with CISP and OLAP (FIGS. 30E&F). A RS2 score threshold of 0.5 distinguishes deleterious guides that show mild and strong phenotypes. Again, we observe most of the hits reducing the RAD51 foci in CISP- and OLAP-treated cells are splice or nonsense variants, many of which being P/LP (FIGS. 30G&H), and a significant enrichment of guides targeting RAD51 paralog genes, BRCA1, or BRCA2 can be seen (FIG. 30I). A drastic difference in the hits can be seen when comparing the change in large γH2AX foci in ETOP-treated (FIG. 30J) and OLAP-treated (FIG. 30K) cells. In ETOP-treated cells, we observe most splice variants of ATM significantly reduced the number of large γH2AX foci, whereas many deleterious variants coming from a mixed background of RAD51-related genes, ATR, FA pathway genes, increased large γH2AX foci under other treatments. The enrichment test shows a significant enrichment of FANCA- and FANCI-targeting guides in OLAP-treated cells and ATM-targeting guides in ETOP-treated cells among hits of large γH2AX foci (FIG. 30L).

Collectively, these results underscore that CRISPRmap effectively couples barcodes to their corresponding guides, and that the expected phenotypic changes are detected by profiling a few hundred cells per variant, despite imperfect efficiencies associated with current base editors. Moreover, CRISPRmap enables the dissection of therapeutic treatment-specific responses of known pathogenic variants and the identification of variants with significant phenotypic effects in the DDR pathways.

Optical Screening Correlates VUS with Pathogenic Variants

Interpreting the functional implications of somatic mutations in cancer, primarily characterized by single nucleotide changes that often lead to missense VUS variants remains a challenging endeavor. This challenge poses a barrier to effective diagnosis, patient stratification, and the management of drug-resistant diseases. Utilizing experimental approaches is crucial in assessing the functional impact of VUS. This is essential for establishing a link between VUS and disease-related phenotypes, particularly due to the limited availability of clinical datasets, and the infrequent occurrence of certain variants in patient cohorts.

We set out to chart variant effects on the DDR pathways by combining the two CRISPRmap base-editing screens that treated cells with ionizing radiation or 4 DNA damaging agents commonly used for cancer therapy. In total, we profiled 948,604 cells that passed barcode QC metrics, averaging 372 cells per guide in each treatment condition. We compared benign, VUS, unknown and pathogenic/pathogenic like variants with our negative control guides, and found that only benign variants were not significantly different from controls when evaluated on the number of foci features with statistically significant changes from the control cells across all treatment conditions (FIG. 30M), whereas P/LP variants led to the highest number of features with significant changes. Moreover, when evaluating the correlation between the L2FC values of optical features between guide pairs, we observed that the distribution of correlations between pairs of sgRNAs that are designed to induce the same variant is higher than the distribution of correlations between all pairwise sgRNAs targeting a particular gene (FIG. 30N).

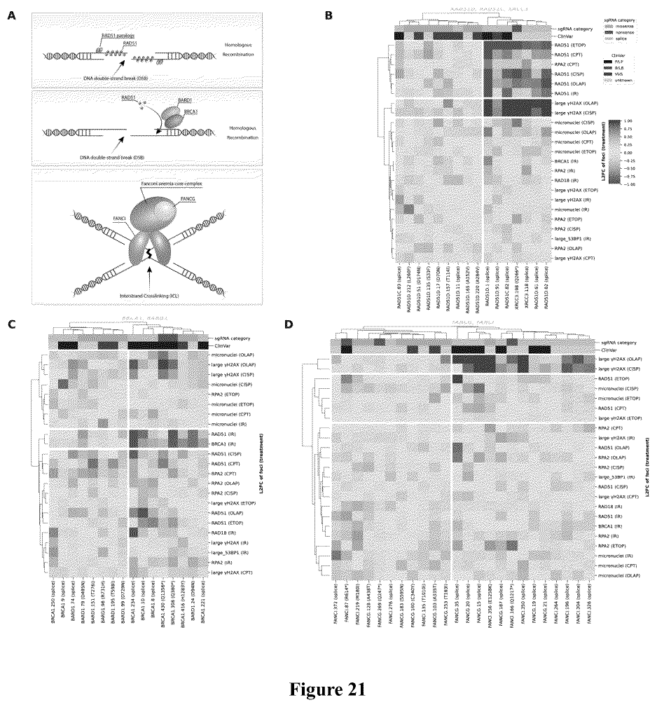

We subsequently clustered variants from functionally related genes based on the optical features measured in the ionizing irradiation (IR) and DNA damaging agents (CPT, OLAP, CISP, ETOP)-treated cells. When we clustered the variants from the RAD51 paralog genes (RAD51C, RAD51D and XRCC3), we observed a cluster composed of most splice and nonsense variants (FIG. 21B, right cluster). This cluster showed a reduction in RAD51 foci across all treatment conditions and a strong upregulation of γH2AX foci for OLAP and CISP treatments, whereas most missense variants form a separate cluster with more mild phenotypic changes (FIG. 21B, left cluster).

Variants of BRCA1 and its heterodimeric binding partner BARD1 can also be categorized into two clusters. The right cluster contains most splice and nonsense P/LP variants and shows an expected reduction in RAD51 and BRCA1 foci upon irradiation, as well as an increase in large γH2AX foci and micronuclei mostly in OLAP and CISP-treated cells (FIG. 21C). Variants in the left cluster are mostly missense mutations with milder phenotypes. Notably, a missense VUS variant of BRCA1 (BRCA1.416 (H1283Y)), that renders the H1283Y amino acid change on the BRCA1 protein, clusters with pathogenic variants of BRCA1. Despite being classified as VUS, a recent study²⁶ classified it to be likely pathogenic based on a BE3 base-editing screen with fitness as a readout, and they confirmed the loss in cell viability for H1283Y with CRISPR-mediated homology-directed repair. To assess if the missense BRCA1 VUS variant had a phenotype similar to a nonsense mutation due to a change in protein stability, we performed immunoblotting on cells transduced with individual guides, and found the missense variant to have full length protein at similar abundances as the AAVS1 control variant (AAVS1.86), whereas the splice variant (BRCA1.234 (splice)) shows a reduction in full-length protein similar to the siRNA-induced BRCA (siBRCA1) knockdown control (FIG. 34B). We also performed immunoblotting on two BRCA2 variants to further investigate the protein stability of different types of variants and we observe a loss of full-length BRCA2 protein in the nonsense variant (BRCA2.207 (Q2580*)) similar to the siRNA-induced BRCA2 (siBRCA2) knockdown control, whereas the missense VUS variant (BRCA2.438 (A1847T)) has similar full-length protein to the AAVS1 control variant (AAVS1.86) (FIG. 34C). Immunoblotting on phospho-KAP1 (pKAP1) indicates the induction of DNA damage upon gamma irradiation and we observe no correlation between the changes in protein stability and the DNA damage (FIG. 34C).

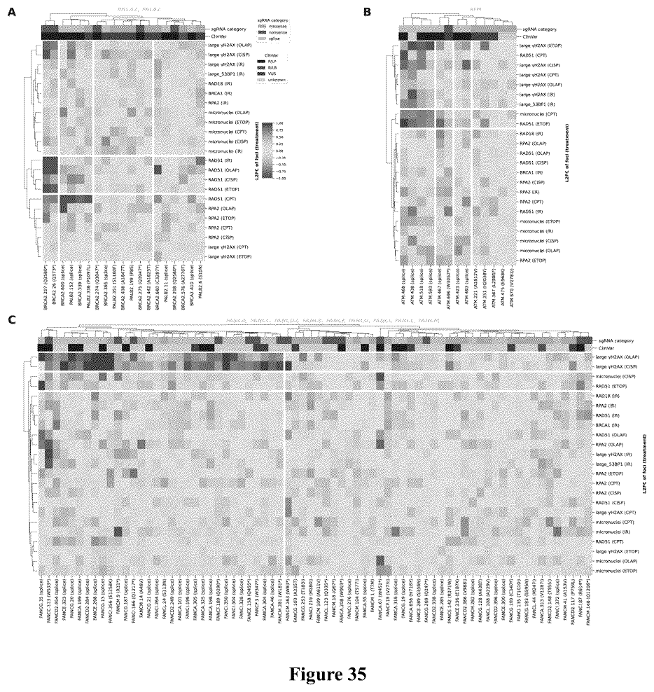

Evaluating variants of Fanconi anemia complementation group (FANC) members FANCI and FANCG reveals a cluster (FIG. 21D, right) that predominantly consists of splice and nonsense variants, with the exception of a single missense FANCI variant (FANCI.356 (E1258K)) that increases γH2AX foci for OLAP and CISP treatments far more strongly than other FANC missense variants. Clustering of all FANC gene variants also identifies clusters with one cluster containing most splice and nonsense variants, showing a similar optical feature signature as in the FANCI-FANCG clustering result (FIG. 35C). Besides the missense variants of FANCI, two additional missense variants of FANCM (FANCM.14 (A46V)) and FANCL (FANCL.24 (S113N)) were observed with a similar optical feature signature. Variant clusters with notable signatures can also be found in the clustering outcomes of other functionally related genes, such as BRCA2 and PALB2 (FIG. 35A) and ATM (FIG. 35B).

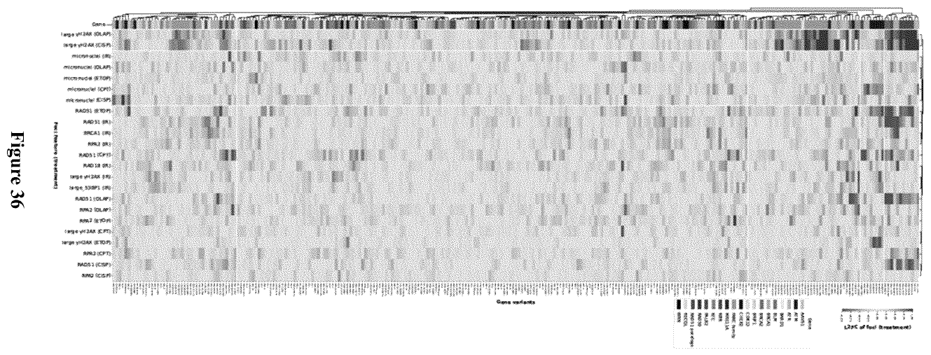

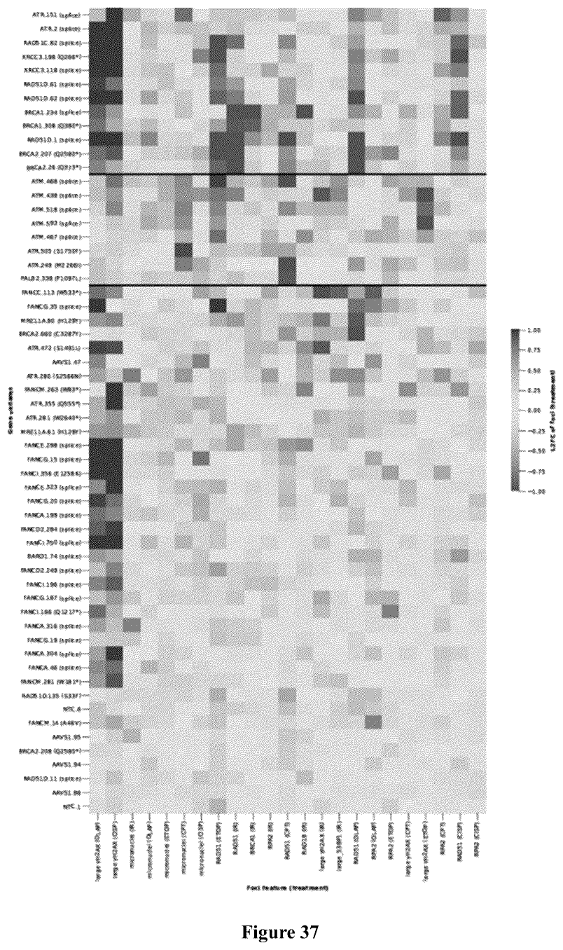

Moreover, we performed hierarchical clustering on the 273 guides in the library that have a Rule Set 2 on-target score >=0.5 (FIG. 36). A zoomed-in version of the 3 top clusters (FIG. 37) shows a significant enrichment in RAD51 paralogs (RAD51D, RAD51C, XRCC3) variants in the top cluster (p=1.1e−5, hypergeometric test), featured by downregulation of RAD51 foci across all treatments and upregulation of γH2AX foci in CISP- and OLAP-treated cells. We also observed that ATR, BRCA1, and BRCA2 variants clustered together with these RAD51 paralog variants, showing similar patterns of phenotypic changes, suggesting a disrupted homologous recombination response to DNA double-stranded breaks. Besides, strong enrichment of ATM variants is observed by the second top cluster (p=5.4e−6, hypergeometric test), where γH2AX foci are significantly reduced in ETOP-treated cells and mostly down-regulated in CISP- and OLAP-treated cells. FANC family gene variants are observed to be enriched in the third top cluster (p=3.2e−5, hypergeometric test) which features an upregulation of γH2AX foci in OLAP- and CISP-treated cells.

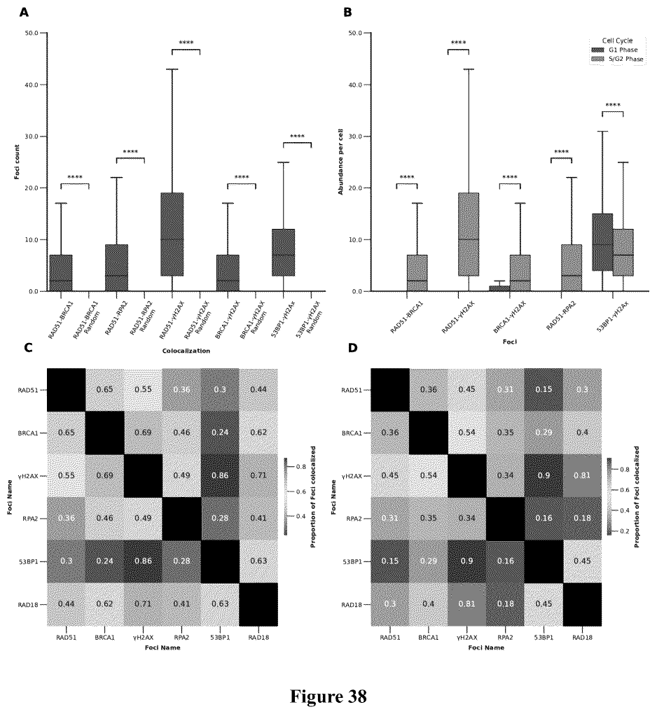

Beyond the investigation of phenotype change in each type of foci, we performed colocalization analysis on the 6 DNA damage response foci (RAD51, BRCA1, RPA2, γH2AX, 53BP1, RAD18) (Methods) to investigate the foci-to-foci colocalization as a result of perturbations. We performed this analysis on the ionizing radiation (IR) dataset, as our main focus was on BRCA1-RAD51 and BRCA1 or RAD51 colocalizations with other foci during the process of homologous recombination. To confirm that the foci colocalize due to biological reasons and not by random chance, we simulated the chance of random colocalization within the nucleus and observed that foci colocalizations are not random (p<0.0001, Methods, FIG. 38A). We then compared the differences in abundance of foci pair colocalizations between G1 and S/G2 phase, and observed an increase in BRCA-RAD51 colocalization in the S/G2 phase as expected (FIG. 38B). We also calculated the proportion of individual foci colocalizing with another foci, and as expected, we observed a high proportion of RAD51-BRCA1 foci colocalization in S/G2 phase, whereas high proportions were observed between 53BP1-γH2AX foci in both G1 and S/G2 phases (FIGS. 38C&D). These results highlight the possibility of investigating higher order interactions between different proteins involved in DNA damage response.

As a whole, these data support that CRISPRmap not only empowers the analysis of missense VUS variants by their correlation with known pathogenic splice or nonsense variants on functionally related genes but also characterizes the drugs-specific responses of these gene variants for multiple DDR pathway regulators. The high-multiplexed nature of CRISPRmap phenotyping further identifies unique optical signatures of variant clusters that sheds light on the molecular mechanisms of known pathogenic variants as well as their correlated VUS variants, making CRISPRmap a potential tool to advance patient-specific precision medicine strategies.

CRISPRmap Couples Barcode Detection with Cyclic IF in Tissue

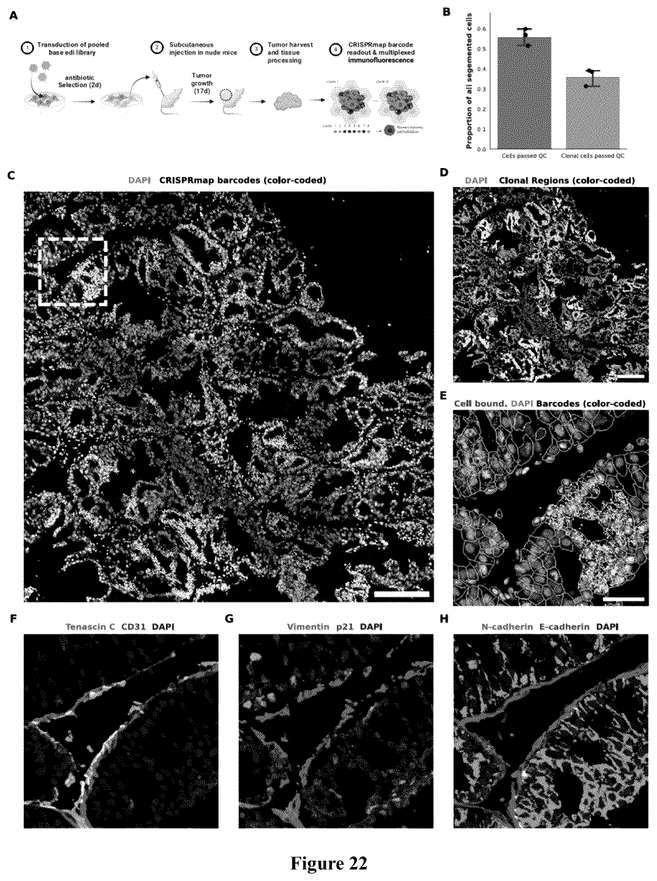

To evaluate if we could read CRISPRmap barcodes in a tissue context, we performed a pilot study and transduced Cas9 negative OE19 (human oesophageal carcinoma) cells with the aforementioned DDR lentiviral library. Following antibiotic selection and expansion, 5 million cells were suspended in a 1:1 mixture of matrigel and PBS and inoculated into the flanks of a nude mouse. Tumor tissue was harvested after 17 days and flash frozen (FIG. 22A). The CRISPRmap protocol was performed on sectioned tissue, with minor modifications (Methods) to yield ample barcode detection throughout the section (FIG. 22C) in recognizable epithelial growth patterns, suggesting that single cells yielded local clonal outgrowth. Subsequent multiplexed immunofluorescence profiling enabled cell and nucleus segmentation based on E-cadherin (FIG. 22E) and DAPI stains respectively. About 76 percent of the nuclei in the tissue sections were contained in cell segmentation mask obtained from E-cadherin stains, indicating that about one fourth of the tumor cells are non-cancer cells. Evaluation of barcode signal for segmented cells revealed that 56% of segmented cells pass our barcode QC metrics (FIG. 22B), and the median number of barcodes detected per cell is 14 (Table 4). We expect that the lower number of cells with barcode assignment is due to a variety of reasons, including a lack of antibiotic selective pressure during 17 days of tumor growth, enabling cells to silence the barcode expression. Furthermore, imperfect cell segmentation of the morphologically diverse cancer cells can complicate barcode purity, and segment small non-cancer cells by mistake. Analysis revealed that cells with mask size <250 pixels show a significantly lower count of barcode assigned amplicons (p=9.3e−56, two-sided Mann-Whitney test) with a median of 0.0 spot and a mean of 2.2 spots, compared to the rest of the cells with a median of 5.0 spots and mean of 12.4 spots (Table 4). These small cells contribute to 7.1% of the cells we segmented and analyzed. Another mechanism that leads to poor barcode readout is cell death. We performed an analysis based on the average fluorescence intensity of the cleaved PARP staining under cell nuclei masks and classified 3.6% cells as cPARP+. We observed a significantly lower spot count (p=4.0e−18, two-sided Mann-Whitney test) with a median of 0.0 spot count for the most represented barcode in cPARP+ cells, compared to a median of 5.0 spots in cPARP− cells (Table 4).

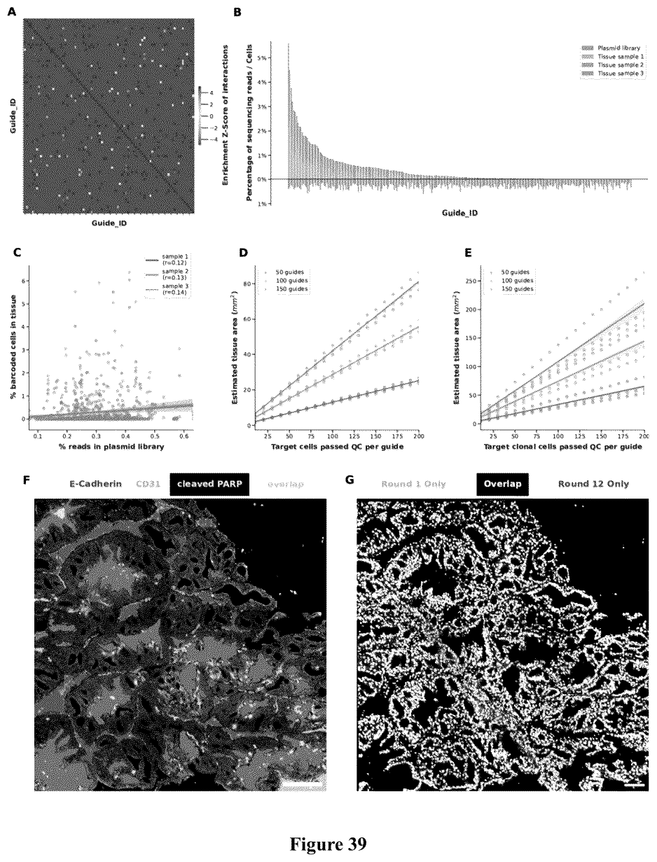

In addition, we performed clonality analysis²⁷ of barcoded cells in a cell-centric manner based on 10 nearest neighbors graphs (Methods). Cells with significant enrichment for their barcode in the 10 nearest neighbors, along with the same guide cells in the neighbor graph, were plotted to identify clonal regions (FIG. 22D). This analysis revealed that 36% of E-cad segmented cells (or 64% of barcoded cells) are considered part of a clonal region (FIG. 22B). Analysis of interaction patterns between clones¹⁷ shows that subcutaneous OE19 tumors have a predominantly clonal distribution (FIG. 39A) when compared to other reported tumor types.

We observed a strong skew of the library of guides detected across the 3 different tissue sections evaluated (FIGS. 39B&C). Out of the 364 guides present in the DDR library, we observe 192 guides with at least 3 cells across the tissue sections evaluated, of which 133 were present in clonal regions. Based on literature, we expect the degree of library skew to be cancer/model dependent, as such, we provide an estimate of the tumor tissue area needed to profile as a function of the number of barcoded cell per guide, and assuming the relative frequency in the tissue is that of the one observed in the plasmid library (FIG. 39D&E).

Further antibody staining cycles allowed for the visualization of angiogenesis (CD31, endothelial marker, FIG. 22F), extracellular matrix formation (Tenascin C, FIG. 22F) around tumor domains, as well as a layer of cells expressing vimentin (FIG. 22G) and N-cadherin (FIG. 22H), and transcription factor nuclear translocation in the transplanted cells (p21, FIG. 22G). We observed areas in the tumor tissue without cells, and wondered if this is due to cell loss during tissue preparation. We evaluated the loss of nuclei between the first cycle of imaging (barcode readout), and the last round of imaging (antibody stain), and observed that 95.6% of nuclei can be registered between these rounds (FIG. 39G), indicating we don't observe significant loss during barcode and antibody readout rounds. Next, we noticed that cells facing the areas without cells, which we termed ‘voids’, tend to be positive for either CD31 or cPARP staining. Voids were annotated (Methods) as vasculature if cells around the void were enriched for CD31 staining, and as necrotic areas if enriched for cPARP staining (FIG. 39F).

Iterative immunofluorescence and optically resolved transcriptomics approaches have been established for comprehensive profiling of intracellular, cellular, extracellular and signaling mechanisms in the tumor microenvironment. Coupling spatial genomics to CRISPRmap thus enables in vivo CRISPR screens at subcellular resolutions to systematically interrogate how genomic alterations and protein function modify cellular behavior in a tissue context and/or effects upon the cellular microenvironment. Moreover, CRISPRmap is a CRISPR enzyme agnostic barcode readout, and thus adaptable to the wider CRISPR toolkit, including base editing, gene activation and epigenetic modifications, enhancing the potential to uncover the influence of genes and their regulatory mechanisms on cellular organization and microenvironment.

### Discussion

Our study introduces CRISPRmap, a sequencing-free in situ CRISPR barcode readout approach coupled with cyclic immunofluorescence and in situ RNA detection, CRISPRmap expands upon traditional boundaries of optical pooled genetic screening. The AND logic used by CRISPRmap is designed to increase detection specificity when compared to single oligo detection approaches since it requires the adjacent hybridization of both the primer and padlock detection oligos. For approaches that only require a single detection probe that carries the full barcode, and don't require a gap-fill reaction, any non-specific oligo could give rise to an amplicon if the 5p and 3p ends of the detection oligo are templated by a random oligonucleotide during the ligation reaction.

Extending the AND logic to the splints further increases specificity when compared to approaches that use primer and padlock oligos, but where only the padlock carries barcode information. In CRISPRmap we distributed the barcode across the primer and padlock detection oligos such that we can identify possible self-ligations of the padlock (only positive for 2 readout probes) or unallowed combinations of padlock and primer oligos. Collectively the AND requirement for the primer, padlock and 2 splint oligos is likely to promote specificity. CRISPRmap lacks a reverse transcription or gap fill reaction typically required for in situ sequencing approaches; which likely contributes to increased efficiency of detection.

Of note, the degree of multiplexed phenotypic readout of our approach can be further expanded for both protein and transcriptomic detection. In this study, while we only profiled 12 transcripts, our RNAmap design is extendable to detect the expression of hundreds to thousands of genes, similar to recent hybridization-based optical transcriptomic approaches^{18-20,28}, which have profiled the expression of hundreds to thousands of genes, although such approaches can have practical limits associated with optical crowding and limited dynamic range. Multiplexed immunofluorescence assays, such as IBEX, have achieved concurrent profiling of ˜60-100 protein targets²². In expanding the antibody panel for large scale screens, careful consideration should be given to avoid tissue damage during cyclic staining, and steric hindrance between antibody panel members. This study enriches our understanding of gene functions by enabling systematic examination of spatial phenotypes within perturbed cells—attributes like morphology and subcellular localization of proteins that are lost in sequencing-based methodologies.

Our approach reduces the costs associated with barcode readout, and minimizes reliance on proprietary sequencing reagents. Moreover, CRISPRmap is flexible in the choice of readout dyes so they can be matched to existing microscopy setups available to researchers, encouraging broad adoption in the community. In addition to cancer cell lines profiling in vitro and in vivo, CRISPRmap is also applicable in primary cells, stem cells, induced pluripotent cells, and neurons derived from pluripotent cells, expanding the applicability of optical pooled screens to more challenging cell types in comparison to conventional OPS approaches.

We applied CRISPRmap to investigate the functional consequences of nucleotide variants in genes critical for the DNA damage response. Our multi-modal profiling of approximately 1 million cells enables a nuanced interrogation of how these variants influence cellular response to DNA damage by expanding the phenotypic profiling from fitness or a single fluorescent reporter to measuring dozens of DDR genes at the proteomic and transcriptomic level with subcellular spatial resolution. This enhanced view of the DDR response empowered us to identify missense variants of uncertain clinical significance whose DDR response resembles known pathogenic nonsense or splicing variants more closely than most VUS or unknown missense variants. Notably, our study was carried out in a breast cancer cell line, a disease for which further annotating VUS variants for their pathogenic potential is likely to help patients prioritize therapeutic strategies. As such, our approach can provide a framework for annotation of human variants in a treatment specific manner, and can help prioritize therapeutic strategies.

Recently, an elegant use of triplet combinations of linear epitopes enabled antibody-based identification of CRISPR guide expression of dozens of different guide expressing cancer cell populations within a tumor tissue at single-cell resolution and tissue scale¹⁷. RNA based barcoding offers an opportunity to increase library complexity to genome scale²⁹, but to date has not been established in a tissue context. We expect that CRISPRmap can be scaled up to accommodate larger screening library sizes. The set of 54 primer and 54 padlock oligos used in this study enables up to 2916 barcodes, but scaling up to 231 primer and padlock probes would encode for 53,361 barcodes, and thus enable genome-wide screening with 2 sgRNAs per protein coding gene, which could be read out using 44 20mer readout probes across 15 barcode readout cycles. While genome wide screens are feasible in in vitro setting, in vivo studies should carefully consider how many different perturbations can be profiled in a given mouse model, and how much tissue should be profiled to establish phenotypic significance. As reported in this study, CRISPRmap can detect ample barcode signal in a tissue context with subcellular resolution, which enables the interrogation of cells with their neighbors, and their surrounding extracellular milieu as a function of precise genome editing. Another opportunity for RNA based barcoding is to start exploring the effects of combinatorial gene perturbations, enabled by the spatial resolution of the barcode which offers the capability to detect more than one barcode expressed per cell. This capability could be helpful for screens to prioritize combinatorial targets, and inform therapy modalities that go beyond single-therapy strategies.

However, our study is not without limitations. RNA-based barcoding approaches are reliant on the stability of RNA molecules, which can be challenging in a tissue context due to the presence of RNAse enzymes. A possible resolution for this reliance is to transcribe barcode carrying RNA in vitro by T7 polymerases post hoc, as was recently reported for in vitro cells³⁰ This approach enables deep phenotypic profiling of tissue prior to in vitro generation of barcode carrying RNA molecules that can subsequently be detected by CRISPRmap. Moreover, although our RNAmap profiling approach has demonstrated good specificity, the detection efficiency is lower than traditional smFISH approaches. RNAmap allows for strong signal amplification, which enables high throughput imaging (20× objective and short exposure times) thus enabling visualizing millions of cells needed for large scale screening. For any study at hand, it would be of interest to evaluate the balance of deeper transcriptomic profiling enabled by FISH based approaches and throughput and scale of the screen. We expect CRISPRmap to be fully compatible with FISH based transcriptomic profiling. These constraints highlight the need for continual refinement of optical screening techniques and computational analysis methods.

The versatility of CRISPRmap may be expanded to include a broader range of CRISPR modalities, cell types and tissue environments. Studies can also delve deeper into the impact of genetic perturbations on tissue architecture and the interplay between cells in complex microenvironments. CRISPRmap paves the way for high-throughput investigations of gene function in diverse biological contexts, from developmental biology to the study of disease pathogenesis.

In conclusion, CRISPRmap offers a new lens through which we can examine the intricate tapestry of gene function within and across cells. Our findings herald a shift towards more spatially and temporally resolved studies of gene function, especially in tissue environments, potentially illuminating new paths in precision medicine and the quest to understand the underpinnings of complex biological systems.

### Materials and Methods

### Cell Lines and Cell Culture

HEK293FT cells (Thermo Fisher Scientific R70007) were cultured in DMEM (Gibco 11965092) supplemented with heat-inactivated 10% fetal bovine serum (FBS) (ATCC 30-2020) and 100 U/ml penicillin-streptomycin (Thermo Fisher Scientific 15140163). MCF7-BE3 cells were cultured in the same medium supplemented with 2 μg/ml Blasticidin (Thermo Fisher Scientific A1113903), HT1080/Cas9 AAVS1 (Genecopoeia SL512) cells were cultured in the same medium supplemented with 200 μg/ml HygromycinGold B (Invivogen ant-hg-2). OE19-BFP cells were cultured in RPMI-1640 Medium (ATCC 30-2001) supplemented with 10% heat-inactivated FBS, 100 U/ml penicillin-streptomycin, lx Glutamax supplement (Thermo Fisher Scientific 35050079) and 2 μg/ml Blasticidin. IMR-90 (ATCC CCL-186) cells were cultured in EMEM (ATCC 30-2003) supplemented with heat-inactivated 10% FBS and 100 U/ml penicillin-streptomycin. Rockefeller University Embryonic Stem Cell Line 2 (RUES2, passage 24-32) were maintained on mouse embryonic fibroblasts (MEFs) (Thermo Fisher Scientific A34180) and plated at 22,500 cells/cm². Cells were cultured in hESC maintenance media (DMEM/F12 (Thermo Fisher Scientific 11320033), 20% Knock-out serum (Stem Cell Technologies, Vancouver, BC), 0.2% Primocin (InvivoGen ant-pm-05), 0.1 mM P-mercaptoethanol (Sigma-Aldrich M6250), 20 ng/ml FGF2 (R&D Systems 233-FB), and 1% Glutamax). The medium was changed daily. hESCs were passaged every 3-4 days with Accutase (Innovative Cell Technologies AT-104), washed and replated at a dilution of 1:24. Cultures were maintained in a humidified 5% CO₂ atmosphere at 37° C. Lines are karyotyped and verified for Mycoplasma contamination using PCR every 6 months. hESC were infected with the virus for 24h in hESC medium supplemented with polybrene and puromycin selected. Prior to analysis, MEFs were depleted by passaging 5-7×10e5 hESCs onto Matrigel (Corning 354277, dilution 1:15) coated flat glass bottom 96 well plates. Cells were maintained in hESC medium in a humidified 5% CO2 atmosphere at 37° C. For human iPSC and derived-motor neuron (iMN) experiments, we used reference Wt line KOLF2.1J³¹ (a gift from Dr. Christopher Ricupero). iPCSs were maintained on Matrigel (1:100, Corning 354277) coated plates in mTeSR Plus media (STEMCELL Technologies 100-0276); supplemented with Y-27632 (ROCKi, 10 μM, Selleckchem S1049) during thawing, passaging, and viral transduction. Passaging was performed using Accutase (Thermo Fisher A1110501). iPSCs were transduced with the CRSPRMap library during passaging by adding viral supernatant to polybrene (8 μg/ml Sigma-Aldrich TR-1003-G) supplemented media at various dilutions. 48 hours later, transduced cells were selected with puromycin (1 μg/ml, Thermo Fisher A113802) for 3 days. For optical barcode detection in iPSCs, iPSCs were dissociated into single-cells and plated on polyethylenimine (PEI, Sigma 408719) coated 96-well plates for imaging in mTeSR Plus with ROCKi at 10,000 cells per well. ROCKi was maintained prior to fixing, preventing tight colony formation to simplify cell segmentation during image analysis. For coating, 96-well plates were incubated at 37° C. overnight with PEI (250 μg/mL, 50 μL/well), then washed at least 3 times with 200 μL/well PBS.

iPSC to iPSC-derived motor neuron (iMotorNeuron) differentiation was carried out as previously described^{32,33}. Briefly, on day 0, iPSCs were dissociated to single cells with Accutase and resuspended in N2B27 differentiation media (1:1 Advanced DMEM/F12 and Neurobasal media (Life Technologies 12634010, 21103049), Glutamax (1%, 35050061), beta-mercaptoethanol (0.1%, Sigma), N-2 (1%, Thermo Fisher 17502048), B-27 (2%, Thermo Fisher 17504044), and ascorbic acid (10 μM, Sigma A4403) supplemented with ROCKi (10 μM), FGF2 (10 ng/mL, Peprotech PHG0263), CHIR99021 (CHIR, 3 μM, Tocris 4423), SB 431542 hydrate (SB, 20 μM, Sigma S4317), LDN193189 (LDN, 100 nM, Stemgent 04-0074) at a density of 50,000 cells/mL on ultra-low adhesion dishes to promote embryoid body (EB) formation. On day 2, media was replaced, supplemented with CHIR (3 μM), SB (20 μM), LDN (100 nm), all-trans retinoic acid (RA, 100 nM, Sigma R2625), smoothened agonist (SAG, 500 nM, Millipore 566660). On day 4, media was replaced, supplemented as on day 2. On day 7, media was replaced, supplemented with RA (100 nM), SAG (500 nM), and BDNF (10 ng/mL Peprotech 450-02). On day 9, media was replaced, supplemented with RA (100 nM), SAG (500 nM), BDNF (10 ng/mL), and DAPT (10 μM, Selleckchem S2215). On day 11, media was replaced, supplemented as on day 9. On day 14, media was replaced, supplemented with RA (100 nM), SAG (500 nM), BDNF (10 ng/mL), DAPT (10 μM), and GDNF (10 ng/mL, R&D Systems 212-GD-050). On day 16, EBs were dissociated to single cells by trituration with 0.05% Trypsin (Life Technologies 25300054). Dissociated MNs were resuspended in hMN maintenance media (Neurobasal media, Glutamax (1%), NEAA (1%, Life Technologies 11140050), beta-mercaptoethanol (0.1%), N-2 (1%), B-27 (2%), ascorbic acid (10 μM), BDNF (10 ng/mL), GDNF (10 ng/mL), CNTF (10 ng/mL, Peprotech 257-NT-050), IGF-1 (10 ng/mL, Peprotech 291-G1), RA (1 μM) and Adarotene (1 μM, MedChem Express HY-14808)) and plated on PEI coated 96-well plates for imaging at 10,000 cells per well.

After CRISPRmap amplicon generation, cell type validation was performed on iPSCs and iMotorNeurons (iMNs) by immunostaining using anti-SOX2 (iPSC marker, 1:200, Thermo Fisher Scientific 14-9811-82), anti-OCT4 (iPSC marker, 1:200, Cell Signaling Technology 2840) and anti-NeuN (iMN marker, 1:200, Millipore Sigma MAB377) antibodies. Likewise, cell type validation was performed on hESCs using anti-SOX2 (hESC marker, 1:200), anti-OCT4 (hESC marker, 1:200) and anti-Nanog (hESC marker, 1:200, Cell Signaling Technology 4893) antibodies.

### Library Design and Cloning

GFP targeting guides in the GFP-targeting CRISPRmap knockout screen library (referred as GFP-pilot) were designed with CRISPick³⁴ by selecting the top 5 recommended candidates in the CRISPRko mode that target the copGFP sequence. The library also contains five non-targeting control (NTC) guides that lack targets in the human genome. Each guide was combined with a universal scaffold sequence and a pair of guide-specific CRISPRmap barcode sequences. Universal 5′ and 3′ homology sequences were then added to facilitate NEB HIFI assembly into the expression vector. Full-length GFP-pilot library sequences are shown in Table 5. Base-editing guides in the DNA damage response screen library (referred as DDR364) were selected from the base-editing screens as previously described⁷. The library contains 162 missense guides and 50 nonsense guides with a single C base in the editing window (4^{th} to 8^{th} base in the guide targeting sequence), 80 splice-donor or splice-acceptor (referred as splice) guides, 35 guides targeting the AAVS1 safe-harbor site, and 37 non-targeting control guides that have minimal targets in the human genome. All the selected missense, nonsense and splice guides have FDR<0.05 in at least one treatment in the previous screen⁷. Similarly, each guide was combined with the scaffold, CRISPRmap barcode, and homology sequences. Sequences are shown in Table 6. Both libraries were ordered as synthesized oligo pools (Integrated DNA Technologies) and PCR-amplified with Q5 DNA polymerase (New England Biolabs M0492) using an optimized two-round amplification strategy to minimize barcode-sgRNA recombination³⁵. Briefly, oligo pools were diluted in ultrapure water (Thermo Fisher Scientific 10-977-023) and 1 μg of total DNA was added to each 50 μl Q5 reaction mix to perform the first-round amplification of 15 PCR cycles, 0.5 μl PCR product from each 50 μl first-round reaction was then added to each 50 μl Q5 reaction mix for the second-round amplification of 10 cycles. Final PCR product was purified with DNA clean & concentrator (Zymo Research D4013). The primer pairs CRISPRmap-F and CRISPRmap-R in Table 7 were used in both rounds. Amplified oligo pools were cloned into a modified CROPseq-puro-v2 (Addgene #127458) vector that removed the original scaffold sequence (referred as CRISPRmap-CROPseq) using NEBuilder HiFi DNA Assembly (New England Biolabs E2621). Next, we electroporated into MegaX DH10B electrocompetent Cells (Thermo Fisher Scientific C640003). An average number of 300 colonies per guide was maintained to preserve the relative abundance of guides in the library. Bacterial colonies were scraped and pooled for plasmid extraction (Zymo Research D4212).

### Lentivirus Production & Titer Determination

293FT cells were seeded into 6-well tissue culture-treated plates at a density of 100,000 cells/cm². After 24 hours, cells were transfected with pMD2.G (Addgene #12259), psPAX2 (Addgene #12260), and CRISPRmap library plasmid (2:3:4 ratio by mass) using Lipofectamine 3000 (Thermo Fisher Scientific L3000001) in Opti-MEM (Thermo Fisher Scientific 31-985-070) supplemented with 5% FBS. Media was exchanged after 6 hours and supplemented with 1.5 mM caffeine (Sigma-Aldrich C0750) to increase viral titer. Viral supernatant was harvested at 24 hours and 48 hours after transfection, filtered through 0.45 μm cellulose acetate filters (Corning 431220) and stored in −80° C. freezer in aliquots.

Lentiviral titer was determined by the colony formation assay to control the multiplicity of infection (MOI) in downstream studies. Briefly, 10-fold serial dilutions of the lentivirus stock were prepared in complete DMEM medium containing 8 μg/mL polybrene. 10,000 cells were seeded into each well of a six-well plate. A total volume of 1 mL diluted lentivirus was added to each well for 48 hours. Cells were then cultured in complete DMEM media supplemented with appropriate antibiotics for 14 days and media were changed every 3 days. Cells were fixed and stained with 0.1% crystal violet (Sigma-Aldrich V5265) for 10 minutes at room temperature and washed three times with PBS. Colonies on each well were counted and the transduction units per mL (TU/mL) was calculated as follows: TU/mL=number of colonies/total volume in the well (mL)×dilution factor.

### Fluorescence Microscopy

All imaging datasets were acquired using a confocal spinning disk microscope (Andor Dragonfly) coupled to a Nikon Ti-2 inverted epifluorescence microscope with automated stage control, Nikon Perfect Focus System, and a Zyla PLUS 4.2 Megapixel USB3 camera. Illumination was done with 100 mW 405 nm, 50 mW 488 nm, 50 mW 561 nm, 140 mW 640 nm and 100 mW 785 nm solid state lasers. All hardware was controlled using Andor Fusion software. Lasers, laser powers, exposure times, objectives and experiment-specific acquisition parameters are summarized in Table 5 & Table 6. Images were acquired with 4 z-slices at 1.5 μm intervals for the cultured cells and with 6 z-slices at 1.5 μm intervals for the tissue sections unless otherwise specified.

### Oligonucleotide Fluorophore Conjugation

In each 10 μl reaction, 2 μl of 0.5 mM 5′ amine-modified DNA probes (Integrated DNA technologies) is mixed with 1 μl of 10 mM ATTO488-NHS ester (ATTO-TEC AD 488-31), ATTO 643-NHS ester (ATTO-TEC AD 643-31) or CF568 succinimidyl ester (Sigma-Aldrich SCJ4600027) in 1×BBS (Thermo Fisher Scientific 28384) pH 8.5, and incubate at room temperature for 4 hours. Fluorophore-conjugated DNA probes were purified with Oligo Clean & Concentrator (Zymo Research D4060), and diluted to 1 μM in ultrapure water, aliquoted and stored at −20 C. Oligonucleotide sequences and fluorophores used in the GFP-targeting screen were listed in Table 5, the base-editing screens and in vivo CRISPRmap barcode readout were listed in Table 6.

### Antibody Fluorophore Conjugation

In each conjugation reaction, 5 μg of antibody in PBS (BSA-free) is mixed with 1 μl 0.33 mM CF750 Dye SE/TFP esters (Biotium 92142), Alexa Fluor 647 NHS Ester (Thermo Fisher Scientific A20006), Alexa Fluor 555 NHS Ester (Thermo Fisher Scientific A20009), or Alexa Fluor 488 NHS Ester (Thermo Fisher Scientific A20000) in DMSO, and incubated at room temperature for 16 hours. Fluorophore-conjugated antibodies were then purified with 30 kDa Amicon Ultra-0.5 Centrifugal Filter Unit (Millipore Sigma UFC5030BK) Antibodies used in the base-editing screen and in vivo barcode readout are listed in Table 6.

### CRISPRmap Optical Pooled CRISPR Knockout Screen

HT1080/Cas9 AAVS1 cells were seeded into 6-well tissue culture-treated plates at a density of 50,000 cells/cm². After 24 hours, cells were transduced with the GFP-pilot lentiviral supernatant supplemented with 8 μg/ml polybrene at MOI ˜0.1. At 48 hours post-infection, viral supernatant was removed and cells were treated with media containing 2 μg/ml puromycin for 48 hours and seeded onto 96-well glass bottom plates (Cellvis P96-1.5H-N) at 10,000 cells per well as the original seeding density. Cells were seeded at 4,000 cells per well as the sparse density to avoid extensive overlapping among cells. A total reaction volume of 50 μl was used in the following steps unless otherwise specified. After 24 hours, cells were fixed in 4% paraformaldehyde (Electron Microscopy Sciences 15710-S) in PBS (Gibco 10010049) for 10 minutes at room temperature, followed by two rinses in PBS. Cells were then incubated in 0.1 mg/ml Wheat Germ Agglutinin (WGA) CF770 conjugate (Biotium 29059) and 0.5 μg/ml DAPI (Abcam ab285390) in PBS for 30 minutes at room temperature and imaged in PBS for membrane, GFP, and nuclei signal using the microscope configuration described above. After phenotype imaging, cells were permeabilized with 0.2% Triton X-100 (Sigma-Aldrich T8787) in PBS for 10 minutes at room temperature, followed by two rinses in PBS. The permeabilization conditions are to be determined for each new cell type, as it is one of the parameters that determines barcode detection efficiency. For primer and padlock oligo hybridization, cells in each well were incubated in the Hybridization mix (GFP-pilot CRISPRmap Padlock and Primer mix (see Table 5 for sequences, each oligo in the mix has a final concentration of 10 nM), 1 mg/ml yeast tRNA (Invitrogen 15401011), 2×SSC, 20% formamide (v/v) in ultrapure water) for 16 hours at 40° C. in a HybEZ oven (ACD PN 321720). After hybridization, cells were first rinsed three times with the hybridization wash buffer (2×SSC, 20% formamide (v/v) in ultrapure water), then washed three times for 5 minutes at 40° C. Cells were then incubated in splint mix (10 nM CRISPRmap GFP-pilot splint mix (see Table 5 for sequences, each splint oligo in the mix has a final concentration of 10 nM), 0.1% yeast tRNA, 2×SSC and 15% formamide in ultrapure water) for 30 minutes at 37° C. in a HybEZ oven, rinsed twice with the formamide wash buffer (2×SSC, 15% (v/v) formamide in ultrapure water), and incubated in 2×SSC in ultrapure water for 15 minutes at room temperature. For T4 DNA ligation, cells were incubated in ligation mix (lx T4 ligase buffer, 1% (v/v) T4 DNA ligase (Enzymatics L6030-HC-L) in ultrapure water) for 2 hours at 16° C. then 1 hour at 25° C. in a HybEZ oven, followed by two rinses in PBS. For rolling circle amplification (RCA), cells were incubated in RCA mix (lx QualiPhi buffer, 2% (v/v) QualiPhi DNA Polymerase (4basebio 510100), dNTP mix, 0.25 mM each (Thermo R1122), 0.02 mM 5-(3-Aminoallyl)-dUTP (Thermo AM8439) in ultrapure water) for 6 hours at 30° C. then remove the RCA mix and immediately fix with 4% PFA in PBS for 10 minutes at room temperature, followed by three PBS washes. For readout probe hybridization, cells in each well were incubated in readout probe mix (10 nM of each readout probe (see readout probe sequences for each hybridization rounds in Table 5), 2×SSC, 15% formamide in ultrapure water) for 30 minutes at 37° C. in HybEZ oven. Cells were then imaged in the imaging buffer (0.5 μg/ml DAPI, 10 μg/ml Fungin (InvivoGen ant-fn-1) in PBS) using the microscope configuration described above. After imaging, the cells were incubated in the stripping buffer (2×SSC, 50% formamide (v/v) in ultrapure water) for 20 minutes at 40° C. in the HybEZ oven and then rinsed once with formamide wash buffer. A total of 4 readout hybridization rounds were performed to decode all the CRISPRmap barcodes in the GFP-pilot library. The same CRISPRmap assay and barcode detection protocol was applied to IMR-90, iPSCs, iMotorNeurons, and hESCs.

To quantify the sensitivity, specificity and precision of the assay, average fluorescence intensity of GFP under nuclei masks were quantified and a threshold was determined based on the GFP intensity distribution of the cell population to classify cells into GFP+ and GFP− categories. Standard CRISPRmap quality check (QC) was performed for each cell to determine the guide identity. Specifically, we quantified the spot count for the most representing guide-reporting barcode (max_spot) and the second most representing guide-reporting barcode (second_max_spot) in each cell, and a purity score is calculated by: Purity=max_spot/(max_spot+second_max_spot). Guide identity is only assigned to a cell (i.e. a cell passed QC) when the max_spot >=3 and Purity >=0.66. A relaxed QC metrics (max_spot>=2 and Purity>=0) was applied to enable a side-by-side comparison between CRISPRmap and Conventional OPS. The guide identity reported by the most representing barcode will be assigned to a cell passed QC. Ratio of cells passed QC is calculated as the ratio between the number of cells passed QC and the total number of cells profiled. To calculate sensitivity, specificity and precision, we define a True Positive (TP) as a GFP− cell is assigned with one of the GFP-targeting guides in the GFP-pilot library; a True Negative (TN) is defined as a GFP+ cell is assigned with one of the Non-targeting guides; a False Positive (FP) is defined as a GFP+ cell is assigned with one of the GFP-targeting guides; a False Negative (FN) is defined as a GFP− cell is assigned with one of the Non-targeting guides. Specificity=TN/(TN+FP); Sensitivity=TP/(TP+FN); Precision=TP/(TP+FP). QC metrics from loose to tight were applied in FIGS. 23C-E.

### CRISPRmap Barcode Readout Optimization and Quantification

To optimize and quantify the CRISPRmap barcode readout, we created two HT1080 cell lines with fluorescent protein (FP) expression tethered to the CRISPRmap Barcode reporting on the FP identity: one cell line expresses GFP, a non-targeting control guide (NT_GFP) and the CRISPRmap Padlock and Primer hybridization sequence for Padlock003 and Primer003 (GFP_Barcode), the other cell line expresses mTurquoise2, a non-targeting control guide (NT_mTurquoise2) and the CRISPRmap Padlock and Primer hybridization sequence for Padlock004 and Primer004 (mTurquoise2_Barcode). The sequences are listed in Table 5. FPs were introduced to the CRISPRmap-CROPseq vector by replacing the Puromycin resistance gene. The sgRNA-Barcode (NT_GFP+GFP_Barcode, NT_mTurquiose2+mTurquoise2_Barcode) sequences were ordered as synthesized double-strand DNA fragments (integrated DNA Technologies) and cloned onto CRISPRmap-CROPseq vector replaced with GFP and mTurquoise2, respectively. Plasmid-EZ sequencing (Azenta Life Sciences) was performed to confirm the sgRNA-Barcode combination matches with the FP expressed, prior to lentiviral packaging and infection on the HT1080 cells. The two cell lines were sorted by flow cytometry, mixed at 1:1 ratio and seeded onto 96-well glass bottom plates for genotype-phenotype mapping. Cells were fixed in 4% PFA in PBS for 10 minutes at room temperature, then incubated with DRAQ5 fluorescent probes (0.05 mM, Thermo Fisher Scientific 62251) and Wheat Germ Agglutinin (WGA)-CF770 (10 ug/mL) for 20 minutes at room temperature for nucleus and membrane segmentation, respectively. WGA-CF770, DRAQ5, GFP and mTurqoise2 fluorescence signals were imaged with 730 nm, 640 nm, 488 nm and 405 nm laser, respectively. Cell permeabilization, amplicon generation, barcode readout and image analysis were performed, as described in the CRISPRmap pooled CRISPR knockout screen for the GFP-pilot library. Average fluorescence of GFP and mTurquiose2 signals under nuclei masks were quantified to classify cells into GFP+ and mTurquoise2+ categories, and the FP identity in each cell was matched to the detected barcodes to evaluate the sensitivity, specificity and precision of the assay (FIGS. 23C-E, Table 2).

To quantify the CRSPRmap barcode readout for potential double-transduced cells, we performed lentiviral infection of the GFP-pilot library at different multiplicity of infection (MOI) of 0.9, 0.3, 0.1, and 0.03 on HT1080 cells. Cells were puromycin selected, seeded and profiled for barcode expression, as described above. To identify potential double transduced cells, we first performed standard QC to identify cells with unique barcodes, then classified the remaining cells to be “double” if the spot count for the most representing guide (max_spot) >=3 and the second most representing spot (second_max_spot) >=2. The expected ratio of double-transduced cells after antibiotic selection at a given MOI was calculated by Poisson distribution after removing the proportion of cells with zero infection event. The ratio of double-transduced cells detected optically and the expected ratio at each MOI were shown in FIG. 23L, Table 2.

### CRISPRmap Multimodal Optical Pooled Base-Editing Screen

MCF7-BE3 cells were transduced with the DDR364 library in the same manner as in the GFP-targeting knockout screen with several modifications to accommodate the multiplexed immunofluorescence and RNAmap. Specifically, after puromycin selection, cells were seeded onto 6-well glass bottom plates (Cellvis P6-1.5H-N) at a density of 50,000 cells/cm2. For the DDR364-irradiation screening, after 48 hours, cells were exposed to 10 Gy ionizing radiation using the Gammacell 40 cesium source irradiator and fixed at 6 hours after irradiation. For the DNA damaging agents (DDR364-chemo) screening, cells were treated with 100 nM Camptothecin (Sigma-Aldrich C9911), 1 μM Olaparib (Selleck Chemicals S1060), 1 μM Cisplatin (Sigma-Aldrich P4394), 1 μM Etoposide (Sigma Aldrich E1383), or untreated, and fixed at 24 hours post-treatment. After fixation, cells were permeabilized with 0.1% Triton-X100 in PBS for 10 minutes on ice. Cells in each well were incubated in 1 ml reaction mix or buffers in all steps unless otherwise specified. After permeabilization, cells were incubated in the antibody mix (2 μg/ml rat anti-CD326 (BioLegend 312502), 1 μg/ml rabbit anti-RAD51-AF647 (Bioacademia 70-012), 2 g/ml mouse anti-BRCA1-AF555 (Santa Cruz Biotechnology sc-6954), 0.5 μg/ml rabbit anti-RPA2-AF488 (Bethyl Laboratories A300-244A) in PBS) for 1 hour at room temperature, then rinsed twice with PBS. Cells were incubated in 10 μg/ml goat anti-Rat-IgG secondary antibody (Thermo Fisher Scientific SA5-10023) for 30 minutes at room temperature, and rinsed twice with PBS. Cells were fixed in 4% PFA in PBS for 10 minutes at room temperature to cross-link the antibodies to the cells, followed by two PBS rinses. Cells were then processed with padlock and primer probes hybridization, splint hybridization, ligation and RCA as described above, with the minor difference that 3 nM of each CRISPRmap Padlock and Primer probes and 3 nM of each RNAmap Padlock and Primer probes were used in the hybridization mix. Probe sequences are listed in Table 6 After RCA and fixation, cells were first imaged in the imaging buffer for membrane, nuclei, and nuclear foci signal using the microscope configuration described in Table 6. After imaging, the antibody signal was bleached with 1 mg/ml lithium borohydride (Sigma-Aldrich 222356) and rinsed twice with PBS, prior to the incubation of the next round of antibodies. For both the DDR364-irradiation and the DDR364-chemo screening, a total of 4 antibody incubation-bleaching rounds were performed. After the last round of bleaching, eight rounds of RNAmap readout probe hybridization-stripping rounds were performed, followed by eight rounds of CRISPRmap readout probe hybridization-stripping rounds. Each round was imaged using the microscope configuration described above. Readout probe sequences and conjugated fluorophores are listed in Table 6. For the DDR364-irradiation screening, cells were incubated in Vector TrueVIEW Autofluorescence Quenching reagent (Vector Laboratories SP-8400-15) for 5 minutes at room temperature to reduce autofluorescence, followed by three rinses in PBS before imaging each CRISPRmap readout round in high DAPI imaging buffer (2.5 μg/ml DAPI, 10 μg/ml Fungin in PBS).

### In Vivo CRISPRmap Barcode Readout and Multi-Modal Phenotyping

Following lentiviral transduction with pLV-EF1a-TagBFP2 on OE19 cells, fluorescence-activated cell sorting (Sony MA900) was performed to obtain a BFP-expressing OE19 population. BFP-expressing OE19 (referred as OE19-BFP) cells were lentiviral transduced with the DDR364 library and puromycin selected as described above, then expanded for 4 days in puromycin-free media. We suspended 5×10⁶ cells in a 1:1 mixture of Matrigel and PBS and inoculated the mixture into the flanks of nude mice (JAX, strain no. 002019). Mice were housed with a constant temperature of 21-24° C., 45-65% humidity and a 12-h light-dark cycle. After 17 days, tumors were harvested and fresh frozen in OCT on dry ice and stored at −80° C. Frozen tumor samples were sectioned in Cryostat Microtome (Leica CM1510S) at −20° C. into 10 μm thick sections, and deposited onto 12-well glass-bottom plates (Cellvis P12-1.5H-N) coated with 0.1 mg/ml poly-D-lysine (Sigma-Aldrich A-003-E). CRISPRmap barcode readout and antibody staining were performed as described above with minor modifications. Specifically, 400 μl of reaction mix and buffers were added to each well to fully cover the tissue section. Tissue sections were fixed with 4% PFA in PBS for 15 minutes at room temperature and permeabilized with 0.5% Triton X-100 in PBS for 15 minutes at room temperature. 30 nM of each CRISPRmap padlock and primer oligos were used in the hybridization mix. The same set of CRISPRmap padlock and primer probes, splints, and readout probes was used as in the DDR364-irradiation screening. Eight CRISPRmap readout cycles were performed prior to antibody staining and bleaching cycles. The same readout probes were used as in the base-editing screens.

### Conventional Optical Pooled Screens (OPS)

The conventional OPS on cultured cells (HT1080, IMR-90, iPSCs, hESCs, and motor neurons) were performed in accordance with the published protocol^{3,4}. Briefly, cells were fixed and permeabilized in the same conditions with cells undergoing the CRISPRmap protocol for side-by-side comparisons. Specifically, cells were fixed in 4% PFA in PBS for 10 minutes at room temperature and permeabilized with 0.2% Triton X-100 in PBS for 10 minutes at room temperature. Reverse transcription mix (lx RevertAid RT buffer, 250 μM dNTPs, 0.2 mg/mL BSA (New England Biolabs B9000S), 1 μM RT primer (/5AmMC12/A+CT+CG+GT+GC+CA+CT+TTTTCAA (SEQ ID NO: 1), Integrated DNA Technologies), 0.8 U/μL Ribolock RNase inhibitor (Thermo Fisher Scientific EO0384), and 4.8 U/μL RevertAid H minus reverse transcriptase (Thermo Fisher Scientific EP0452)) was added to the cells and incubated for 16 hours at 37° C. Cells were washed 5 times with PBS-T and fixed with 3% paraformaldehyde and 0.1% glutaraldehyde in PBS for 30 minutes at room temperature, then washed with PBS-T 5 times. Cells were incubated with the gap-fill reaction mix (lx Ampligase buffer, 0.4 U/μL RNase H (Enzymatics Y9220L), 0.2 mg/mL BSA, 100 nM padlock probe (/5Phos/GTTTCAGAGCTATGCTCTCCTGTTCGCCAAATTCTACCCACCACCCACTCTCC AaaggacgaaaCACC (SEQ ID NO: 2), Integrated DNA Technologies), 0.02 U/μL TaqIT polymerase (Enzymatics P7620L), 0.5 U/μL Ampligase (Lucigen A3210K) and 50 nM dNTPs) for 5 minutes at 37° C. and 90 minutes at 45° C., washed twice with PBS-T, then incubated with the rolling circle amplification mix (1×Phi29 buffer, 250 μM dNTPs, 0.2 mg/mL BSA, 5% glycerol, and 1 U/μL Phi29 DNA polymerase (Thermo Fisher Scientific EP0091)) at 30° C. for 16 hours. For in situ sequencing, 1 μM sequencing by synthesis primer (GCCAAATTCTACCCACCACCCACTCTCCAaaggacgaaaCACC (SEQ ID NO: 3), Integrated DNA Technologies) in 2×SSC were added to the cells for 30 minutes at room temperature. Incorporation mix (Illumina MS-103-1003, MiSeq reagent #1) were added to the cells for 5 minutes at 60° C., and the cells were rinsed 5 times in PR2 and washed by 5 cycles of 5 min 60° C. washes. Cells were imaged using illumination of 100 mW 405 nm (DAPI), 50 mW 488 nm (G base), 50 mW 561 nm (C base) and 140 mW 640 nm (A base) lasers. Cells were then incubated in the cleavage mix (Illumina MS-103-1003, MiSeq reagent #4) at 60° C. for 6 minutes, followed by three rinses with PR2, one wash with PR2 at 60° C. for 1 minute, and three rinses with PR2 again, before entering the next incorporation step. Four bases were sequenced in order to distinguish the guide sequences in the GFPpilot library. Sensitivity, specificity, and precision were calculated based on the barcode identity and GFP expression level in each cell, as described in the CRISPRmap pooled CRISPR knockout screen for the GFP-pilot library.

### Variant Annotation

The sgRNA category of each guide was annotated as previously described⁷. We grouped the splice-donor and splice-acceptor categories into a ‘splice’ category. All AAVS1-targeting and non-targeting guides are annotated as a ‘control’ category. The ClinVar category was determined by querying each guide in the ClinVar database (v. 2023-12-15; https://www.ncbi.nlm.nih.gov/clinvar/; RRID:SCR_006169). Nonsense and missense variants were queried based on the specific amino acid change, whereas splice variants were queried based on the nucleotide change outcomes in the editing window (base C in the 4th to 8th bases in the sgRNA targeting sequences). Note that if multiple C bases exist in the editing window, a splice guide can render other mutational outcomes, such as missense or intron variants. These mutational outcomes were not counted in the annotation of splice variants but listed as “Less deleterious variants” in Table 6. The determining criteria of the Clinvar category were established as previously described⁷. Briefly, three categories were assigned to non-control guides: i) benign/likely-benign (B/LB); ii) variants of uncertain significance (VUS); iii) pathogenic/likely-pathogenic (P/LP). The VUS category also includes variants with conflicting interpretations. If a variant was not documented in the ClinVar database, it was listed as “unknown”.

### Library Quality-Check and Quantification by Next Generation Sequencing

For oligo pool quantification, the first-round amplification product in the library cloning step was collected and 0.5 μl of 50 μl PCR product was added to each 50 μl Q5 reaction mix for the second-round amplification of 10 cycles using the primer pairs CRISPRmap-F-ad and CRISPRmap-R-ad in Table 7. We amplified 10 pg plasmid extraction product from the library cloning step with the same two-round strategy as the oligo pool quantification for plasmid-level quantification. Genomic DNA of the cells transduced with the sgRNA library were extracted with Genomic DNA Clean & Concentrator (Zymo Research D4010). We amplified 100 ng genomic DNA with the same two-round strategy. We had 5 ng of the final PCR product sequenced with next generation sequencing (Azenta Life Sciences Amplicon-EZ). sgRNA sequences in the library were aligned to the NGS reads to quantify the relative abundance of each guide in the library and the padlock and primer hybridization sequences (Barcodes) were aligned to each NGS read containing a valid sgRNA sequence to evaluate the barcode-sgRNA recombination rate. Each read with a valid sgRNA sequence were classified into “matched” (sgRNA-Barcode combination matched the codebook), “switched” (sgRNA-Barcode combination does not match the codebook), “loss of BC” (no valid padlock or primer sequences detected), or “unallowed BC” (unallowed padlock and primer combination detected) category. The results are shown in FIG. 23J, Table 2.

### Base-Editing Screen Hits Validation

Individual sgRNAs with the same guide and scaffold sequences as used in the base-editing screen were ordered as synthesized double-strand DNA fragments (integrated DNA Technologies) and cloned onto the CRISPRmap-CROPseq vector. As described in the base-editing screening, cells transduced with individual sgRNAs were selected for 2 days in puromycin and cultured for 2 days before ionizing radiation. Six hours after irradiation, cells on the glass-bottom plates were fixed for immunostaining of the same panel of nuclear foci imaged in the screen. Cells on tissue culture plates were harvested for immunoblotting. Genomic DNA was extracted from the untreated cells with QuickExtract DNA Extraction Solution (Lucigen QE09050) at the same time point for evaluating base-editing efficiency of the individually transduced guides. PCR amplification was performed on the genomic locus of the intended base edit (Primer sequences listed in Table 7) using Q5 DNA polymerase (New England Biolabs M0492), followed by sanger sequencing (Azenta Life Sciences). ICE analysis (Synthego Performance Analysis, ICE Analysis. 2019) was performed on the sanger sequencing results to quantify the in-window and out-of-window editing efficiency (FIG. 34A).

### Immunoblotting

Cells transduced with individual sgRNAs were selected for 2 days in puromycin and cultured for 2 days before collection as described in the base-editing screening. Cells treated with siRNAs were subjected to reverse siRNA transfection utilizing of firefly (FF) siRNA, BRCA1 siRNA, or BRCA2 siRNA at 20 nM and lipofectamine RNAiMAX (Thermo Fisher Scientific 13778075) as per manufacturer's indications. Cells were trypsinized, washed and resuspended in sample buffer (0.1M Tris pH 6.8, 4% SDS, 12% P-mercaptoethanol) at a density of 20,000 cells/μl. Subsequently, samples were sonicated for 10 seconds twice and boiled at 95° C. for 5 min prior to gel electrophoresis. After gel electrophoresis, proteins were transferred onto nitrocellulose membranes. Proteins were detected using the appropriate primary and HRP-conjugated secondary antibodies at a 1:10,000 dilution. Primary antibodies used in this study include mouse-anti-BRCA1 (Santa Cruz Biotechnology sc-6954, 1:100), rabbit anti-phospho-KAP1 (Bethyl Laboratories A700-013, 1:1,000), rat anti-Tubulin (Novus Biologicals NB 600-506, 1:50,000), and mouse anti-BRCA2 (Millipore OP95, 1:1,000).

### RNA Sequencing

Gamma-irradiated and untreated MCF7-BE3 cells were prepared in parallel to the cells profiled in the DDR364-irradiation screen. Six hours after irradiation, total RNA was extracted with Quick-RNA Microprep Kit (Zymo Research R1051), and mRNA was isolated with NEBNext Poly(A) mRNA Magnetic Isolation Module (New England BioLabs E7490L). RNA integrity number (RIN) was quantified with RNA Pico 6000 assay (Aligent 5067-1513) on BioAnalyzer (Aligent 2100 G2939BA). DNA libraries for Next Generation Sequencing were prepared with NEBNext Ultra II RNA Library Prep Kit for Illumina (New England BioLabs E7775) and NEBNext Multiplex Oligos for Illumina (Unique Dual Index UMI Adaptors RNA Set 1) (New England BioLabs E7416). DNA libraries were quality checked with DNA 1000 assay (Aligent 5067-1504) on Bioanalyzer, then sequenced on a Miseq platform (Illumina) with a 5% spike-in of PhiX (Azenta Life Sciences, Sequencing-Only). Four replicates were sequenced and the average Transcript per Million reads (TPM) was calculated for the transcripts we profiled optically with RNAmap.

### CRISPRmap and RNAmap Primer and Padlock Probe Design

The gene-specific target probes for RNAmap are designed for specificity, and minimized off-target binding, conforming to SeqFISH methodologies^{28,36}, utilizing the FISHprobe R package (v0.4.1; https://github.com/stevexniu/fishprobe). For Gene Selection and Probe Extraction, we selected highly expressed gene isoforms from the Human GTEX V8³⁷ and Mouse ENCODE³⁸ tissue expression datasets for probe design. Probe sequences, 20-30 nucleotides in length, were derived from the coding sequence (CDS) and, where necessary, from the untranslated regions (UTRs). We targeted a GC content range of 45%-65% or 30%-70% for the targeting probes, excluding those with unsuitable GC content or sequences prone to forming homopolymeric runs (such as G-quadruplexes) to maintain optimal hybridization characteristics.

Specificity and Off-Target Minimization: Local BLAST³⁹ searches against human and mouse mRNA sequence databases identified probes with off-targets, particularly those with alignments exceeding 10-15 nucleotides with unrelated genes in the transcriptomes and the repetitive DNA using repetitive masks. Tissue-specific expression data from human³⁷ or mouse³⁸ were pivotal in developing a gene copy-number table for each tissue type, which informed the exclusion of probes with off-target copy numbers exceeding 15-20 log TPM. For thermal stability and structural integrity, to refine the probe pool by optimizing GC content for enhanced binding affinity, an iterative selection process was employed. Probes were initially ranked in ascending order of their deviation from the target GC content of 55%, starting with the probe exhibiting the greatest deviation. This arrangement continued until no overlapping probes remained. Subsequently, the selection process took into account the calculated melting temperatures (Tm)⁴⁰. For secondary structure predictions, including pseudoknots, the analysis was conducted under specific conditions: a sodium ion concentration of 0.33 M (equivalent to 2×SSC), and 50% formamide at 37° C.⁴⁰. Probes with an equilibrium stability below 20% were excluded to ensure the formation of stable and specific duplexes. For final probe set selection, the finalized probe set, consisting of 28-32 probes per gene, was optimized to minimize spatial overlap, allowing a maximum of 5 nucleotides of overlap between adjacent probes. Probes were subjected to stringent filters for equilibrium, and free energy, to refine the probe library. Local BLAST searches within the probe pool identified and mitigated potential cross-hybridizations between the selected probes. Genes with insufficient probe numbers were curated manually using a genome browser to guarantee thorough coverage.

### CRISPRmap and RNAmap Readout Probe, CRISPRmap Padlock and Primer Oligo Design

For probe generation and off-target screening, starting with a base of 240,000 25-mer probes 8, we generated all possible 20-mer sequences sequentially. Each of these subsequences was subjected to BLAST screening against human and mouse transcriptomes to exclude any probes with off-target complementarity, and the resulting pool was thus reduced to only those probes with zero off-target hits. For optimizing Probe Performance: To optimize the readout probes' performance, we calculated their melting temperatures (Tm) and secondary structure predictions³⁹ to refine our selection further similar to the aforementioned target probes. This ensures that each probe binds to its intended target with high affinity and that the thermal profiles are suitable for our experimental conditions. In scenarios with high mRNA expression, it is vital to prevent overcrowding within any single fluorescence channel. Additionally, based on expression levels in the targeting tissue^{37,38}, we curated the probe sets and their corresponding fluorophores, and distributed the signal across multiple channels, promoting distinct visualization of each mRNA molecule. To further minimize the risk of cross-hybridization, we conducted an analysis of readout probe sequences for potential overlaps by performing a local BLAST search against the readout probe pool. This effort led to the identification of 226 20-mer DNA sequences, as outlined in Table 7, which provides details for each probe, including the 20-nt probe sequence, unique identifier, off-target information, melting temperatures (Tm), and secondary structures. For codebook construction: By employing a Hamming distance approach, similar to the HDM4 code used in MERFISH¹⁹, a codebook was constructed with 36 of the aforementioned 226 20-mer readout probes. This codebook consists of 319 36-bit codes allocated over 12 hybridization rounds across three channels (488 nm, 561 nm, 640 nm), ensuring that each readout probe would have a unique signature, reducing the possibility of channel crosstalk and fluorescence overlap. This approach aims to enable differentiation of probes even in densely labeled samples, where multiple mRNA molecules are in close proximity. The detailed codebook design is provided in Table 7, which includes details such as binary code assignments for each hybridization round and optical channel, indices, a conversion table that relates binary codes to specific probes, and sequences linked to each code across channels. For CRISPRmap readout probes, we selected 24 20-mers from the aforementioned 226 20-mer list. This selected set of 24 probes was split into 2 sets of 12 probes for the detection of Padlocks and Primers, respectively. Splints sequences consist of the reverse complement of the primer readout sequences, with an additional universal 2 bases added at the 5-prime end (“GT”), and the 3-prime end (“AC”) in an attempt to avoid ligation efficiency biases between different splint oligos. Two sets of 54 30-mer encoding sequences were generated with similar criteria as the 20mer list, and used as Padlock or Primer encoding sequences. The sequences of these oligos are listed in Table 7.

### Image Processing and Analysis:

### Image Storage and Stitching

All microscopy images were acquired using Fusion software and saved as ims files. Each ims file stores the image as a 5-D object in the order: Resolution, Channel, Z, Y, X. All image montages were stitched using Fusion's stitching software. For 60× images, high-speed setting was used to stitch the image montage and saved as ims files. For the 20× images the high-quality setting using default parameters was used to stitch the image montage and saved as ims files.

### Image Rescaling

All images were uploaded to a google cloud virtual machine for further image processing and analysis. To read the ims file and the corresponding metadata we used the “imaris_ims_file_reader” package. All 60× montages were analyzed at resolution 3 (⅛ scale of original image), and for all 20× images, we used resolution 1 images (½ scale of original image). All images were max projected along the Z-axis. All max projected images are 3 dimensional with the dimensions being Channel, Y, X. The images are in numpy array format and of uint16 data type. Our imaging protocol involves imaging cells at different magnifications based on the resolution of images required. If a particular imaging round was imaged at a different magnification, the images of this round will be of a different size and have a different pixel pitch (pixel to micron ratio). To accommodate for this, images were scaled to achieve a consistent pixel pitch using cv2 resize with a bicubic interpolation function. This also ensured that images from all imaging rounds were the same dimensions across X and Y.

### Image Registration

We register images to a reference image. Across the multiplexed imaging rounds there are global translational shifts, (i.e. misalignment of the glass bottom well plate), as well as local translational shifts (i.e. cells slightly shifting between imaging rounds). To finely align the images across all imaging rounds we calculated the transformation matrices for each round using the TV-L1²¹ implementation of optical flow on binary nuclei masks derived from DAPI stains. Optical flow calculates Y, X vector shifts across the images for every pixel and performs pixel level registration. The transformation matrix was then applied to all image channels of that imaging cycle. During registration all images are converted from uint16 to float64. The images are then converted back to uint16 to reduce the memory usage and speed up image processing. All registered images are 3 dimensional with the dimensions being Channel, Y, X. Registration quality was estimated using cross-correlation. It is expected that the cross-correlation would decrease with increasing montage size. For our 30×30 60× montages and 10×10 20× montages a cross-correlation of >0.75 was considered good.

### Segmentation of Cell and Nuclear Boundaries

To assign detected guides to each cell and quantify nuclear antibody stains, we segmented both the cytoplasmic area and nuclear area of each cell using Cellpose⁴¹. This process was broken into three steps, pre-processing, segmentation, and filtering. The EPCAM and DAPI stains were preprocessed by thresholding to maximize the dynamic range of the plasma membrane and nuclear stains in the base-editing screening. For other cultured cells, Wheat Germ Agglutinin (WGA)-CF770 (Biotium 29059) was used for membrane segmentation. For tissue sections, the membrane segmentation was performed on the E-cadherin staining. Typically, this involved setting pixels below the 2nd percentile to 0 and pixels above the 98th percentile to 255.

Following pre-processing, images were segmented twice with Cellpose, first to identify cytoplasmic areas and second to identify nuclear areas. The cytoplasmic segmentation run was performed on an image stack containing the EPCAM and DAPI stain and excluded any cytoplasm mask smaller than 5 pixels, while the nuclear segmentation was only performed on the DAPI stain with no minimum size requirement. The cell diameter parameter for Cellpose was determined by hand counting and averaging the width and height of 10 randomly sampled cells in pixels. This value was multiplied 1.5× for the cytoplasmic segmentation, and 0.75× for nuclear segmentation.

Once the nuclear and cytoplasmic masks were generated, we filtered out nuclear masks which did not overlap with a cytoplasmic mask, and cytoplasmic masks which did not contain a nuclear mask. This ensured that each segmented cytoplasm had one associated segmented nucleus and vice versa. The coordinates for each nuclear and cytoplasmic pair were relabeled with the nuclear ID, which was used as the cell ID from this point onwards. Segmentation quality was validated by both quantifying the percent of proposed cytoplasmic masks retained after filtering for segmented nuclei overlap and visual inspection of the images.

### CRISPRmap and RNAmap Amplicon Detection

To detect amplicons corresponding to CRISPRmap, all registered images corresponding to CRISPRmap readout rounds were processed as follows. Each 2D image (Y and X) underwent contrast stretching to improve the signal to noise ratio using skimage rescale_intensity function. Images were now stored in a list in the order of the readout round and channel (R1-ch1, R1-ch2, R1-ch3, R2-ch1 . . . ) with R1 being the first readout round and ch1 being the longest wavelength channel. For each image (readout round and channel combo) spots were identified by using the skimage implementation of the difference of gaussians method using parameters that maximized the barcode recovery. This implementation outputs an array of coordinates of all spots identified. All the coordinates for spots identified were searched against the cell masks (from cell segmentation) and any spot outside cell masks were discarded from further analysis. Furthermore, if the number of spots within a cell mask was <3 then the spots within the cell mask were also discarded from further analysis. This was done to reduce the noise/error in spot detection. All the spots retained for a given round-channel image were stored in an array. This was repeated for each cycle-channel image and the array of spots retained was stored in a list (with the order being spots for R1-ch1, R1-ch2, R1-ch3, R2-ch1 . . . ). Another array was created combining all the retained spots across all imaging round-channel combos. To eliminate duplicates in the merged array we used the np.unique function and discarded spots within a radius of 2 pixels. Then for each spot coordinate in the merged array, we compare the distance of the spot with all the spots detected in a single round-channel combo. If the spot is within a 2-pixel radius we mark the given round-channel combo as positive. This was done for all rounds and channels and by doing so for each spot a “spot code” was generated. A spot code essentially maps for a given spot, which round and channel combinations also contain that spot. Once the spot code is generated for all the spots, the spot code for every spot was compared to the predefined barcode designed for every guide. If a spot code matched a barcode, the spot was assigned to the barcode. If a spot did not map to any barcode corresponding to a guide, then the spot was discarded from further analysis. Spot calling was optimized to maximize spots that are assigned to barcodes of the guide library.

Finally, each cell was assigned a barcode based on the spot identity underneath the cell mask, according to the standard CRISPRmap QC as described above. The barcode identity of the cell was stored in a dictionary as well as in the format of an image mask.

### Foci and Micronuclei Detection, Cell Cycle Determination, and Quantification of Antibody Stains

For each cell, the sum intensity underneath a given cell mask was calculated for a given antibody stain and stored in a dictionary. The sum intensity underneath the nuclear mask was also calculated and stored in the dictionary. Also, the average intensity of each antibody stain was also calculated by dividing the sum intensity by the total number of pixels underneath the cell/nuclear mask. The raw images then underwent contrast stretching using the rescale_intensity function of skimage. After rescaling the images, foci detection for RAD51, BRCA1, RPA2, γH2AX, 53BP1 and RAD18 was done using skimage difference of gaussians method. The total number of foci within the cell mask/nuclear mask was also stored in the dictionary.

To detect and quantify the presence of micronuclei, for every cell mask in the image, the nuclear mask was retrieved. If there was a nuclear mask of <100 pixels in area, then the nuclear mask was separately annotated as a small nucleus.

For micronuclei detection, we first perform nuclei segmentation on the DAPI stain using Cellpose. Nuclei masks were generated to define the outline of each nucleus in the image. All the nuclei with a size less than 100 pixels were marked as “micronuclei”. This threshold was determined by manually inspecting the micronuclei captured by the nuclei segmentation. We then subtract the DAPI signal underneath all nuclei masks by changing the intensity value to 0 for all pixels outlined by the nuclei masks. We remove the background DAPI signal by changing the intensity value to 0 for any pixel with an intensity value below 110. To completely remove the residual DAPI signal coming from the DAPI staining of the cell nuclei, we dilate each nuclei mask by 2 pixels using cv2 then changing the intensity value to 0 for all pixels outlined by the dilated nuclei masks. We finally perform spot calling to identify micronuclei. Based on the coordinates of the spots, the number of spots within a cell mask were identified and were included in the dictionary as the number of micronuclei within a cell mask.

Multiplexed immunostaining on cell cycle phase marker proteins was performed to distinguish cells in different cell cycle phases (i.e. G0, G1, S/G2, and M phases). Three antibodies were used for cell cycle phases classification: 0.5 μg/ml Rabbit monoclonal anti-Ki-67 (Cell Signaling Technology 34330), 1 μg/ml Rabbit monoclonal anti-Cyclin A2 (Cell Signaling Technology 29113SF), 0.5 μg/ml Rabbit monoclonal anti-phospho-Histone H3 (Cell Signaling Technology 3475). 1 μg/ml Rabbit monoclonal anti-Cyclin B1 (Cell Signaling Technology 65173SF) was included to further validate the specificity of the cyclin A2 staining. Briefly, cell cycle marker signals were quantified for each cell by calculating the average fluorescence intensity under the nuclear mask. The distribution of the average nuclear intensity of each marker was plotted and a threshold was set to divide cells into positive or negative populations for that marker (shown in FIG. 28G). First, cells in the Ki-67 negative population were classified as G0 phase cells, and cells positive in phospho-Histone H3 as M phase cells, then cells in cyclin A2 positive population were classified as S/G2 phase cells and the remaining cells were classified as G1 phase cells.

### Statistical Analysis on Foci Features in the Base-Editing Screen

For each foci feature we acquired under each treatment condition in the base-editing screen, we calculated a p-value based on the Kolmogorov-Smirnov test (K-S test) between the foci count distribution in cells assigned with a given guide identity and all cells assigned with AAVS1-targeting or Non-targeting control guide identities (control cells), then calculated the log 2-foldchange (L2FC) between the average foci count in cells assigned with a given guide identity and the average foci count in all control cells. The adjusted p-value (p.adj) was calculated by the Benjamini & Hochberg method. Statistical significance in foci number change is defined by p.adj<0.05 and absolute L2FC >0.5. Volcano plots were generated to visualize the L2FC and p.adj distribution for a given foci feature under a given treatment (FIGS. 20G-H, FIGS. 20J-K, FIGS. 30G-H, FIGS. 30J-K). We evaluated the foci optical features we acquired over the ionizing radiation and DNA damaging agents treatments for each gene variant in the library and counted in how many features a variant has resulted in statistically significant change compared to control cells. We tested the number of significant optical features scored by variants in different ClinVar categories with a two-sided Mann-Whitney test (FIG. 30M). We also investigated the guides in our library that can lead to the same intended amino acid (AA) change. We compared the Pearson correlation of L2FC across all foci features in guides leading to same AA changes and guides leading to different AA changes with a two-sided independent t-test (FIG. 30N).

### Sample Size Analysis for Base-Editing Screen Hits

In the DDR364 library, we included 72 control (non-targeting or AAVS1-targeting) guides. In the irradiation dataset, we profiled 20,029 control cells over these 72 control guides, whereas the median cell number of a perturbation guide is 281 cells. Therefore, the control population size is roughly 72-fold of a guide on average. In S/G2-phase cells, the median number of cells with perturbation guides is 99 cells. Here, we selected S/G2-phase cells from the top 4 screen hits of RAD51 foci and BRCA1 foci, respectively, to perform the random sampling analysis. Given the fact that if we vary the number of cells we image, the size of the control population will still be roughly 72-fold of a given guide, so we performed random sampling on the control cells accordingly to maintain this ratio. For each guide, we randomly sampled with replacement for n=20, 40, . . . , 200 cells whereas we sampled n*72 cells from the control cells in S/G2 phase, and the p-value is calculated by the K-S test, with statistical significance determined by p<0.05. The result is shown in FIG. 31B & FIG. 31D.

### Statistical Analysis on Colocalized Foci Features for Base-Editing Screen

All colocalization analysis was performed on the Irradiated (IR) dataset using images at Resolution 1 (½ scale of the original image), and foci for the 6 antibody stains (BRCA1, RAD51, γH2AX, 53BP1, RAD18 and RPA2) were detected using skimage blob_dog method. To account for minor shifts that may not be corrected by registration, foci are considered to colocalize with other foci if the centroids were within a 4 pixel euclidean distance. This was performed for all 15 (6 choose 2) pairwise combinations of the six antibody stains, and the number of colocalized foci for each pair was then calculated for each single cell that has been assigned with a guide identity (i.e. passed QC) by mapping the colocalized foci to its cell nucleus mask.

To determine if the number of colocalized foci observed within the nucleus could be by random overlap of 2 foci markers, we fixed the coordinates of the first foci marker and permuted the foci for the second marker 10,000 times inside the nucleus. Each time we calculated the number of colocalized foci using the method described above. The results are shown in FIG. 38A. To determine if there was any relevance of cell cycle on the colocalization of foci we calculated the differences in abundance between a colocalized foci at the S/G2 phase and G1 phase. The results are shown in FIG. 38B. Finally, to calculate the proportion of any given foci co-localizing with another foci, we calculated the proportion of foci colocalized using the formula mean (Number of colocalized foci A-B/min(Number of colocalized foci A, Number of colocalized foci B) per cell). The results are shown in FIG. 38C & FIG. 38D.

### Void Analysis of Tissue Images

Voids were identified by finding the outermost low intensity contours where the E-Cadherin stain intensity was equal to 113. To set a minimum size for voids, contours with more than 100 boundary pixels were retained as tissue voids. For each retained contour we gathered the intensity values for the anti-mouse Cd31 stain and the anti-human cleaved PARP1 stain within 20 pixels of the edge of the contour (equidistant inside and outside), which we will term boundary stains. We calculated the 90th percentile of the Cd31 and cPARP boundary stains, and classified voids as mouse vasculature if Cd31 value was above 113, or as cell death if the cPARP value was above 104. Voids negative in both were left unclassified, and one void was classified as double positive.

### Clonal Purity Calculations on Tissue Images

Following recently reported clonality analysis of barcoded cells²⁷, the clonal score was determined in a cell-centric manner, by calculating the local clustering coefficient. Briefly, we constructed a 10 nearest neighbors graph for each cell, and assigned a P Value to each neighborhood by comparing the cell's same-guide clustering coefficient to a table of homotypic clustering coefficients from randomly arranged neighborhoods. This P-value was corrected using the Bonferroni Correction. Cells with a P adjusted <0.05 were then plotted along with the same guide cells in the 10 nearest neighbor graph to identify clonal regions with significantly higher clustering coefficient.

### Bootstrapped Wasserstein Distance

We computed a bootstrapped Wasserstein distance to measure the foci expression deviation from perturbation to control guides. We denote X_{{g},j}∈Z^{|g|} and X_{{c},j}∈Z^{|c|} as cells undergoing a specific perturbation or control respectively, where |g|, |c| refer to the corresponding number of cells in each condition. For each perturbation gᵢ∈[[G]] and feature j∈(RAD51, BRCA1, RPA2, γH2AX, X53BP1 and RAD18), we computed

$$W ( {g}_{i} , j ) = \frac{1}{N} \cdot {\sum }_{n = 1}^{N} {W}_{1} ( {X}_{\{ {g}_{i}^{( 1 )} , ... , {g}_{i}^{( S )} \} , j} , {X}_{\{ c \} , j} )$$

as the average 1-Wasserstein distance between guide i and control across S=50 samples with N=200 iterations, where in each iteration S, cells{gᵢ⁽¹⁾, . . . , gᵢ^{(S)}}⊆{gᵢ} under guide i are randomly sampled without replacement. As a baseline control, we also computed

$$W ( c , j ) = \frac{1}{N} \cdot {\sum }_{n = 1}^{N} {W}_{1} ( {X}_{\{ {c}^{( 1 )} , ... {c}^{( S )} \} , j} , {X}_{\{ c \} , j} )$$

as the average 1-Wasserstein distance between randomly subsampled control cells and the full control cell set. The choice of bootstrapping cells is to mitigate the bias introduced by noticeable sample size differences across guides. We report the bootstrapped distances across perturbation guides and the control baseline with violin plots (FIGS. 25J-K). Highlighted guides are chosen from the aforementioned significant hits with absolute log 2-fold change (L2FC) >0.5 and KS-test p-value (p.adj)<0.05 under Benjamini-Hochberg correction.

### Beta-Binomial Test

We define the data as X∈Z^{N×2}, where Xᵢ:=(nᵢ, kᵢ), where nᵢ denotes the number of cells affected by a given guide, gᵢ, and kᵢ denotes the number of cells exceeding a fixed threshold for a cell-specific continuous or discrete feature. We refer to these cells as “positive” cells. Each guide, gᵢ ∈[[G]], corresponds to either a control guide or a test guide defined by a mapping φ(gᵢ):[[G]]→{0, 1}, where φ(gᵢ)=1 corresponds to a test guide. To construct a plausible null hypothesis, we fit a Beta-Binomial distribution to the control data, X_{ctrl}:={Xᵢ:φ(gᵢ)=0}. We use a Beta-Binomial distribution since we assume the number of positive cells is independently and identically distributed according to a Binomial distribution given the number of total cells for a guide. To account for overdispersion attributed to variability between control guides, we place a Beta distribution on the success probability of the Binomial distribution. For each condition the cells are placed in, we run a separate statistical test since we assume the rate of positive cells is significantly impacted by the environment, and we would like to test for the significance of specific guides conditioned on the environment.

The model is as follows:
pᵢ˜Beta(α, β)
kᵢ˜Binomial(nᵢ, pᵢ),
where the parameters α, β are inferred via a maximum likelihood estimation. Then, over the test data, Xₜₑₛₜ:={Xᵢ:φ(gᵢ)=1}, we compute p-values according to a two-tailed test. These p-values are then adjusted using the Benjamini-Hochberg correction procedure to control for the false discovery rate (FDR). Finally, we reject the null for any corrected p-values falling below the significance level of 0.05. To ensure the null hypothesis is plausible with respect to the control data, we check to see if the computed p-values are uniformly distributed over the [0, 1] interval. The proportions, total number of cells, and adjusted p-values for each test guide are reported in Table 7.

### Statistics and Reproducibility

Histograms were plotted with the histplot function, boxplots with the boxplot function, scatter plots and volcano plots with the scatterplot function, ECDF plots with the ecdfplot function, and violin plots with the violinplot function in the seaborn package (0.11.1) in python. Two-sided Mann Whitney U tests and student t-tests were performed to test the difference between the distributions using the statannotations package in python. *p<0.05, **p<0.01, ***p<0.001, ***p<0.0001. The difference in foci number distribution was tested by a two-sided Kolmogorov-Smirnov test using the ks.test function in R and the p values were adjusted by the Benjamini-Hochberg method. *p.adj<0.05, **p.adj<0.01, ***p.adj<0.001, ****p.adj<0.0001. Guides with p.adj<0.05 and an absolute L2FC >0.5 were regarded as statistically significant. Fisher exact test for gene enrichment was performed with the fisher.test function in R and the p values were adjusted by the Benjamini-Hochberg method. Genes with an adjusted p-value <0.05 were regarded as statistically significant. Heatmap for optical feature correlations and hierarchical clustering of guides were generated with the clustermap function in the seaborn package (0.11.1) in python, method “complete” was used for clustering. Schematics were generated in BioRender. All representative images shown in the manuscript were repeated in at least three technical replicates with similar results, unless otherwise specified in the figure legends.

### Table Information

Table 1. Cost estimation of the CRISPRmap assay. The cost of probes and fluorophores, key chemicals and reagents, and enzymatic reactions are listed. The calculation of cost per million cells is based on the base-editing screening on MCF7 cells using the DDR364 library. The actual cost may differ according to price changes on the vendors' side. The total setup cost mainly results from the purchase of Padlock, primer, splint and readout probes, which require a minimum amount to be ordered. Cost of Conventional OPS (Feldman et al., 2019) is included for side-by-side comparison with CRISPRmap on a per six-well plate basis.

Table 2. GFP-pilot analysis. This table shows the single-cell level data for cells analyzed in the CRISPRmap knockout screening targeting GFP, related to FIG. 18 and FIG. 23. The number of amplicons detected as well as the average nuclear GFP intensity in each cell are listed. Single-cell level data for the non-infection control in the same experiment is included for the evaluation of non-specific amplicon generation. Cells that passed QC are listed with their unique guide identities. The pairwise Mann-Whitney U test p values of each GFP-NTC guide pair are listed, related to FIG. 23A. Sensitivity, specificity, precision and the ratio of cells passed quality check (QC) on HT 1080s in different experimental settings are listed, related to FIG. 23B. And the sensitivity, specificity, precision and ratio of cells passedQC under 25 QC metrics are listed, related to FIGS. 23C-E. Number of reads classified into each category in the recombination analysis are listed for the GFPpilot and DDR364 library at the Opool, Plasmid, and gDNA level, respectively, related to FIG. 23J. The ratio of cells with unique or double barcode detection are listed, side-by-side with the Poisson expected double transduction rate, for the cells transduced with the GFP-pilot library at different multiplicity of infection (MOI), related to FIG. 23L. The total number of cells profiled and the fraction of cells with barcode detection in the CRISPRmap and Conventional OPS assays performed on different cell types are listed, related to FIG. 18F.

Table 3. Base-editing screening data analysis. Information of the guides in the DDR364 library are listed in detail, including the sgRNA sequences, type of intended mutations, target genes, intended Amino Acid change, updated Clinvar categories. The number of RNAmap spots and TPM RNA-seq reads for the 12 transcripts are listed, related to FIG. 19E. The KS test p values, FDR, and log 2-fold change (LFC) of each guide on each foci features in the irradiation (irradiation) and DNA damaging agents (chemo) screening are shown, the sgRNA_IDs, Rule Set 2 on-target scores, sgRNA categories and Clinvar categories are listed alongside, related to FIG. 20, FIG. 21, FIG. 30, and FIGS. 35-37. P values of gene enrichment fisher test are listed, related to FIG. 20I & FIG. 20L and FIG. 30I & FIG. 30L. Percentage library representation of guides based on cells assigned with guide-reporting barcodes in seven conditions in the base-editing screen and based on reads sequenced at the plasmid library level are shown, related to FIG. 29. The wasserstein distances for all foci features are shown, related to FIG. 30A & FIG. 30B. The p values of beta binomial tests for all binary features are shown, related to FIG. 30C. The FDR and LFC of foci features for the 9 individually transduced guides are shown, related to FIG. 33. Base-editing efficiency for the 3 hits identified in the IR screen and 2 AAVS1 control guides are listed for in-window and out-of-window C bases, related to FIG. 34A.

Table 4. OE19 tissue analysis. Cells analyzed in the OE19 tissue profiling shown in FIG. 22 are listed at the single-cell level for the evaluation of spot count, guide purity, apoptotic marker expression and cell sizes. The library representation in three tissue samples and relative abundance of guides in plasmid library sequencing reads are listed, related to FIG. 39B & FIG. 39C. The estimated tissue area required for three different library sizes based on three tissue samples are shown, related to FIG. 39D & FIG. 39E.

Table 5. GFP-pilot library design and readout scheme. This table lists the sgRNA_ID, sgRNA sequences and barcode sequences in the GFP-pilot library, as well as the sequences of padlocks, primers, splints and readout probes that were used to readout the barcodes, related to FIG. 18 and FIG. 23. A detailed readout scheme including the order, conjugated fluorophores and imaging setting of the 8 readout probes are listed, as well as the 8-bit codebook encoding the 10 guides in the library. The oligonucleotide sequences used in the GFP-mTurquoise2 barcode detection experiment are also listed.

Table 6. DDR364 library design, readout scheme, and antibody staining. This table lists the sgRNA_ID, sgRNA sequences and barcode sequences in the DDR364 library, related to FIGS. 19, 20, 21 and FIGS. 28-37. The sequences of padlocks, primers, splints and readout probes that were used to readout the barcodes and a detailed readout scheme including the order, conjugated fluorophores and imaging setting of the 24 readout probes are shown, together with the 24-bit codebook encoding the 364 guides in the library. The mutational outcomes and ClinVar annotations of the guides are listed. Antibodies used in the irradiation screen, DNA damaging agents screen (chemo) are listed in detail. The antibodies used in OE19 tissue profiling are also listed in detail, related to FIG. 22 and FIG. 39.

Table 7. Oligonucleotide sequences of CRISPRmap probes. This table lists the sequence of all the 54 padlocks and 54 primers used in the DDR364 base-editing screening. All 2,916 possible padlock-primer combinations are listed with their corresponding readout sequences specified. This table also lists the sequences of 12 splints and 24 readout probes that are required for the barcode detection of the 2,916 combinations. Sequences of PCR primers used for library amplification and validation are listed. For RNAmap, the sequences of 36 readout probes and their corresponding 319 36-bit codes are listed, as well as the readout scheme and the codebook for the 12 selected transcripts profiled in the DDR364 irradiation screen. Primer sequences for PCR amplification of the genomic loci of the intended mutations are listed for the five individually transduced guides in the base-editing efficiency validation.

Table 8. Cells passed QC in the irradiation base-editing screening and the optical features. This table lists 226,369 cells in the irradiation base-editing screening that passed the QC criteria, related to FIG. 19 and FIG. 30. The average nuclear intensity of protein stains, cell cycle phase annotation, the raw count for foci features, and the number of RNA-reporting spots measured by RNAmap for the 12 transcripts are listed. Cells are then grouped by sgRNA_ID for FDR and LFC calculation listed in Table 3.

In Example 2, Table 1 refers to Supplementary Table 1 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 2 refers to Supplementary Table 2 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 3 refers to Supplementary Table 3 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 4 refers to Supplementary Table 4 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 5 refers to Supplementary Table 5 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 6 refers to Supplementary Table 6 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 7 refers to Supplementary Table 7 of Gu, J. et al. “Mapping multimodal phenotypes to perturbations in cells and tissue with CRISPRmap.” Nat Biotechnol 43, 1101-1115 (2025) and the contents of which are hereby incorporated by reference. Table 8 refers to Supplementary Table 8 of Gu, J. et al. “Mapping multimodal phenotypes to perturbations in cells and tissue with CRISPRmap.” Nat Biotechnol 43, 1101-1115 (2025) and the contents of which are hereby incorporated by reference. The entire contents of Gu, J. et al. “Mapping multimodal phenotypes to perturbations in cells and tissue with CRISPRmap.” Nat Biotechnol 43, 1101-1115 (2025) are hereby incorporated by reference.

### REFERENCES

1. Datlinger, P. et al. Pooled CRISPR screening with single-cell transcriptome readout. Nat Methods 14, 297-301 (2017).
2. Dixit, A. et al. Perturb-Seq: Dissecting Molecular Circuits with Scalable Single-Cell RNA Profiling of Pooled Genetic Screens. Cell 167, 1853-1866 e1817 (2016).
3. Feldman, D. et al. Pooled genetic perturbation screens with image-based phenotypes. Nat Protoc 17, 476-512 (2022).
4. Feldman, D. et al. Optical Pooled Screens in Human Cells. Cell 179, 787-799 e717 (2019).
5. Funk, L. et al. The phenotypic landscape of essential human genes. Cell 185, 4634-4653 e4622 (2022).
6. Wang, C., Lu, T., Emanuel, G., Babcock, H. P. & Zhuang, X. Imaging-based pooled CRISPR screening reveals regulators of lncRNA localization. Proc Natl Acad Sci USA 116, 10842-10851 (2019).
7. Cuella-Martin, R. et al. Functional interrogation of DNA damage response variants with base editing screens. Cell 184, 1081-1097 e1019 (2021).
8. Hanna, R. E. et al. Massively parallel assessment of human variants with base editor screens. Cell 184, 1064-1080 e1020 (2021).
9. Anzalone, A. V., Koblan, L. W. & Liu, D. R. Genome editing with CRISPR-Cas nucleases, base editors, transposases and prime editors. Nat Biotechnol 38, 824-844 (2020).
10. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
11. Matejcic, M. et al. Pathogenic Variants in Cancer Predisposition Genes and Prostate Cancer Risk in Men of African Ancestry. JCO Precis Oncol 4, 32-43 (2020).
12. Ciccia, A. & Elledge, S. J. The DNA damage response: making it safe to play with knives. Mol Cell 40, 179-204 (2010).
13. Liu, L. F. et al. Mechanism of action of camptothecin. Ann NY Acad Sci 922, 1-10 (2000).
14. Goulooze, S. C., Cohen, A. F. & Rissmann, R. Olaparib. Br J Clin Pharmacol 81, 171-173 (2016).
15. Dasari, S. & Tchounwou, P. B. Cisplatin in cancer therapy: molecular mechanisms of action. Eur J Pharmacol 740, 364-378 (2014).
16. Montecucco, A., Zanetta, F. & Biamonti, G. Molecular mechanisms of etoposide. EXCLIJ 14, 95-108 (2015).
17. Dhainaut, M. et al. Spatial CRISPR genomics identifies regulators of the tumor microenvironment. Cell 185, 1223-1239 e1220 (2022).
18. Wang, X. et al. Three-dimensional intact-tissue sequencing of single-cell transcriptional states. Science 361 (2018).
19. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
20. Shah, S. et al. Dynamics and Spatial Genomics of the Nascent Transcriptome by Intron seqFISH. Cell 174, 363-376 e316 (2018).
21. Wedel, A., Pock, T., Zach, C., Bischof, H. & Cremers, D. An Improved Algorithm for TV-L1 Optical Flow, Edn. Statistical and Geometrical Approaches to Visual Motion Analysis: International Dagstuhl Seminar, Dagstuhl Castle. (Springer Berlin Heidelberg, Berlin, Heidelberg; 2009).
22. Radtke, A. J. et al. IBEX: A versatile multiplex optical imaging approach for deep phenotyping and spatial analysis of cells in complex tissues. Proc Natl Acad Sci USA 117, 33455-33465 (2020).
23. McManus, K. J. & Hendzel, M. J. ATM-dependent DNA damage-independent mitotic phosphorylation of H2AX in normally growing mammalian cells. Mol Biol Cell 16, 5013-5025 (2005).
24. Landrum, M. J. et al. ClinVar: improvements to accessing data. Nucleic Acids Res 48, D835-D844 (2020).
25. Doench, J. G. et al. Optimized sgRNA design to maximize activity and minimize off-target effects of CRISPR-Cas9. Nat Biotechnol 34, 184-191 (2016).
26. Kweon, J. et al. A CRISPR-based base-editing screen for the functional assessment of BRCA1 variants. Oncogene 39, 30-35 (2020).
27. Rovira-Clave, X. et al. Spatial epitope barcoding reveals clonal tumor patch behaviors. Cancer Cell 40, 1423-1439 e1411 (2022).
28. Eng, C. L. et al. Transcriptome-scale super-resolved imaging in tissues by RNA seqFISH. Nature 568, 235-239 (2019).
29. Carlson, R. J., Leiken, M. D., Guna, A., Hacohen, N. & Blainey, P. C. A genome-wide optical pooled screen reveals regulators of cellular antiviral responses. Proc Natl Acad Sci USA 120, e2210623120 (2023).
30. Askary, A. et al. In situ readout of DNA barcodes and single base edits facilitated by in vitro transcription. Nat Biotechnol 38, 66-75 (2020).
31. Pantazis, C. B. et al. A reference human induced pluripotent stem cell line for large-scale collaborative studies. Cell Stem Cell 29, 1685-1702 e1622 (2022).
32. Bos, P. H. et al. Development of MAP4 Kinase Inhibitors as Motor Neuron-Protecting Agents. Cell Chem Biol 26, 1703-1715 e1737 (2019).
33. Maury, Y. et al. Combinatorial analysis of developmental cues efficiently converts human pluripotent stem cells into multiple neuronal subtypes. Nat Biotechnol 33, 89-96 (2015).
34. CRISPick (the Broad Institute of MIT and Harvard.
35. Omelina, E. S., Ivankin, A. V., Letiagina, A. E. & Pindyurin, A. V. Optimized PCR conditions minimizing the formation of chimeric DNA molecules from MPRA plasmid libraries. BMC Genomics 20, 536 (2019).
36. Eng, C. L., Shah, S., Thomassie, J. & Cai, L. Profiling the transcriptome with RNA SPOTs. Nat Methods 14, 1153-1155 (2017).
37. Consortium, G. T. The GTEx Consortium atlas of genetic regulatory effects across human tissues. Science 369, 1318-1330 (2020).
38. Mouse, E. C. et al. An encyclopedia of mouse DNA elements (Mouse ENCODE). Genome Biol 13, 418 (2012).
39. Altschul, S. F., Gish, W., Miller, W., Myers, E. W. & Lipman, D. J. Basic local alignment search tool. J Mol Biol 215, 403-410 (1990).
40. Zadeh, J. N. et al. NUPACK: Analysis and design of nucleic acid systems. J Comput Chem 32, 170-173 (2011).
41. Stringer, C., Wang, T., Michaelos, M. & Pachitariu, M. Cellpose: a generalist algorithm for cellular segmentation. Nat Methods 18, 100-106 (2021).

What is claimed is:

1. A composition comprising a barcoding oligonucleotide pair, the pair comprising:
a) a single-stranded DNA padlock detection oligonucleotide molecule comprising
i) a first primer detection oligonucleotide hybridization portion;
ii) a padlock barcode portion (H1) comprising a nucleotide sequence complementary to a first targeted nucleotide sequence (H1′) of a targeted polynucleotide molecule;
iii) a first padlock readout portion (rs2);
iv) a second padlock readout portion (rs1); and
v) a second primer detection oligonucleotide hybridization portion; and
b) a single-stranded DNA primer detection oligonucleotide molecule comprising
i) a primer barcode portion (H2) comprising a nucleotide sequence complementary to a second targeted nucleotide sequence (H2′) of the targeted polynucleotide molecule;
ii) a first padlock detection oligonucleotide hybridization portion comprising a nucleotide sequence complementary to the first primer detection oligonucleotide hybridization portion nucleotide sequence;
iii) a first primer readout portion (rs4);
iv) a second primer readout portion (rs3); and
v) a second padlock detection oligonucleotide hybridization portion comprising a nucleotide sequence complementary to the second primer detection oligonucleotide hybridization portion nucleotide sequence.

2. The composition of claim 1, further comprising a first single-stranded DNA oligonucleotide splint molecule comprising a nucleotide sequence complementary to the first primer readout portion (rs4) nucleotide sequence.

3. The composition of claim 1, further comprising a second single-stranded DNA oligonucleotide splint molecule comprising a nucleotide sequence complementary to the first primer readout portion (rs3) nucleotide sequence.

4. The composition of claim 1, comprising one to four single-stranded DNA oligonucleotide probe molecules,
wherein a probe molecule of the one to four single-stranded DNA oligonucleotide probe molecules is conjugated to a detection molecule and comprises an amplicon hybridization portion;
wherein the amplicon hybridization portion of a probe molecule of the one to four single-stranded DNA oligonucleotide probe molecules consists of
i) a nucleotide sequence complementary to the first padlock readout portion (rs2) nucleotide sequence;
ii) a nucleotide sequence complementary to the second padlock readout portion (rs1) nucleotide sequence;
iii) the first primer readout portion (rs4) nucleotide sequence; or
iv) the second primer readout portion (rs3) nucleotide sequence.

5. A method of detecting a polynucleotide molecule in a cell, the method comprising:
a) delivering to the cell the composition according to claim 1;
b) hybridizing the padlock detection molecule and the primer detection molecule to the polynucleotide molecule, and hybridizing the padlock detection molecule to the primer detection molecule;
c) delivering to the cell
i) a first single-stranded DNA oligonucleotide splint molecule comprising a nucleotide sequence complementary to the first primer readout portion (rs4) nucleotide sequence; and
ii) a second single-stranded DNA oligonucleotide splint molecule comprising a nucleotide sequence complementary to the first primer readout portion (rs3) nucleotide sequence;
d) hybridizing the first splint molecule to the primer detection molecule and hybridizing the second splint molecule to the primer detection molecule;
e) ligating the first and second splint molecules to the hybridized padlock detection molecule to form a circularized amplicon template oligonucleotide molecule;
f) amplifying the circularized amplicon template oligonucleotide molecule by rolling circle amplification to form an amplicon molecule;
g) delivering to the cell one to four single-stranded DNA oligonucleotide probe molecules,
wherein each probe molecule of the one to four single-stranded DNA oligonucleotide probe molecules is conjugated to a detection molecule and comprises an amplicon hybridization portion;
wherein the amplicon hybridization portion comprises a sequence selected from
i) a nucleotide sequence complementary to the first padlock readout portion (rs2) nucleotide sequence;
ii) a nucleotide sequence complementary to the second padlock readout portion (rs1) nucleotide sequence;
iii) the first primer readout portion (rs4) nucleotide sequence; or
iv) the second primer readout portion (rs3) nucleotide sequence; and
h) optically detecting the presence of the polynucleotide molecule in the cell upon hybridization of a probe molecule to the amplicon molecule, preferably optically detecting the presence of the polynucleotide molecule in the cell upon hybridization of each probe molecule delivered to the cell to the amplicon molecule.

6. A cellular barcoding library, the library comprising a plurality of barcoding oligonucleotide pairs according to claim 1, wherein each barcoding oligonucleotide pair comprises a combined readout set of rs1, rs2, rs3 and rs4 sequences; and wherein each combined readout set is unique to its barcoding oligonucleotide pair within the library.

7. A composition comprising a transcript profiling oligonucleotide pair, the pair comprising:
a) a single-stranded DNA padlock detection oligonucleotide molecule comprising
i) a first primer detection oligonucleotide hybridization portion;
ii) a padlock transcript-binding portion (H1) comprising a nucleotide sequence complementary to a first targeted nucleotide sequence (H1′) of a targeted polynucleotide molecule;
iii) a first padlock readout portion (rs1);
iv) a second padlock readout portion (rs2);
v) a third padlock readout portion (rs3);
vi) a second primer detection oligonucleotide hybridization portion; and
b) a single-stranded DNA primer detection oligonucleotide molecule comprising
i) a primer transcript-binding portion (H2) comprising a nucleotide sequence complementary to a second targeted nucleotide sequence (H2′) of the targeted polynucleotide molecule;
ii) a first padlock detection oligonucleotide hybridization portion comprising a nucleotide sequence complementary to the first primer detection oligonucleotide hybridization portion nucleotide sequence;
iii) a splint hybridization primer readout portion (rs4); and
iv) a second padlock detection oligonucleotide hybridization portion comprising a nucleotide sequence complementary to the second primer detection oligonucleotide hybridization portion nucleotide sequence.

8. The composition of claim 7, wherein the splint hybridization primer readout portion (rs4) comprises a central splint hybridization portion and is flanked by additional nucleotides on each end, preferably wherein the central hybridization portion is about 20 nucleotides in length and is flanked by about 2 nucleotides on each end.

9. The composition of claim 7, further comprising a single-stranded DNA oligonucleotide splint molecule comprising a nucleotide sequence complementary to the rs4 nucleotide sequence.

10. The composition of claim 7, further comprising one to four single-stranded DNA oligonucleotide probe molecules,
wherein a probe molecule of the one to four single-stranded DNA oligonucleotide probe molecules is conjugated to a detection molecule and comprises an amplicon hybridization portion;
wherein the amplicon hybridization portion of a probe molecule of the one to four single-stranded DNA oligonucleotide probe molecules consists of
i) a nucleotide sequence complementary to the first padlock readout portion (rs2) nucleotide sequence;
ii) a nucleotide sequence complementary to the second padlock readout portion (rs1) nucleotide sequence;
iii) the rs4 nucleotide sequence; or
iv) the rs3 nucleotide sequence.

11. A transcript profiling library comprising a plurality of transcript profiling oligonucleotide pairs according to claim 7, wherein any targeted polynucleotide molecule is capable of being hybridized by 3-8 transcript profiling oligonucleotide pairs within the library and/or wherein the targeted polynucleotide molecule is an RNA molecule, preferably an mRNA molecule.

12. A cellular barcoding library, the library comprising a plurality of barcoding oligonucleotide pairs according to claim 7,
wherein each barcoding oligonucleotide pair comprises a combined readout set of rs1, rs2, rs3 and rs4 sequences;
wherein each combined readout set is unique to its barcoding oligonucleotide pair within the library.

13. The cellular barcoding library of claim 12, wherein
each first primer detection oligonucleotide hybridization portion is a universal sequence common to each padlock detection oligonucleotide molecule;
each second primer detection oligonucleotide hybridization portion is a universal sequence common to each padlock detection oligonucleotide molecule;
each first padlock detection oligonucleotide hybridization portion is a universal sequence common to each primer detection oligonucleotide molecule; and/or
each second padlock detection oligonucleotide hybridization portion is a universal sequence common to each primer detection oligonucleotide molecule.

14. The cellular barcoding library of claim 12, wherein each targeted molecule in a library of targeted molecules comprises a unique targeted portion comprising a first targeted nucleotide sequence (H1′) and a second targeted nucleotide sequence (H2′),
a) wherein the first targeted nucleotide sequence (H1′) is complementary to a padlock barcode portion (H1) of a padlock detection molecule of a barcoding oligonucleotide pair in the library; and
b) wherein the second targeted nucleotide sequence (H2′) is complementary to the primer barcode portion (H2) of a primer detection molecule of the said barcoding oligonucleotide pair in (a);
such that no two targeted molecules in the library of targeted molecules share the same unique targeted portion.

15. The cellular barcoding library of claim 12, further comprising a plurality of single-stranded DNA oligonucleotide splint molecules, wherein each splint molecule
comprises a nucleotide sequence complementary to a rs4 nucleotide sequence of a primer detection oligonucleotide molecule in the cellular barcoding library.

16. The cellular barcoding library of claim 12, further comprising a plurality of DNA oligonucleotide probe molecules,
wherein each probe molecule is conjugated to a detection molecule and comprises an amplicon hybridization portion;
wherein the amplicon hybridization portion comprises
i) a nucleotide sequence complementary to a first padlock readout portion (rs2) nucleotide sequence of a padlock detection molecule of the cellular barcoding library;
ii) a nucleotide sequence complementary to a second padlock readout portion (rs1) nucleotide sequence of a padlock detection molecule of the cellular barcoding library;
iii) a rs4 nucleotide sequence of a primer detection molecule of the cellular barcoding library; or
iv) a rs3 nucleotide sequence of a padlock detection molecule of the cellular barcoding library.

17. A method of detecting a polynucleotide molecule from a library of targeted molecules in a cell, the method comprising
a) delivering a library of targeted molecules to a plurality of cells, preferably such that each cell in the plurality is delivered a targeted molecule;
b) delivering the cellular barcoding library of claim 12;
c) hybridizing the padlock detection molecule and the primer detection molecule of each barcoded oligonucleotide pair to a targeted polynucleotide molecule present in a cell, and hybridizing the padlock detection molecule to the primer detection molecule of each barcoded oligonucleotide pair;
d) delivering the plurality of splint molecules comprises a nucleotide sequence complementary to a rs4 nucleotide sequence of a primer detection oligonucleotide molecule in the cellular barcoding library;
e) ligating a splint molecule to a hybridized padlock detection molecule to form circularized amplicon template oligonucleotide molecules;
f) amplifying each circularized amplicon template oligonucleotide molecule by rolling circle amplification to form amplicon molecules;
g) delivering a first group of probe molecules from the plurality of single-stranded DNA oligonucleotide probe molecules, wherein each probe molecule is conjugated to a detection molecule and comprises an amplicon hybridization portion, wherein the amplicon hybridization portion comprises a nucleotide sequence complementary to a first padlock readout portion (rs2) nucleotide sequence of a padlock detection molecule of the cellular barcoding library; a nucleotide sequence complementary to a second padlock readout portion (rs1) nucleotide sequence of a padlock detection molecule of the cellular barcoding library; a rs4 nucleotide sequence of a primer detection molecule of the cellular barcoding library; or a rs3 nucleotide sequence of a padlock detection molecule of the cellular barcoding library,
wherein each probe molecule in the group comprises a unique amplicon hybridization portion which is different in sequence from the other probe molecules in the group;
h) optically detecting the hybridization of the probe molecules from the first group to any amplicon molecule in any cell of the plurality of cells;
i) for each amplicon location,
mark “1” for each channel in which a hybridization of a probe molecule to the amplicon is observed; and
mark “0” for each channel in which no hybridization of a probe molecule to the amplicon is observed;
j) removing the first group of probe molecules from the cells;
k) repeating steps (g)-(j) for a number of iterations to exhaust the number of unique amplicon hybridization portions in the plurality of single-stranded DNA oligonucleotide probe molecules is exhausted, preferably 5-50 iterations, more preferably 8-12 iterations, more preferably 8 iterations,
thereby generating a binary code for each amplicon location; and
l) decoding the binary code for each amplicon location by mapping the binary code to a codebook which assigns the combined readout set of each barcoding oligonucleotide pair in the library to identification of a targeted molecule of the barcoding oligonucleotide pair, thereby identifying the polynucleotide molecule at the amplicon location, and
thereby detecting a polynucleotide molecule from a library of targeted molecules.

18. A composition comprising a barcoding oligonucleotide set, the set comprising:
a) a single-stranded DNA left primer oligonucleotide molecule comprising
i) a left universal bridge oligonucleotide hybridization portion;
ii) a first left readout portion (rs1);
iii) a second left readout portion (rs2);
iv) a left padlock bottom oligonucleotide hybridization portion; and
v) a left barcode portion (H1) comprising a nucleotide sequence complementary to a first targeted nucleotide sequence (H1′) of a targeted polynucleotide molecule; and
b) a single-stranded DNA padlock bottom oligonucleotide molecule comprising
i) a right primer oligonucleotide hybridization portion;
ii) a padlock bottom barcode portion (H2) comprising a nucleotide sequence complementary to a second targeted nucleotide sequence (H2′) of the targeted polynucleotide molecule; and
iii) a left primer oligonucleotide hybridization portion comprising a nucleotide sequence complementary to the left padlock bottom oligonucleotide hybridization portion of the left primer oligonucleotide; and
c) a single-stranded DNA right primer oligonucleotide molecule comprising
i) a right barcode portion (H3) comprising a nucleotide sequence complementary to a third targeted nucleotide sequence (H3′) of the targeted polynucleotide molecule nucleotide sequence;
ii) a right padlock bottom oligonucleotide hybridization portion comprising a nucleotide sequence complementary to the right primer oligonucleotide hybridization portion of the bottom padlock molecule;
iii) a first right primer readout portion (rs4);
iv) a second right primer readout portion (rs3); and
v) a right universal bridge oligonucleotide hybridization portion; and
d) a universal bridge oligonucleotide molecule comprising
i) a left primer oligonucleotide hybridization portion comprising a nucleotide sequence complementary to the left universal bridge oligonucleotide hybridization portion nucleotide sequence;
ii) an intervening bridge portion; and
iii) a right primer oligonucleotide hybridization portion comprising a nucleotide sequence complementary to the right universal bridge oligonucleotide hybridization portion nucleotide sequence
preferably wherein (d)(i) and (d)(iii) differ in melting temperature,
preferably such that hybridization of (d)(i) to (a)(i) may occur separately from the hybridization of (d)(iii) to (c)(v) under a first hybridization condition; and
may occur at the same as hybridization of (d)(iii) to (c)(v) under a second hybridization condition.

19. The composition of claim 18, further comprising, a single-stranded DNA oligonucleotide splint molecule comprising a nucleotide sequence complementary to the first left readout portion (rs1) nucleotide sequence, a single-stranded DNA oligonucleotide splint molecule comprising a nucleotide sequence complementary to the second left readout portion (rs2) nucleotide sequence, a single-stranded DNA oligonucleotide splint molecule comprising a nucleotide sequence complementary to the first right primer readout portion (rs4) nucleotide sequence, and/or a single-stranded DNA oligonucleotide splint molecule comprising a nucleotide sequence complementary to the second right primer readout portion (rs3) nucleotide sequence.

20. The composition of claim 18, further comprising an intermediate padlock bottom-binding oligonucleotide molecule comprising
i) a first intermediate primer readout portion (rs6);
ii) a first intermediate primer readout portion (rs5); and
iii) an amplicon padlock bottom barcode portion (aH2) comprising a nucleotide sequence complementary to the second targeted nucleotide sequence (H2′).
